(19) United States
(12) Patent Application Publication
(10) Pub. No.: US 2007/0264648 A1
(43) Pub. Date: Nov. 15, 2007

(54) Dna Oligomer, Genetic Marker and Dna Oligomer Set for Prediction of Onset of Side-Effect from Radiation Therapy, and Method for Predicting Onset of Side-Effect

(75) Inventors: Takashi Imai, Chiba (JP); Yoshinobu Harada, Chiba (JP); Mayumi Iwakawa, Chiba (JP)

Correspondence Address: SQUIRE, SANDERS & DEMPSEY L.L.P., 14TH FLOOR, 8000 TOWERS CRESCENT, TYSONS CORNER, VA, 22182, US

(73) Assignee: National Institute of Radiological Sciences

(21) Appl. No.: 11/660,658

(22) PCT Filed: Sep. 22, 2005
(86) PCT No.: PCT/JP05/17460

§ 371 Date: Mar. 27, 2007

(51) Int. Cl. C12Q 1/68; C07H 21/00

(52) U.S. Cl. 435/6; 536/23.1

(57) ABSTRACT

There are provided a DNA oligomer, a genetic marker, and a DNA oligomer set (PCR primer set) and a DNA oligomer (extension primer) for predicting a possibility of onset of a side-effect from radiation therapy for cancer by determining whether a specific base in a DNA sequence is a risk allele or a non-risk allele, and a method for predicting onset of a side-effect from radiation therapy. The DNA oligomer for a prediction of onset of a side-effect from radiation therapy has a DNA sequence of at least 10-241 contiguous bases with a 121st base from a sequence of any one of SEQ ID NOs: 1-173 in the Sequence Listing.

### TECHNICAL FIELD

The present invention relates to a DNA oligomer for a prediction of onset of a side-effect from radiation therapy using a single nucleotide polymorphism of gene as a criterion of determination; a genetic marker for a prediction of onset of a side-effect using the DNA oligomer for a prediction of onset of a side-effect from radiation therapy; a DNA oligomer set (PCR primer) and a DNA oligomer (extension primer) for obtaining the DNA oligomer for a prediction of onset of a side-effect from radiation therapy by determining SNP; and a method for predicting onset of a side-effect using the DNA oligomer for a prediction of onset of a side-effect from radiation therapy.

### BACKGROUND ART

Radiation therapy is one of effective local therapies against cancer, which is excellent from a viewpoint of maintaining body function and shape, since treatment is given without removing lesion, unlike surgery. In other words, unlike surgery in which body is cut with a scalpel, radiation therapy alleviates mental burden of patients and facilitates social integration of patients after operation, enhancing QOL (quality of life) of patients. Thus, radiation therapy is expected to develop more in the future.

Radiation therapy also has advantages in that it can be applied to a patient with complication or an aged person since radiation therapy alleviates physical burden. Further, a technique of stereotactic radiation therapy has been developed recently in which a position, a shape and a size of a lesion is accurately determined on the three-dimensional coordinate based on image information by CT or MRI, and radiation dosage can be made concentrated on the lesion.

As described above, radiation therapy is a very useful treatment for cancer. However, radiation may cause ulcer of skin or severe side-effect, such as intestinal perforation and pneumonia. In addition, some patients have high radiosensitivity which may necessitate cessation of radiation therapy.

It is considered that a presence of various levels of radiosensitivity is related to difference in DNA sequence of cancer patients. Such a difference in DNA sequence is generally called polymorphism, and classified into the following categories: (1) polymorphism in which one base—several tens of bases are deleted or inserted (insertion/deletion polymorphism), (2) polymorphism in which two bases—several tens of bases as one unit is repeated (VNTR or microsatellite polymorphism), and (3) polymorphism in which one base is replaced by other base (single nucleotide polymorphism).

With respect to the category (3), it is presumed that a single nucleotide polymorphism (SNP) is present per several hundreds—one thousand bases, thus 3-10 millions of SNP are considered to be present in a whole genome of human. It is further assumed that this SNP has a strong effect on “character” of individuals, such as features, personality and response to drugs.

### REFERENCE

Kenichi Matsubara and Yoshiyuki Sakaki (supervise eds.), Yusuke Nakamura (ed.), “GENOMICS OF POST-SEQUENCE (1) Strategy of SNP gene polymorphism”, Nakayama-Shoten Co. Ltd., pp. 2-3, June 2000

It is also considered that sensitivity to radiation is strongly affected by difference in DNA sequence of genes, including SNP. This in turn means that, if radiosensitivity of a cancer patient is known in advance by examining DNA sequence prior to radiation therapy, tailor-made radiation therapy can be realized.

However, almost no studies have been made regarding genes associated with radiosensitivity and SNP affecting on radio-sensitivity. Therefore, it was impossible to set a criterion for realizing tailor-made radiation therapy.

Accordingly, in order to realize tailor-made radiation therapy, it is desired to provide a DNA oligomer, a genetic marker, a DNA oligomer set (PCR primer set) and a DNA oligomer (extension primer) for a prediction of radiosensitivity level of a cancer patient, thus for a prediction of onset of a side-effect from radiation therapy, and a method for predicting onset of a side-effect from radiation therapy.

### DISCLOSURE OF THE INVENTION

The present inventors made intensive and extensive studies with a view toward solving the above-mentioned problems and realizing tailor-made radiation therapy, based on an idea of introducing SNP typing, with mainly focusing on cSNP (coding SNP), rSNP (regulatory SNP) and iSNP (intron SNP). As a result of determining DNA sequences of genes of patients with a side-effect after radiation therapy (hereinafter, simply referred to as “morbidity group”) and patients without a side-effect or with a minor side-effect (hereinafter, simply referred to as “non-morbidity group”), the inventors found a statistically-significant difference in appearance rate of allele between the morbidity group and the non-morbidity group, and completed the present invention.

In an aspect of the present invention, there are provided the following DNA oligomers for a prediction of onset of a side-effect from radiation therapy:

[1] A DNA oligomer for predicting a possibility of onset of a side-effect from radiation therapy for cancer by determining whether a specific base in a DNA sequence is a risk allele or a non-risk allele, the DNA oligomer having a DNA sequence of at least 10-241 contiguous bases with a 121st base from a sequence of any one of SEQ ID NOs: 1-173 in the Sequence Listing;

[2] A DNA oligomer for predicting a possibility of onset of a side-effect from radiation therapy for breast cancer by determining whether a specific base in a DNA sequence is a risk allele or a non-risk allele, the DNA oligomer having a DNA sequence of at least 10-241 contiguous bases with a 121st base from a sequence of SEQ ID NO: 1, 4, 7, 13, 15, 17, 19, 20, 26, 27, 30, 32, 44, 45, 48, 49, 50, 51, 53, 54, 58, 59, 60, 61, 62, 63, 65, 67, 73, 74, 77, 78, 82, 85, 88, 90, 91, 94, 97, 98, 106, 108, 112, 113, 116, 117, 126, 127, 132, 133, 136, 137, 138, 140, 143, 145, 147, 148, 151, 157, 159, 160, 162, 163, 165, 167, 170, 172 or 173 in the Sequence Listing;

[3] A DNA oligomer for predicting a possibility of onset of a side-effect from radiation therapy for cervical cancer by determining whether a specific base in a DNA sequence is a risk allele or a non-risk allele, the DNA oligomer having a DNA sequence of at least 10-241 contiguous bases with a 121st base from a sequence of SEQ ID NO: 2, 6, 22, 23, 29, 31, 34, 36, 37, 39, 41, 42, 43, 44, 46, 52, 56, 60, 64, 65, 68, 70, 71, 72, 75, 76, 80, 83, 86, 89, 91, 93, 98, 105, 110, 114, 118, 119, 121, 129, 134, 139, 141, 142, 144, 146, 149, 150, 152, 153, 154, 155, 157, 161 or 171 in the Sequence Listing;

[4] A DNA oligomer for predicting a possibility of onset of a side-effect from radiation therapy for prostate cancer by determining whether a specific base in a DNA sequence is a risk allele or a non-risk allele, the DNA oligomer having a DNA sequence of at least 10-241 contiguous bases with a 121st base from a sequence of SEQ ID NO: 3, 5, 8, 9, 10, 11, 12, 14, 16, 18, 19, 21, 24, 25, 28, 33, 35, 38, 39, 40, 45, 47, 48, 55, 57, 66, 69, 73, 79, 81, 84, 87, 92, 95, 96, 99, 100, 101, 102, 103, 104, 107, 109, 111, 115, 116, 120, 122, 123, 124, 125, 126, 128, 130, 131, 135, 151, 156, 158, 160, 164, 166, 168 or 169 in the Sequence Listing;

[5] A DNA oligomer for predicting a possibility of onset of a side-effect from radiation therapy for cancer during a period from a beginning of the therapy to an early stage, by determining whether a specific base in a DNA sequence is a risk allele or a non-risk allele, the DNA oligomer having a DNA sequence of at least 10-241 contiguous bases with a 121st base from a sequence of SEQ ID NO: 2, 5, 7, 8, 13, 15, 17, 19, 20, 22, 23, 24, 25, 26, 27, 28, 29, 31, 32, 33, 34, 36, 43, 44, 45, 48, 52, 56, 59, 60, 61, 64, 65, 66, 70, 71, 72, 73, 75, 76, 78, 80, 81, 86, 89, 90, 91, 92, 93, 94, 96, 98, 99, 102, 103, 105, 106, 112, 113, 114, 117, 118, 120, 121, 126, 127, 129, 132, 134, 137, 138, 140, 141, 143, 144, 146, 147, 149, 150, 151, 152, 153, 154, 157, 158, 160, 162, 163, 165, 167, 168, 169 or 171 in the Sequence Listing;

[6] A DNA oligomer for predicting a possibility of onset of a side-effect from radiation therapy for cancer during a late stage of 3 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 5, 8, 10, 11, 12, 18, 21, 33, 45, 48, 49, 53, 55, 58, 69, 77, 87, 100, 101, 104, 108, 109, 111, 115, 116, 122, 123, 124, 125, 126, 128, 133, 136, 145, 148, 151, 156, 159, 160, 162 or 170 in the Sequence Listing;

[7] A DNA oligomer for predicting a possibility of onset of a side-effect from radiation therapy for cancer during a late stage of 6 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 1, 3, 4, 5, 6, 9, 10, 11, 12, 14, 16, 19, 30, 35, 37, 38, 39, 40, 41, 42, 46, 47, 50, 51, 54, 57, 60, 62, 63, 67, 68, 73, 74, 79, 82, 83, 84, 85, 88, 95, 96, 97, 102, 107, 110, 119, 130, 131, 135, 139, 142, 155, 161, 164, 166, 172 or 173 in the Sequence Listing;

[8] A DNA oligomer for predicting a possibility of onset of a side-effect from radiation therapy for breast cancer during a period from a beginning of the therapy to an early stage, by determining whether a specific base in a DNA sequence is a risk allele or a non-risk allele, the DNA oligomer having a DNA sequence of at least 10-241 contiguous bases with a 121st base from a sequence of SEQ ID NO: 7, 13, 15, 17, 19, 20, 26, 27, 32, 44, 45, 59, 61, 65, 73, 78, 90, 91, 94, 98, 106, 112, 113, 117, 127, 132, 137, 138, 140, 143, 147, 157, 160, 162, 163, 165 or 167 in the Sequence Listing;

[9] A DNA oligomer for predicting a possibility of onset of a side-effect from radiation therapy for breast cancer during a late stage of 3 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 48, 49, 53, 58, 77, 108, 116, 126, 133, 136, 145, 148, 151, 159, 162 or 170 in the Sequence Listing;

[10] A DNA oligomer for predicting a possibility of onset of a side-effect from radiation therapy for breast cancer during a late stage of 6 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 1, 4, 30, 50, 51, 54, 60, 62, 63, 67, 74, 82, 85, 88, 97, 172 or 173 in the Sequence Listing;

[11] A DNA oligomer for predicting a possibility of onset of a side-effect from radiation therapy for cervical cancer during a period from a beginning of the therapy to an early stage, by determining whether a specific base in a DNA sequence is a risk allele or a non-risk allele, the DNA oligomer having a DNA sequence of at least 10-241 contiguous bases with a 121st base from a sequence of SEQ ID NO: 2, 22, 23, 29, 31, 34, 36, 43, 44, 52, 56, 60, 64, 65, 70, 71, 72, 75, 76, 80, 86, 89, 91, 93, 98, 105, 114, 118, 121, 129, 134, 141, 144, 146, 149, 150, 152, 153, 154, 157 or 171 in the Sequence Listing;

[12] A DNA oligomer for predicting a possibility of onset of a side-effect from radiation therapy for cervical cancer during a late stage of 6 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 6, 37, 39, 41, 42, 46, 68, 83, 110, 119, 139, 142, 155 or 161 in the Sequence Listing;

[13] A DNA oligomer for predicting a possibility of onset of a side-effect from radiation therapy for prostate cancer during a period from a beginning of the therapy to an early stage, by determining whether a specific base in a DNA sequence is a risk allele or a non-risk allele, the DNA oligomer having a DNA sequence of at least 10-241 contiguous bases with a 121st base from a sequence of SEQ ID NO: 5, 8, 24, 25, 28, 33, 48, 66, 81, 92, 96, 99, 102, 103, 120, 126, 151, 158, 168 or 169 in the Sequence Listing;

[14] A DNA oligomer for predicting a possibility of onset of a side-effect from radiation therapy for prostate cancer during a late stage of 3 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 5, 8, 10, 11, 12, 18, 21, 33, 45, 48, 55, 69, 87, 100, 101, 104, 109, 111, 115, 116, 122, 123, 124, 125, 128, 156 or 160 in the Sequence Listing;

(15) A DNA oligomer for predicting a possibility of onset of a side-effect from radiation therapy for prostate cancer during a late stage of 6 months from a beginning of the therapy, by determining whether a specific base in a DNA sequence is a risk allele or a non-risk allele, the DNA oligomer having a DNA sequence of at least 10-241 contiguous bases with a 121st base from a sequence of SEQ ID NO: 3, 5, 9, 10, 11, 12, 14, 16, 19, 35, 38, 39, 40, 47, 57, 73, 79, 84, 95, 96, 102, 107, 130, 131, 135, 164 or 166 in the Sequence Listing; and

[16] The DNA oligomer for a prediction of onset of a side-effect from radiation therapy according to any one of [1] to [15], wherein the DNA oligomer includes deletion, substitution or insertion of one to several bases except for the 121st base, or the DNA oligomer has a complementary DNA sequence thereto.

In another aspect of the present invention, there are provided:

[17] A genetic marker for a prediction of onset of a side-effect from radiation therapy, which is the DNA oligomer for a prediction of onset of a side-effect according to any one of [1] to [16], or a DNA oligomer that hybridizes with the DNA oligomer for a prediction of onset of a side-effect under stringent conditions;

[18] A DNA oligomer set consisting of a pair of DNA oligomers sequentially selected from DNA oligomers of SEQ ID NOs: 174-519 in the Sequence Listing, the SEQ ID NOs of the pair starting from even number;

[19] The DNA oligomer set according to [11], wherein the DNA oligomers of SEQ ID NOs: 174-519 in the Sequence Listing include deletion, substitution or insertion of one to several bases; and

[20] A DNA oligomer having a DNA sequence of SEQ ID NOs: 520-692 in the Sequence Listing, optionally including deletion, substitution or insertion of one to several bases.

In still another aspect of the present invention, there is provided:

[21] A method for predicting onset of a side-effect from radiation therapy in which determination is made using a DNA oligomer of any one of SEQ ID NOs: 1-173 in the Sequence Listing, comprising the following processes (a)-(g):
(a) a DNA sample is prepared from a specimen obtained from a cancer patient on whom radiation therapy is to be performed;
(b) DNA is amplified from the DNA sample prepared in the process (a) to obtain a DNA product;
(c) elongation reaction is performed using the DNA product amplified in the process (b) as a template, to obtain a DNA oligomer as elongation product;
(d) a DNA sequence of the DNA oligomer obtained in the process (c) is determined;
(e) a comparison is made between a base corresponding to a base at a 121st position of the DNA sequence of the DNA oligomer sequenced in the process (d) and a 121st base of the DNA sequence of any one of SEQ ID NOs: 1-173 in the Sequence Listing;
(f) it is determined whether the allele having the base compared in the process (e) is a risk allele or a non-risk allele; and
(g) a risk rate of onset of a side-effect from radiation in the cancer patient on whom the radiation therapy is to be performed is predicted, based on the result determined in the process (f).

In still another aspect of the present invention, there is provided [22] the method for predicting onset of a side-effect from radiation therapy according to [21], wherein the DNA sequence of any one of SEQ ID NOs: 1-173 in the Sequence Listing to be used for the comparison in the process (e) is: a DNA sequence of the DNA oligomer of SEQ ID NO: 1, 4, 7, 13, 15, 17, 19, 20, 26, 27, 30, 32, 44, 45, 48, 49, 50, 51, 53, 54, 58, 59, 60, 61, 62, 63, 65, 67, 73, 74, 77, 78, 82, 85, 88, 90, 91, 94, 97, 98, 106, 108, 112, 113, 116, 117, 126, 127, 132, 133, 136, 137, 138, 140, 143, 145, 147, 148, 151, 157, 159, 160, 162, 163, 165, 167, 170, 172 or 173 in the Sequence Listing, for a prediction of onset of a side-effect from radiation therapy for breast cancer; a DNA sequence of the DNA oligomer of SEQ ID NO: 2, 6, 22, 23, 29, 31, 34, 36, 37, 39, 41, 42, 43, 44, 46, 52, 56, 60, 64, 65, 68, 70, 71, 72, 75, 76, 80, 83, 86, 89, 91, 93, 98, 105, 110, 114, 118, 119, 121, 129, 134, 139, 141, 142, 144, 146, 149, 150, 152, 153, 154, 155, 157, 161 or 171 in the Sequence Listing, for a prediction of onset of a side-effect from radiation therapy for cervical cancer; a DNA sequence of the DNA oligomer of SEQ ID NO: 3, 5, 8, 9, 10, 11, 12, 14, 16, 18, 19, 21, 24, 25, 28, 33, 35, 38, 39, 40, 45, 47, 48, 55, 57, 66, 69, 73, 79, 81, 84, 87, 92, 95, 96, 99, 100, 101, 102, 103, 104, 107, 109, 111, 115, 116, 120, 122, 123, 124, 125, 126, 128, 130, 131, 135, 151, 156, 158, 160, 164, 166, 168 or 169 in the Sequence Listing, for a prediction of onset of a side-effect from radiation therapy for prostate cancer; a DNA sequence of the DNA oligomer of SEQ ID NO: 2, 5, 7, 8, 13, 15, 17, 19, 20, 22, 23, 24, 25, 26, 27, 28, 29, 31, 32, 33, 34, 36, 43, 44, 45, 48, 52, 56, 59, 60, 61, 64, 65, 66, 70, 71, 72, 73, 75, 76, 78, 80, 81, 86, 89, 90, 91, 92, 93, 94, 96, 98, 99, 102, 103, 105, 106, 112, 113, 114, 117, 118, 120, 121, 126, 127, 129, 132, 134, 137, 138, 140, 141, 143, 144, 146, 147, 149, 150, 151, 152, 153, 154, 157, 158, 160, 162, 163, 165, 167, 168, 169 or 171 in the Sequence Listing, for a prediction of onset of a side-effect from radiation therapy for cancer during a period from a beginning of the therapy to an early stage; a DNA sequence of the DNA oligomer of SEQ ID NO: 5, 8, 10, 11, 12, 18, 21, 33, 45, 48, 49, 53, 55, 58, 69, 77, 87, 100, 101, 104, 108, 109, 111, 115, 116, 122, 123, 124, 125, 126, 128, 133, 136, 145, 148, 151, 156, 159, 160, 162or170in the Sequence Listing, for a prediction of onset of a side-effect from radiation therapy for cancer during a late stage of 3 months from a beginning of the therapy; a DNA sequence of the DNA oligomer of SEQ ID NO: 1, 3, 4, 5, 6, 9, 10, 11, 12, 14, 16, 19, 30, 35, 37, 38, 39, 40, 41, 42, 46, 47, 50, 51, 54, 57, 60, 62, 63, 67, 68, 73, 74, 79, 82, 83, 84, 85, 88, 95, 96, 97, 102, 107, 110, 119, 130, 131, 135, 139, 142, 155, 161, 164, 166, 172 or 173in the Sequence Listing, for a prediction of onset of a side-effect from radiation therapy for cancer during a late stage of 6 months from a beginning of the therapy; a DNA sequence of the DNA oligomer of SEQ ID NO: 7, 13, 15, 17, 19, 20, 26, 27, 32, 44, 45, 59, 61, 65, 73, 78, 90, 91, 94, 98, 106, 112, 113, 117, 127, 132, 137, 138, 140, 143, 147, 157, 160, 162, 163, 165 or 167 in the Sequence Listing, for a prediction of onset of a side-effect from radiation therapy for breast cancer during a period from a beginning of the therapy to an early stage; a DNA sequence of the DNA oligomer of SEQ ID NO: 48, 49, 53, 58, 77, 108, 116, 126, 133, 136, 145, 148, 151, 159, 162 or 170 in the Sequence Listing, for a prediction of onset of a side-effect from radiation therapy for breast cancer during a late stage of 3 months from a beginning of the therapy; a DNA sequence of the DNA oligomer of SEQ ID NO: 1, 4, 30, 50, 51, 54, 60, 62, 63, 67, 74, 82, 85, 88, 97, 172 or 173 in the Sequence Listing, for a prediction of onset of a side-effect from radiation therapy for breast cancer during a late stage of 6 months from a beginning of the therapy; a DNA sequence of the DNA oligomer of SEQ ID NO: 2, 22, 23, 29, 31, 34, 36, 43, 44, 52, 56, 60, 64, 65, 70, 71, 72, 75, 76, 80, 86, 89, 91, 93, 98, 105, 114, 118, 121, 129, 134, 141, 144, 146, 149, 150, 152, 153, 154, 157 or 171 in the Sequence Listing, for a prediction of onset of a side-effect from radiation therapy for cervical cancer during a period from a beginning of the therapy to an early stage; a DNA sequence of the DNA oligomer of SEQ ID NO: 6, 37, 39, 41, 42, 46, 68, 83, 110, 119, 139, 142, 155 or 161 in the Sequence Listing, for a prediction of onset of a side-effect from radiation therapy for cervical cancer during a late stage of 6 months from a beginning of the therapy; a DNA sequence of the DNA oligomer of SEQ ID NO: 5, 8, 24, 25, 28, 33, 48, 66, 81, 92, 96, 99, 102, 103, 120, 126, 151, 158, 168 or 169 in the Sequence Listing, for a prediction of onset of a side-effect from radiation therapy for prostate cancer during a period from a beginning of the therapy to an early stage; a DNA sequence of the DNA oligomer of SEQ ID NO: 5, 8, 10, 11, 12, 18, 21, 33, 45, 48, 55, 69, 87, 100, 101, 104, 109, 111, 115, 116, 122, 123, 124, 125, 128, 156 or 160 in the Sequence Listing, for a prediction of onset of a side-effect from radiation therapy for prostate cancer during a late stage of 3 months from a beginning of the therapy; and a DNA sequence of the DNA oligomer of SEQ ID NO: 3, 5, 9, 10, 11, 12, 14, 16, 19, 35, 38, 39, 40, 47, 57, 73, 79, 84, 95, 96, 102, 107, 130, 131, 135, 164 or 166 in the Sequence Listing, for a prediction of onset of a side-effect from radiation therapy for prostate cancer during a late stage of 6 months from a beginning of the therapy.

By using the DNA oligomer and the genetic marker for a prediction of onset of a side-effect from radiation therapy of the present invention, a specific single nucleotide polymorphism (SNP) in a DNA sequence of gene can be detected, thereby predicting a risk rate of onset of a side-effect from radiation therapy. Therefore, the present invention supports realization of tailor-made radiation therapy.

By using the DNA oligomer set (PCR primer) of the present invention, DNA containing a specific single nucleotide polymorphism (SNP) can be easily amplified from a DNA sample prepared from a subject, such as cancer patient. Further, by using the DNA oligomer (extension primer) of the present invention, a base type of the specific SNP site can be easily determined. Therefore, a method for predicting a risk rate of onset of a side-effect from radiation therapy is simplified.

According to the method for predicting onset of a side-effect, based on information of SNP contained in the DNA sample obtained from a cancer patient on whom radiation therapy is to be performed, a risk rate of onset of a side-effect from radiation can be predicted. In other words, tailor-made radiation therapy can be preformed.

According to the present invention, for breast cancer, cervical cancer, and prostate cancer, a risk rate of onset of a side-effect from radiation can be predicted in advance, at various stages including an early stage (less than 3 month from the beginning of the therapy), a late stage of 3 months and a late stage of 6 months from the beginning of the therapy.

The various aspects and effects and further effects and features will become more apparent by describing in detail following illustrative, non-limiting embodiments, with reference to the accompanying drawings.

### BRIEF DESCRIPTION OF DRAWINGS

FIG. 1 is a flow chart explaining a method for predicting onset of a side-effect from radiation therapy of the present invention.

### BEST MODE FOR CARRYING OUT THE INVENTION

Embodiments of the present invention will be described in detail below. However, the present invention should not be construed as being limited by the following descriptions.

[1. DNA Oligomer (1) and Genetic Marker]

An expression “DNA oligomer for a prediction of onset of a side-effect from radiation therapy” in the present invention means an oligonucleotide (DNA oligomer) having at least 10-241 bases from DNA sequence of SEQ ID NOs: 1-173 in the Sequence Listing, and including a 121st base of DNA sequence of SEQ ID NOs: 1-173 in the Sequence Listing.

In the present invention, there is no limitation with respect to a length of the DNA sequence, as long as the DNA sequence has 10-241 contiguous base sequence from the above-mentioned sequences in the Sequence Listing, and at the same time includes the 121st base from the above-mentioned sequences in the Sequence Listing. In other words, the DNA oligomer may have a length of 10-241 bases as described above, or a length of more than 241 bases. For example, when the DNA sequence is present on chromosome, a further longer DNA oligomer (such as DNA oligomer containing the 121st base, having a length of contiguous 250 bases, 500 bases or more) is possible, which falls in the DNA oligomer of NOs: 1-173 in the Sequence Listing of the present invention.

In the present invention, a risk allele is located at the 121st base. The expression “risk allele” means an allele at a specific SNP site that a person who is likely to have onset of a side-effect (disorder) from radiation therapy has, and bases at the allele are different between a person who is likely to have onset of a side-effect and a person who is not likely to have onset of a side-effect. Therefore, if the risk allele is determined by the SNP typing, a risk rate of onset of a side-effect from radiation therapy can be predicted.

It should be noted that there is no limitation with respect to the location of the “121st” base in a DNA oligomer, as long as the 121st base designated in the present invention is identified. In other words, the SNP site as a risk allele may be located somewhere in the middle of a DNA sequence, or at 5′ end or 3′ end.

On the other hand, in gene or chromosomal DNA containing a DNA sequence which is identical or substantially identical to the DNA sequence of SEQ ID NOs: 1-173 in the Sequence Listing of the present invention, an allele which is located at an SNP site equivalent to the above-mentioned risk allele but has a different base combination from that of the risk allele is called “non-risk allele” in the present invention.

DNA oligomers of SEQ ID NOs: 1-173 in the Sequence Listing (or information of DNA sequences thereof) can be used for predicting a possibility of onset of a side-effect from radiation therapy for cancer, by determining whether a specific base in the DNA sequence is a risk allele or a non-risk allele. Especially, the DNA oligomer can be suitably used in configurations described in the following (1)-(14).

(1) For a prediction of onset of a side-effect from radiation therapy for breast cancer, DNA oligomers having DNA sequences of SEQ ID NOs: 1, 4, 7, 13, 15, 17, 19, 20, 26, 27, 30, 32, 44, 45, 48, 49, 50, 51, 53, 54, 58, 59, 60, 61, 62, 63, 65, 67, 73, 74, 77, 78, 82, 85, 88, 90, 91, 94, 97, 98, 106, 108, 112, 113, 116, 117, 126, 127, 132, 133, 136, 137, 138, 140, 143, 145, 147, 148, 151, 157, 159, 160, 162, 163, 165, 167, 170, 172 and/or 173 in the Sequence Listing are suitably used.

(2) For a prediction of onset of a side-effect from radiation therapy for cervical cancer, DNA oligomers having DNA sequence of SEQ ID NOs: 2, 6, 22, 23, 29, 31, 34, 36, 37, 39, 41, 42, 43, 44, 46, 52, 56, 60, 64, 65, 68, 70, 71, 72, 75, 76, 80, 83, 86, 89, 91, 93, 98, 105, 110, 114, 118, 119, 121, 129, 134, 139, 141, 142, 144, 146, 149, 150, 152, 153, 154, 155, 157, 161 and/or 171 in the Sequence Listing are suitably used.

(3) For a prediction of onset of a side-effect from radiation therapy for prostate cancer, DNA oligomers having DNA sequence of SEQ ID NOs: 3, 5, 8, 9, 10, 11, 12, 14, 16, 18, 19, 21, 24, 25, 28, 33, 35, 38, 39, 40, 45, 47, 48, 55, 57, 66, 69, 73, 79, 81, 84, 87, 92, 95, 96, 99, 100, 101, 102, 103, 104, 107, 109, 111, 115, 116, 120, 122, 123, 124, 125, 126, 128, 130, 131, 135, 151, 156, 158, 160, 164, 166, 168 and/or 169 in the Sequence Listing are suitably used.

(4) For a prediction of onset of a side-effect from radiation therapy for cancer during a period from a beginning of the therapy to an early stage, DNA oligomers having DNA sequence of SEQ ID NOs: 2, 5, 7, 8, 13, 15, 17, 19, 20, 22, 23, 24, 25, 26, 27, 28, 29, 31, 32, 33, 34, 36, 43, 44, 45, 48, 52, 56, 59, 60, 61, 64, 65, 66, 70, 71, 72, 73, 75, 76, 78, 80, 81, 86, 89, 90, 91, 92, 93, 94, 96, 98, 99, 102, 103, 105, 106, 112, 113, 114, 117, 118, 120, 121, 126, 127, 129, 132, 134, 137, 138, 140, 141, 143, 144, 146, 147, 149, 150, 151, 152, 153, 154, 157, 158, 160, 162, 163, 165, 167, 168, 169 and/or 171 in the Sequence Listing are suitably used.

(5) For a prediction of onset of a side-effect from radiation therapy for cancer during a late stage of 3 months from a beginning of the therapy, DNA oligomers having DNA sequence of SEQ ID NOs: 5, 8, 10, 11, 12, 18, 21, 33, 45, 48, 49, 53, 55, 58, 69, 77, 87, 100, 101, 104, 108, 109, 111, 115, 116, 122, 123, 124, 125, 126, 128, 133, 136, 145, 148, 151, 156, 159, 160, 162 and/or 170 in the Sequence Listing are suitably used.

(6) For a prediction of onset of a side-effect from radiation therapy for cancer during a late stage of 6 months from a beginning of the therapy, DNA oligomers having DNA sequence of SEQ ID NOs: 1, 3, 4, 5, 6, 9, 10, 11, 12, 14, 16, 19, 30, 35, 37, 38, 39, 40, 41, 42, 46, 47, 50, 51, 54, 57, 60, 62, 63, 67, 68, 73, 74, 79, 82, 83, 84, 85, 88, 95, 96, 97, 102, 107, 110, 119, 130, 131, 135, 139, 142, 155, 161, 164, 166, 172 and/or 173 in the Sequence Listing are suitably used.

(7) For a prediction of onset of a side-effect from radiation therapy for breast cancer during a period from a beginning of the therapy to an early stage, DNA oligomers having DNA sequence of SEQ ID NOs: 7, 13, 15, 17, 19, 20, 26, 27, 32, 44, 45, 59, 61, 65, 73, 78, 90, 91, 94, 98, 106, 112, 113, 117, 127, 132, 137, 138, 140, 143, 147, 157, 160, 162, 163, 165 and/or 167 in the Sequence Listing are suitably used.

(8) For a prediction of onset of a side-effect from radiation therapy for breast cancer during a late stage of 3 months from a beginning of the therapy, DNA oligomers having DNA sequence of SEQ ID NOs: 48, 49, 53, 58, 77, 108, 116, 126, 133, 136, 145, 148, 151, 159, 162 and/or 170 in the Sequence Listing are suitably used.

(9) For a prediction of onset of a side-effect from radiation therapy for breast cancer during a late stage of 6 months from a beginning of the therapy, DNA oligomers having DNA sequence of SEQ ID NOs: 1, 4, 30, 50, 51, 54, 60, 62, 63, 67, 74, 82, 85, 88, 97, 172 and/or 173 in the Sequence Listing are suitably used.

(10) For a prediction of onset of a side-effect from radiation therapy for cervical cancer during a period from a beginning of the therapy to an early stage, DNA oligomers having DNA sequence of SEQ ID NOs: 2, 22, 23, 29, 31, 34, 36, 43, 44, 52, 56, 60, 64, 65, 70, 71, 72, 75, 76, 80, 86, 89, 91, 93, 98, 105, 114, 118, 121, 129, 134, 141, 144, 146, 149, 150, 152, 153, 154, 157 and/or 171 in the Sequence Listing are suitably used.

(11) For a prediction of onset of a side-effect from radiation therapy for cervical cancer during a late stage of 6 months from a beginning of the therapy, DNA oligomers having DNA sequence of SEQ ID NOs: 6, 37, 39, 41, 42, 46, 68, 83, 110, 119, 139, 142, 155 and/or 161 in the Sequence Listing are suitably used.

(12) For a prediction of onset of a side-effect from radiation therapy for prostate cancer during a period from a beginning of the therapy to an early stage, DNA oligomers having DNA sequence of SEQ ID NOs: 5, 8, 24, 25, 28, 33, 48, 66, 81, 92, 96, 99, 102, 103, 120, 126, 151, 158, 168 and/or 169 in the Sequence Listing are suitably used.

(13) For a prediction of onset of a side-effect from radiation therapy for prostate cancer during a late stage of 3 months from a beginning of the therapy, DNA oligomers having DNA sequence of SEQ ID NOs: 5, 8, 10, 11, 12, 18, 21, 33, 45, 48, 55, 69, 87, 100, 101, 104, 109, 111, 115, 116, 122, 123, 124, 125, 128, 156 and/or 160 in the Sequence Listing are suitably used.

(14) For a prediction of onset of a side-effect from radiation therapy for prostate cancer during a late stage of 6 months from a beginning of the therapy, DNA oligomers having DNA sequence of SEQ ID NOs: 3, 5, 9, 10, 11, 12, 14, 16, 19, 35, 38, 39, 40, 47, 57, 73, 79, 84, 95, 96, 102, 107, 130, 131, 135, 164 and/or 166 in the Sequence Listing are suitably used.

The length of the DNA oligomer containing a risk allele to be used in the present invention, as a DNA oligomer having 10-241 contiguous bases with the 121st base (risk allele), may be appropriately set.

For example, when the DNA oligomer is used as a genetic marker, such as labeled probe, the base length is preferably 20-200, but the length may be 30-150 or 35-100, or may even be 200 or more. Selection of an appropriate length facilitates, for example, specific hybridization or easy determination of difference in migration distance in electrophoresis, leading to an appropriate SNP typing. Too short base length is not preferred since non-specific hybridization may occur, while too long base length is not preferred since determination of a difference in migration distance becomes difficult.

According to the present invention, by analyzing/detecting the above-mentioned DNA oligomer, a presence of genetic factors affecting a likelihood of onset of a side-effect from radiation therapy is determined, and based on this result, a possibility of onset of a side-effect from radiation is predicted.

Therefore, by comparing the analyzed DNA sequence with a DNA sequence of any one of SEQ ID NOs: 1-173 in the Sequence Listing, it is confirmed whether the sequence matches the risk allele or the non-risk allele specified in the present invention, to thereby determine whether the analyzed DNA sequence is a base sequence with likelihood of onset of a side-effect from radiation (morbidity group) or a base sequence with unlikelihood of onset of a side-effect (non-morbidity group). In other words, it can be determined that a person with the risk allele has genetic factors that tend to contribute to onset of a side-effect from radiation therapy, as compared with a person with the non-risk allele.

The DNA oligomer according to the present invention may include deletion, substitution or insertion of one to several bases except for the 121st base, or may be a complementary strand thereto. In other words, even though a DNA oligomer includes substitution, deletion, insertion and the like of the base other than the above-specified SNP site, such a DNA oligomer falls in the DNA oligomer of the present invention, if the DNA oligomer has the SNP site specified by the claims and the Sequence Listing of the present invention and has a DNA sequence which is substantially the same as or complementary to the DNA sequence defined by the present invention.

In addition, for such a DNA oligomer, a DNA product may be preferably used which was replicated or amplified by various polymerases, such as DNA polymerase, especially heat-resistant polymerase (e.g., Taq polymerase).

Further, the DNA oligomer of the present invention may be preferably used for directly analyzing a DNA sequence of the DNA oligomer. For direct analysis of the DNA sequence of the DNA oligomer, a conventional DNA sequencer and mass spectrograph may be preferably used.

In addition, the DNA oligomer of the present invention is suitable to be used as a genetic marker, such as probe.

When the DNA oligomer of the present invention is used as genetic marker, it is more preferred that the genetic marker is labeled with fluorochrome, radioactive isotope or the like at an end of a DNA sequence or any of bases in the DNA sequence (labeled probe). A labeled genetic marker facilitates detection thereof by simply measuring fluorescence intensity, radiation dose and the like, or by simply exposing an X-ray film with fluorescence, or with radiation. For a measurement of radiation dose or fluorescence intensity, conventional radiation detectors or fluorophotometers are preferably used. In addition, by labeling with fluorochrome, the DNA sequence can be appropriately analyzed with a DNA sequencer.

Examples of the radioactive isotopes to be used for labeling include commonly used radioactive isotopes, such as ³²P and ³⁵S. Examples of the fluorochromes to be used for labeling include commonly used fluorochromes, such as FAM™, Yakima Yellow™, VIC™, TAMRA™, ROX™, Cy3™, Cy5™, HEX™, TET™ and FITC.

As described above, the DNA oligomer for a prediction of onset of a side-effect from radiation therapy of the present invention is suitably used for detection of single nucleotide polymorphism, i.e. SNP typing, by directly analyzing the DNA sequence or using the DNA oligomer as a genetic marker. It should be noted that, in general, the term “polymorphism” is defined as a change in base present at a ratio of 1% or more in a population. However, the term “polymorphism” used herein is not limited to this definition, and includes a change in base at a ratio of less than 1%.

[2. DNA Oligomer Set]It is preferred that the DNA oligomer of SEQ ID NOs: 174-519 in the Sequence Listing be used as DNA oligomer sets each consisting of a pair of DNA oligomers sequentially selected from SEQ ID NO: 174.

By selecting a DNA oligomer set appropriate for DNA amplification from the DNA oligomer sets, any of the DNA oligomer of SEQ ID NOs: 1-173 in the Sequence Listing can be specifically amplified. It should be noted that, of the DNA oligomer set, a DNA oligomer with a smaller sequence identification number is a forward primer, and a DNA oligomer with a larger sequence identification number is a reverse primer. A part flanked by sequences corresponding to these two primers is a subject to be amplified by PCR.

As described above, the DNA oligomer sets shown in SEQ ID NOs: 174-519 in the Sequence Listing are suitably used, especially for PCR (Polymerase Chain Reaction), and thus the DNA oligomer sets of the present invention are preferably used as PCR amplification primer set. Accordingly, with a use of the PCR amplification primer set, a DNA oligomer with a specific DNA sequence, i.e. DNA oligomer containing a specific SNP site (in the present invention, the 121st base is the specific SNP site) shown in SEQ ID NOs: 1-173 in the Sequence Listing can be securely, simply and specifically amplified.

It should be noted that each DNA oligomer contained in the DNA oligomer set may have a DNA sequence that includes deletion, substitution or insertion of one to several bases. Further, to each of the DNA oligomers of SEQ ID NOs: 174-519, an appropriate recognition sequence for restriction endonuclease (for example, a DNA oligomer of approximately 10 bases, such as 5′-ACGTTGGATG-3′ (SEQ ID NO: 693)) may be added at upstream of 5′ end of the DNA oligomer, if desired. Such an added sequence has an effect of stabilizing amplification reaction by PCR. It should be noted that the base sequence to be added is not limited to one mentioned above, and any sequence may be used as long as the sequence has a similar effect.

[3. DNA Oligomer (2)]

In addition, each DNA oligomer of SEQ ID NOs: 520-692 in the Sequence Listing is designed so that a 3′ end thereof is positioned adjacent to a base as a specific SNP site of a corresponding DNA oligomer of SEQ ID NOs: 1-173 in the Sequence Listing. Therefore, when the DNA oligomer is enzymatically elongated by one to several bases, the generated DNA oligomer has different length due to polymorphism. Further, this DNA oligomer also may optionally include deletion, substitution or insertion of one to several bases.

Each of the DNA oligomers of SEQ ID NOs: 520-692 in the Sequence Listing preferably has a base length of 10-24, more preferably 15-24, still more preferably 17-24, at the 3′ end thereof which is to be adjacent to the risk allele. The length may be appropriately altered depending on a GC amount in the DNA oligomer, hybridization conditions and the like.

For enzymatically elongating the DNA oligomer by one to several bases, there can be mentioned, for example, a method in which a DNA oligomer is elongated by only single base (1-base primer extension), by using a DNA polymerase as a DNA synthetase and dideoxynucleoside triphosphate (ddNTP) as an analogue of deoxynucleoside triphosphate (dNTP). Accordingly, the DNA oligomer is suitably used for SNP typing in which a base as a risk allele is analyzed.

In this manner, a difference in a length of the DNA product may be accurately determined with a mass spectrometer (MassEXTEND™ method, SEQUENOM, Inc.), by, for example, elongating one allele by one base, and the other allele by 3 bases. It is preferred that an elongation number of base is selected for each SNP site, based on efficiency in elongation according to the flanking sequences of the polymorphism site for each SNP site.

[4. SNP Typing]

Examples of techniques to be used for SNP typing include a technique using primer extension, a technique using hybridization, a technique using DNA cleavage, a technique using ligation and the like.

(4-1. SNP Typing Technique-1)

As for a technique using primer extension, there can be mentioned the above-mentioned 1-base primer extension (Syvanen, A. C. et al., Genomics, 8, 684-692 (1990)), MALDI-TOF/MS using the same (Ross, P., et al. Nat Biotechnol, 16, 1347-1351 (1998); Buetow, K. H. et al., Proc Natl Acad Sci USA, 98, 581-584 (2001); “Strategy of SNP gene polymorphism”, Kenichi Matsubara and Yoshiyuki Sakaki, Nakayama-Shoten, Co. Ltd., pp. 106-117), allele-specific primer extension (Uggozzoli, L. et al., Genet Anal Tech Appl, 9, 107-112 (1992)), an APEX method (in the arrayed primer extension) (Shumaker, J. M. et al., Hum Mutat, 7, 346-354 (1996)) and the like. Among these, MALDI-TOF/MS is especially preferred since SNP typing of a large amount of sample can be easily conducted at a time.

This MALDI-TOF/MS (matrix assisted laser desorption ionization time-of-flight/mass spectrometry) is a very efficient method, since a DNA sequence of a DNA product obtained from a DNA sample can be directly determined and compared.

The MALDI-TOF/MS is one of genotyping methods that process a large amount of sample at high speed, as described above. In this method, mass spectrograph conventionally used in biology/chemistry field is applied to SNP typing. Since the method uses mass spectrograph, a principle is that a difference by polymorphism is correlated with a difference in molecules in a certain form and a base sequence is determined by detecting a difference in molecular weight. Simply put, MALDI-TOF/MS utilizes mass spectrograph together with primer extension to determine a difference of base at the SNP site.

Specifically, first, a DNA sample was extracted from a cancer patient on whom radiation therapy is to be performed. In this case, DNA containing a base at a site corresponding to the SNP site shown in SEQ ID NOs: 1-173 in the Sequence Listing of the present invention is prepared by amplifying by, for example, PCR. Next, by using the PCR product as a template, ddNTP primer extension reaction is performed on a genotyping primer (a primer having a sequence complementary to a sequence on the 3′ side extending from a base next in the 3′ side direction to the base at a site corresponding to the SNP site shown in SEQ ID NOs: 1-173 in the Sequence Listing, or in a case of a complementary strand to SEQ ID NOs: 1-173 in the Sequence Listing, a primer having a sequence complementary to the complementary strand), to thereby elongate one to several bases. It is preferred that the PCR product used in this reaction be purified to remove PCR amplification primer, and that the genotyping primer [extension primer (shown in SEQ ID NOs: 520-692 in the Sequence Listing)] typically have 15 bp or more. In addition, in the primer extension reaction, excessive genotyping primer is generally added in an amount more than 10 times as much as the amount of PCR product, but the present invention is not limited to this amount. Thermal cycle conditions for PCR are appropriately selected, but conditions in which approximately 30-60% of the genotyping primer is elongated are preferred. For example, appropriate elongation efficiency can be attained by repeating a thermal cycle 25 times, which includes 2 different temperatures of 94° C. and 37° C. Next, a primer extension reaction product is spotted on a MALDI plate, mass is measured, and mass spectrogram is created. By analyzing the created mass spectrogram, DNA is sequenced, and thus, only one reaction is required for determining whether the base at a site corresponding to the SNP site shown in SEQ ID NOs: 1-173 in the Sequence Listing of the present invention is a base sequence with likelihood of onset of a side-effect from radiation (morbidity group) or a base sequence with unlikelihood of onset of a side-effect (non-morbidity group).

As described above, with MALDI-TOF/MS, a large amount of SNP typing can be realized at a high speed by directly determining DNA sequence using mass spectrograph. However, the present invention is not limited to this method, in order to determine DNA sequence. As for a method for determining a base sequence of DNA using a DNA sample, SNP typing with a DNA sequencer using slab gel or multicapillary, for example, can be mentioned.

(4-2. SNP Typing Technique-2)

As for a technique using hybridization, there can be mentioned, for example, TaqMan PCR (Livak, K. J. et al., PCR Methods Appl., 4, 357-362 (1995); “Strategy of SNP gene polymorphism”, Kenichi Matsubara and Yoshiyuki Sakaki, Nakayama-Shoten, Co., Ltd., pp. 94-105).

Specifically, first, a DNA sample was extracted from a cancer patient on whom radiation therapy is to be performed. A 5′ end of a DNA oligomer (probe) selected in advance for hybridization with a DNA containing a base at a site corresponding to the SNP site shown in SEQ ID NOs: 1-173 in the Sequence Listing of the present invention (i.e., any one of the DNA oligomers of SEQ ID NOs: 1-173 in the Sequence Listing or a complementary strand thereto) is labeled with reporter fluorescence. In the present invention, examples of the reporter fluorescent substance include, but are not limited to, the above-mentioned FAM and VIC. Further, a 3′ end of the probe is labeled with quencher substance. In the present invention, there is no limitation with respect to the quencher substance as long as it can quench reporter fluorescence. For example, examples include Dabcyl, BHQ1, BHQ2, Eclipse™ Dark Quencher and ElleQuencher™.

Next, the probe labeled with reporter fluorescence and quencher substance, which is for hybridization with DNA containing a base at a site corresponding to the SNP site shown in SEQ ID NOs: 1-173 in the Sequence Listing of the present invention, is hybridized with DNA prepared from the cancer patient on whom radiation therapy is to be performed. Subsequently, DNA containing a base at a site corresponding to the SNP site shown in SEQ ID NOs: 1-173 in the Sequence Listing of the present invention is amplified with DNA polymerase having 5′→3′ exonuclease activity. As a result, the reporter fluorescence-labeled part of the nucleotide probe labeled with reporter fluorescence and quencher substance is cleaved, and the reporter fluorescence is released. In the present invention, a preferable example of the DNA polymerase having 5′→3′ exonuclease activity includes, but is not limited to, Taq DNA polymerase. Next in this method, the released reporter fluorescence is detected, and emission of the reporter fluorescence is compared with a control. It should be noted that, in the present method, SNP typing can be made with one reaction, by introducing 2 different nucleotide probes labeled with different reporter fluorescences for each of a case where a base at a site corresponding to the SNP site shown in SEQ ID NOs: 1 -173 in the Sequence Listing of the present invention is a base sequence with likelihood of onset of a side-effect from radiation (morbidity group), and a case where the base at the site is a base sequence with unlikelihood of onset of a side-effect (non-morbidity group).

(4-3. SNP Typing Technique-3)

As for another technique using hybridization, there can be mentioned, for example, allele specific oligonucleotide (ASO) hybridization (Baner, J. et al., Nucleic Acids Res, 26, 5073-5078 (1998)).

Specifically, in order to detect only a variation of a specific position, a DNA oligomer (genetic marker) containing a base sequence which is believed to have a variation is prepared in advance, and the DNA oligomer is hybridized with DNA of the DNA sample. When a variation is present, formation efficiency of hybrid decreases, and thus an SNP is detected by Southern blotting or a technique in which quenching occurs when a special fluorescence reagent is intercalated into a gap of the hybrid. Further, by subjecting the detected base sequence to a DNA sequencer or the like, a base type that relates to SNP can be directly determined. Subsequently, by comparing the determined base types, SNP typing can be made with respect to whether the DNA has a base sequence with likelihood of onset of a side-effect from radiation (morbidity group) or a base sequence with unlikelihood of onset of a side-effect (non-morbidity group).

As for a technique using DNA cleavage, there can be mentioned, for example, an Invader method (Lyamichev, V. et al., Nat Biotechnol, 17,292-296 (1999); “Strategy of SNP gene polymorphism”, Kenichi Matsubara and Yoshiyuki Sakaki, Nakayama-Shoten, Co. Ltd., pp. 94-105).

Specifically, first, a DNA sample was extracted from a cancer patient on whom radiation therapy is to be performed. Next, an allele probe is synthesized that has: a base sequence complementary to base sequence on the 5′ side extending from a base at a site corresponding to the SNP site shown in SEQ ID NOs: 1-173 in the Sequence Listing of the present invention selected in advance; and a base sequence (flap) that does not hybridize with the selected DNA sequence on the 3′ side extending from a base next in the 3′ side direction to the base at the site corresponding to the SNP site, but is complementary to a part of a base sequence of an invader probe which will be described below.

In addition, an invader probe is synthesized which has: a base (arbitrary base) corresponding to the SNP site shown in SEQ ID NOs: 1-173 in the Sequence Listing of the present invention at 3′ end thereof; and a complementary sequence to a base sequence on the 3′ side extending from a base next in the 3′ side direction to the base at the site corresponding to the SNP site.

Next, these allele probe and invader probe are hybridized with a template DNA in the DNA sample. Upon hybridization, a base of the invader probe equivalent to a base at a site corresponding to the SNP site of DNA sequence of SEQ ID NOs: 1-173 in the Sequence Listing of the present invention (arbitrary base) invades a space between the template DNA and the allele probe. By Cleavase™ which is an enzyme having an endonuclease activity to recognize the invasion site and to cleave at a position between a base of the allele probe corresponding to the site and a base on the 3′ side next in the 3′ end direction to such a base of the allele probe, a flap part of the allele probe is cleaved and released. Next, the released flap is hybridized with an FRET (fluorescence resonance energy transfer) probe which has a complementary sequence with the above and is labeled with reporter fluorescence and quencher substance. A base sequence on the 5′ side of the FRET probe is capable of complementarily binding to the probe itself. A base sequence on the 3′ side of the FRET probe is complementary to the flap, as described above. In addition, the base sequence on the 5′ side which is capable of complementarily binding with the probe itself is labeled with reporter fluorescence on the 5′ end thereof, and quencher substance on the 3′ side of the 5′ end thereof. When the base at the 3′ end of the released flap is hybridized with the FRET probe, the probe invades a site of complementary binding where reporter fluorescence is labeled, and a structure recognizable by Cleavase™ is formed. Therefore, in the present method, by measuring reporter fluorescence that was released by cleavage of reporter fluorescence-labeled part by Cleavase™ and by comparing fluorescence intensity measured, SNP typing can be made with respect to whether the DNA has a base sequence with likelihood of onset of a side-effect from radiation (morbidity group) or a base sequence with unlikelihood of onset of a side-effect (non-morbidity group).

(4-4. SNP Typing Technique-4)

As for a technique using ligation, there can be mentioned, for example, RCA (rolling circle amplification) method (Lizardi, P. M et al., Nat Genet, 19, 225-232 (1998); Magnus J., TECHNOLOGY DEVELOPMENT FOR GENOME AND POLYMORPHISM ANALYSIS, 23-24 (2003), Karolinska University Press, Stockholm, Sweden; “Strategy of SNP gene polymorphism”, Kenichi Matsubara and Yoshiyuki Sakaki, Nakayama-Shoten, Co., Ltd., pp. 118-127).

In the RCA method, a long complementary DNA strand is synthesized by continuously synthesizing DNA while DNA polymerase exhibiting no 5′→3′ exonuclease activity under specific conditions repeatedly circles along a circular single-stranded DNA as a template.

Therefore in the RCA method, distinction of alleles is performed by determining a presence of DNA amplification. Specifically, a DNA oligomer to be used as a template for synthesis by DNA polymerase is provided as a linear chain, and a ligation reaction is implemented between a genomic DNA as a template for ligation reaction and an SNP site set at 3′ end and 5′ end of the linear chain DNA oligomer. When a ligation is completed and a circular single-stranded DNA is formed, RCA reaction proceeds, and a long complementary DNA strand can be obtained. On the other hand, when the DNA oligomer is not ligated, a circular single-stranded DNA is not formed, and thus RCA reaction does not proceed. In order to perform such an RCA reaction, it is necessary to prepare a single-stranded probe (padlock probe) that can be annealed with genomic DNA and can form a circle.

Specifically, first, there are selected in advance DNA sequences of 10-20 bases starting from or end with the SNP site (121st base) shown in SEQ ID NOs: 1-173 in the Sequence Listing of the present invention. To the 3′ end of the single-stranded probe having a specific sequence as a backbone, the 5′ end of the DNA oligomer having 10-20 bases on the upstream 5′ side of the SNP site is linked. To the 5′ end of the single-stranded probe as a backbone, the 3′ end of the DNA oligomer on the downstream 3′ side of the SNP site is linked. It should be noted that the DNA sequence on the upstream 5′ side of the SNP site used herein matches with the DNA sequence of SEQ ID NOs: 520-692 in the Sequence Listing. As for primers for DNA polymerase in DNA synthesis, RCA primers having a DNA sequence complementary to the probe as a backbone is used.

With this configuration, the padlock probe can be obtained that has the SNP site positioned at the 3′ end when in a circular form. Specifically, when the base at this SNP site is complementary to DNA sample (genomic DNA) to be hybridized with, which was prepared from a cancer patient on whom radiation therapy is to be performed, the DNA probe becomes single-stranded and circular by ligation reaction, and as described above, DNA polymerase synthesizes a long complementary DNA strand by using the circular single-stranded DNA as a template.

On the other hand, when the base at the SNP site is not complementary to genomic DNA to be hybridized with, ligation reaction does not take place and a circular single-stranded DNA is not formed, resulting in no synthesis of a long complementary DNA strand. Therefore, this configuration has an advantage in that whether or not a base is the same as that of the SNP site can be easily determined by simply confirming a presence of long complementary DNA strand by electrophoresis or the like.

It is more preferred that, to the above-mentioned DNA synthetic reaction system, be added a primer having a DNA sequence complementary to a complementary DNA strand synthesized with the DNA polymerase, i.e., DNA sequence identical to the above-mentioned cyclic single-stranded DNA (also called as “branching primer”), since the synthesized DNA has larger molecular weight and confirmation of DNA synthesis becomes easier.

(4-5. SNP Typing Technique-5)

Further, as for other technique for SNP typing to be used in the present invention, there can be mentioned, for example, PCR-SSCP method (single-strand conformation polymorphism) (Genomics, 12, 139-146 (1992); Oncogene, 6, 1313-1318 (1991); PCR Methods Appl, 4, 275-282 (1995)).

Since this method has various advantages, such as relatively easy operation and need for small specimen amount, the method is especially suitable for screening many DNA. samples. The principle is as follows. When a double-stranded DNA fragment is separated into single strands, each strand forms a unique conformation depending on a base sequence thereof. When the separated DNA strands are electrophoresed in polyacrylamide gel containing no denaturant, complementary single-stranded DNAs having the same length move to different positions, depending on their conformational differences. Even one single nucleotide substitution causes conformational change of the single-stranded DNA, resulting in different mobility in polyacrylamide gel electrophoresis. Therefore, if mobility in electrophoresis for various bases at the SNP site is known in advance, SNP typing of single-stranded DNA can be performed by detecting change in mobility.

Specifically, DNA containing a base at a site corresponding to the SNP site shown in SEQ ID NOs: 1-173 in the Sequence Listing of the present invention is amplified by, for example, PCR. In general, a preferable length for amplification is approximately 200-400 bp. For PCR, for example, a cycle may be repeated 30 times, which may include thermal denaturation at 94° C. for 40 seconds, annealing at 50° C. for 1 minute and elongation reaction at 72° C. for 2 minutes. However, the conditions are not limited to these, and can be modified appropriately.

In PCR, the PCR product can be labeled, by using a primer labeled with, such as radioactive isotopes, fluorochromes and biotins as described above. Alternatively, the PCR product may be labeled by adding a substrate base labeled with radioactive isotope, fluorochrome biotin or the like to the PCR reaction solution and conducting PCR. Further, the PCR product may be labeled by adding a substrate base labeled with radioactive isotope, fluorochrome, biotin or the like to a fragment of the PCR product using Klenow enzyme or the like after PCR. For primers used herein, a DNA oligomer set formed of the DNA oligomer of SEQ ID NOs: 174-519 in the Sequence Listing is preferred.

The thus obtained fragment of the labeled PCR product is subjected to thermal denaturation and electrophoresed in polyacrylamide gel containing no denaturant, such as urea. In this case, by adding an appropriate amount (the order of 5-10%) of glycerol to polyacrylamide gel, conditions for separating PCR product fragment can be improved. Migration conditions vary depending on properties of each labeled PCR product, but electrophoresis is generally conducted at room temperature (20-25° C.), and if preferable separation is not obtained, temperature that gives optimum mobility is examined in a range of 4-30° C. After electrophoresis, mobility of the labeled PCR product is analyzed by detecting signals in autoradiography using X-ray film, scanner detecting fluorescence and the like. When a band of the labeled PCR product exhibiting difference in mobility is obtained, the band is directly cut out from the gel, amplified again by PCR, and directly subjected to DNA sequencing, to thereby confirm a presence of variance and determine a type of the base. When the mobility difference of the labeled PCR product according to a base type of the SNP site is known, by comparing with the known mobility, SNP typing can be easily conducted.

(4-6. SNP Typing Technique-6)

Further, as for other techniques for SNP typing to be used in the present invention, there can be mentioned, for example, a method in which restriction fragment length polymorphism (RFLP) is utilized, and PCR-RFLP method.

Specifically, first, a DNA sample was extracted from a cancer patient on whom radiation therapy is to be performed. Regarding a site corresponding to an SNP site shown in SEQ ID NOs: 1-173 in the Sequence Listing of the present invention, when the restriction site is changed by base substitution due to SNP, restriction fragment length is changed. Therefore, SNP is determined by making comparison between a morbidity group and a non-morbidity group, regarding the size of the generated DNA fragment.

In other words, the variation can be detected as mobility difference of bands in electrophoresis, by amplifying the DNA sample containing the variation in PCR, and by treating with corresponding restriction endonuclease.

Alternatively, a presence of variation can be detected by digesting chromosomal DNA with restriction endonuclease, performing electrophoresis, and conducting Southern blotting with a probe containing the DNA oligomer of SEQ ID NOs: 1-173 in the Sequence Listing of the present invention.

It should be noted that the restriction endonuclease used herein can be appropriately selected, depending on types of variation. With this method, other than chromosomal DNA, cDNA can be used. cDNA is obtained, by reverse transcriptase, from RNA prepared from cancer patient on whom radiation therapy is to be performed, and subjected to digestion with restriction endonuclease and to Southern blotting, to thereby determine a presence of variation.

As described above, by detecting a presence of SNP at the restriction site, SNP typing can be made with respect to whether the DNA has a base sequence with likelihood of onset of a side-effect from radiation (morbidity group) or a base sequence with unlikelihood of onset of a side-effect (non-morbidity group).

(4-7. SNP Typing Technique-7)

As described above, the DNA oligomers having DNA sequences of SEQ ID NOs: 1-173 in the Sequence Listing of the present invention and information of the DNA sequences can be used for direct sequencing of genomic DNA, and also used as a probe and the like for DNA chip by synthesizing with, such as a DNA synthesizer. As one embodiment of the DNA oligomer of SEQ ID NOs: 1-173 in the Sequence Listing of the present invention to be used as a probe for DNA chip, a DNA chip for a prediction of onset of a side-effect from radiation therapy can be suitably mentioned.

The expression “DNA chip” used herein means a substrate having a DNA oligomer (probe for DNA chip) arrayed and fixed thereon, which DNA oligomer has a DNA sequence of SEQ ID NOs: 1-173 in the Sequence Listing including an SNP site to be detected. The DNA oligomer on the substrate is hybridized with a target DNA or target RNA labeled with fluorescence, and fluorescent signal on the DNA probe is detected. In the DNA chip, the DNA oligomer fixed on a glass substrate generally serves as a probe, with the target being a labeled DNA in the solution. Therefore, the DNA oligomer fixed on the glass substrate is taken as DNA chip probe.

There are mainly 2 types of DNA chip: Affymetrix Inc. type in which DNA is synthesized on a glass surface; and Stanford type in which cDNA is mounted on a glass surface. In general, Affymetrix Inc. type is considered as suitable for SNP typing, though the present invention is not limited to this type and Stanford type may be used.

Hereinafter, a DNA chip of Affymetrix Inc. type using a DNA oligomer (DNA chip probe) of SEQ ID NOs: 1-173 in the Sequence Listing of the present invention will be explained.

In Affymetrix Inc. type, by using photolithography and light irradiation chemical synthesis in combination, a DNA oligomer of approximately 20-25 bp having an SNP site shown in SEQ ID NOs: 1-173 in the Sequence Listing is synthesized on a glass substrate to thereby obtain a DNA chip having a DNA oligomer of SEQ ID NOs: 1-173 in the Sequence Listing of the present invention fixed thereon. cDNA synthesized from DNA prepared from a sample, or cDNA synthesized through reverse transcription of RNA prepared from a sample, is used as a template. fluorescence-labeled cRNA is synthesized in vitro transcription and hybridized with the DNA chip probe in the present invention under stringent conditions, and fluorescence image is measured with a specific scanner. In this manner, SNP typing can be easily made with respect to whether the DNA has a base sequence with likelihood of onset of a side-effect from radiation (morbidity group) or a base sequence with unlikelihood of onset of a side-effect (non-morbidity group).

There is no limitation with respect to the DNA chip probe fixed on a glass substrate, as long as the DNA chip probe can detect base polymorphism of a target DNA at a site corresponding to the SNP site shown in SEQ ID NOs: 1-173 in the Sequence Listing. In other words, the DNA chip probe does not necessarily have a completely complementary sequence to DNA containing a base at a site corresponding to the SNP site shown in SEQ ID NOs: 1-173 in the Sequence Listing, as long as it can specifically hybridizes with, for example, the target DNA containing a base at a site corresponding to the SNP site shown in SEQ ID NOs: 1-173 in the Sequence Listing, and may include deletion, substitution or insertion of one to several bases. In addition, in the present invention, a length of the DNA chip probe to be fixed on the substrate is preferably 10-100 bp, more preferably 10-50 bp, most preferably 15-25 bp.

A reaction solution and reaction conditions for the hybridization in SNP typing using the DNA chip may vary depending on factors, such as a length of the nucleotide probe fixed on a substrate, which can be determined based on a melting temperature (Tm) of a complex or a target DNA that binds the DNA chip probe. For example, as for washing conditions after hybridization, there can be mentioned stringent conditions, such as “1×SSC, 0.1% SDS, 37° C.”. It is preferred that a complementary strand that can be hybridized with the DNA chip probe fixed on the DNA chip retain a hybridized state with the target DNA, even after being washed under the above-mentioned conditions. For washing conditions, more stringent conditions, such as “0.5×SSC, 0.1% SDS, 42° C.”, further more stringent conditions, such as “0.1×SSC, 0.1% SDS, 65° C.” can be mentioned, though washing conditions are not limited to these. In addition, washing of the hybridized DNA may be conducted with appropriately modifying a concentration of salt, such as NaCl and KCl, and temperature. For example, the salt concentration is selected from 3 M or less, 2.5 M or less, 2 M or less, 1.5 M or less, more preferably from stringent conditions, such as 1 M or less, 0.75 M or less, 0.5 M or less, or even 0.25 M or less, 0.1 M or less. The temperature may be selected from at least approximately 15° C., approximately 20° C., approximately 25° C., approximately 30° C., more preferably from stringent conditions, such as 35° C. or more, 40° C. or more, 45° C. or more, 50° C. ore more, 60° C. or more, 70° C. or more, or even 80° C. or more.

As described above, the DNA oligomer of at least 10 bp containing a base at a site corresponding to the SNP site shown in SEQ ID NOs: 1-173 in the Sequence Listing of the present invention and the complementary DNA oligomer thereto can be used as a probe (or a substrate having the probe fixed thereon) or a primer in SNP typing.

When the DNA oligomer of SEQ ID NOs: 1-173 in the Sequence Listing of the present invention is used as primer, the length is generally 10-241 bp, preferably 15-200 bp, more preferably 15-100 bp, still more preferably 17-50 bp, most preferably 20-30 bp. When the DNA oligomer is used as primer, it is preferred that the DNA oligomer be used as a DNA oligomer set consisting of a pair of DNA oligomers sequentially selected from the DNA oligomers of SEQ ID NOs: 174-519, the sequence identification numbers of the pair starting from even number. However, the present invention is not limited to this embodiment, and any DNA oligomer can be constructed with reference to the DNA sequence of SEQ ID NOs: 1-173 in the Sequence Listing, as long as the DNA oligomer contains the SNP site (the 121st base), which is a risk allele, and is amplifiable. In addition, a DNA oligomer set may be obtained by using sequences outside the above-mentioned sequences on human chromosome.

When the DNA oligomer of SEQ ID NOs: 1-173 in the Sequence Listing is used as probe, there is no limitation with respect to the probe, as long as it can hybridize specifically with DNA containing a base at a site corresponding to the SNP site (121st base) shown in SEQ ID NOs: 1-173 in the Sequence Listing of the present invention. It is preferred that the probe generally have 15 bp or more.

The DNA oligomer of the present invention may be produced preferably by, for example, a commercially available DNA synthesizer. However, the production method is not limited to this, and it may be produced as a double-stranded DNA fragment obtained by, for example, restriction digestion of a vector having the DNA sequence transformed into E. coli and the like.

[5. Radiation Used in Radiation Therapy]

In the present invention, examples of radiation to be used preferably in the radiation therapy include X-ray, gamma-ray, heavy-particle ray and electron beam. However, the radiation is not limited to these, and radiation, such as proton beam and neutron beam, can also be used.

[6. SNP Information]

Information to perform SNP typing is obtained from dbSNP (http://www.ncbi.nlm.nih.gov/SNP/) by NCBI, information of various genes disclosed in various documents, and information registered on JSNP DB (http://snp.ims.u-tokyo.ac.jp/).

[7. A Method for Predicting Onset of a Side-Effect from Radiation Therapy]

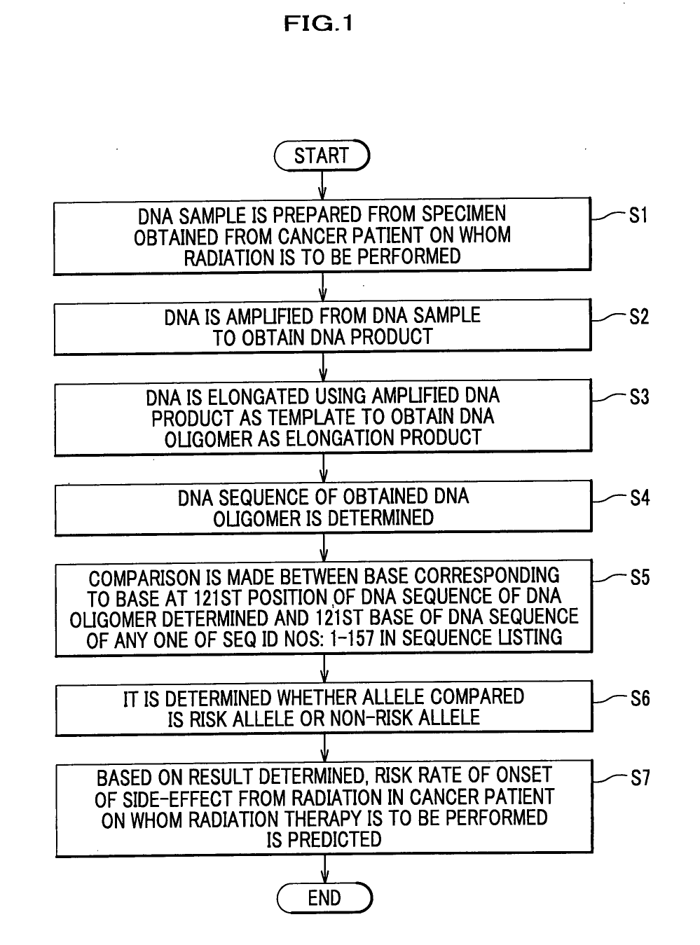

Next, referring to FIG. 1, a method for predicting onset of a side-effect from radiation therapy will be described, in which a DNA sequence of DNA sample prepared from a specimen obtained from a subject (cancer patient) is directly analyzed, and it is determined whether the DNA sequence matches the SNP site (121st base) of the DNA sequence of the DNA oligomer for a prediction of onset of a side-effect from radiation therapy of the present invention. FIG. 1 is a flow chart explaining a method for predicting onset of a side-effect from radiation therapy of the present invention.

The method for predicting onset of a side-effect from radiation therapy of the present invention in which determination is made using the DNA oligomer of SEQ ID NOs: 1-173 in the Sequence Listing includes the following processes (a)-(g):
(a) a DNA sample is prepared from a specimen obtained from a cancer patient on whom radiation therapy is to be performed (step S1);
(b) DNA is amplified from the DNA sample prepared in the process (a) to obtain DNA product (step S2);
(c) elongation reaction is performed using the DNA product amplified in the process (b) as a template, to obtain a DNA oligomer as elongation product (step S3);
(d) a DNA sequence of the DNA oligomer obtained in the process (c) is determined (step S4);
(e) a comparison is made between a base corresponding to a base at a 121st position of the DNA sequence of the DNA oligomer sequenced in the process (d) and a 121st base of the DNA sequence of any one of SEQ ID NOs: 1-173 in the Sequence Listing (step S5);
(f) it is determined whether the allele having the base compared in the process (e) is a risk allele or a non-risk allele (step S6); and
(g) a risk rate of onset of a side-effect from radiation in the cancer patient on whom the radiation therapy is to be performed is predicted, based on the result determined in the process (f) (step S7).

Hereinbelow, each process of the method for predicting onset of a side-effect from radiation therapy of the present invention will be described in detail.

First in the process (a), a DNA sample is prepared from a specimen obtained from a cancer patient on whom radiation therapy is to be performed (step S1) . It is preferred that the specimen be blood. However, the specimen is not limited to this, and any specimen, such as skin, oral mucosa, hair, tissue removed by operation and the like, can be used as long as the specimen contains DNA. By using such a specimen, a sample of DNA, such as chromosomal DNA, can be suitably extracted.

In this process, in order to prepare the DNA sample from blood (specimen) obtained from the cancer patient on whom radiation therapy is to be performed, it is preferred to use an automatic DNA extractor which automatically extracts DNA from blood. This is because a large number of DNA sample can be extracted in a short period of time, and merely easy operation is required. When such an automatic DNA extractor is used, it is preferable to extract DNA using a standard protocol attached to the device, but the protocol may be appropriately modified.

In addition, as other methods for easily extracting DNA sample, various simple systems or kits commercially available can be mentioned.

As still other methods for extracting DNA sample, phenol-chloroform treatment and ethanol precipitation (BASIC METHODS IN MOLECULAR BIOLOGY 2nd EDITION, Davis et al., P. 16-21) can be mentioned. If desired, from a specimen obtained from the cancer patient, a total RNA may be purified (guanidine isothiocyanate-cesium chloride ultracentrifugation; BASIC METHODS IN MOLECULAR BIOLOGY 2nd EDITION, Davis et al., P. 322-328), poly A⁺-RNA may be isolated (BASIC METHODS IN MOLECULAR BIOLOGY 2nd EDITION, Davis et al., P. 344-349), or cDNA may be synthesized (Synthesis of First-Strand cDNA; BASIC METHODS IN MOLECULAR BIOLOGY 2nd EDITION, Davis et al., P. 515-522; id. P. 136-137) based on the above-mentioned purification and isolation. Further, various reagents and kits commercially available can be applied to these operations.

Next in the process (b), DNA is amplified from the DNA sample prepared in the previous process to thereby obtain DNA product (step S2). As a method for amplifying DNA in the DNA sample in this process, PCR is suitably used. For preferable primers to be used in PCR, there can be mentioned a PCR amplification primer set consisting of a pair of DNA oligomers sequentially selected from DNA oligomers of SEQ ID NOs: 174-519 in the Sequence Listing, the SEQ ID NOs of the pair starting from even number. For PCR, for example, thermal denaturation is conducted at 94° C. for 2 minutes, and a cycle may preferably repeated 30 times, which includes: thermal denaturation at 94° C. for 40 seconds; annealing at 50° C. for 1 minute; and elongation reaction at 72° C. for 2 minutes. Then, final elongation reaction is further conducted at 72° C. for 5 minutes. However, the conditions are not limited to these, and can be modified appropriately. Moreover, DNA may be amplified using, for example, DNA polymerase, in a case where the DNA product can be amplified without using PCR.

Next in the process (c), elongation reaction is performed using the DNA product amplified in the previous process as a template, and a DNA oligomer as elongation product is obtained (step S3). For the extension primer to be used for DNA sequence analysis, DNA oligomers of SEQ ID NOs: 520-692 in the Sequence Listing of the present invention are preferably used. Conditions for elongation reaction may be appropriately set or modified, but it is preferred that the conditions be those designated in a protocol of a kit for elongation.

Next in the process (d), DNA sequence of the DNA oligomer is determined, which was subjected to elongation reaction in the previous process (step S4).

Herein, as a method for analyzing the DNA sequence of the DNA product, MALDI-TOF/MS method described above in detail is preferably used which can directly analyze the DNA sequence. However, the method is not limited to this, and DNA sequence analysis, i.e., SNP typing can be performed using the above-mentioned appropriate techniques.

Next in the process (e), a base corresponding to a base at the 121st position of the DNA sequence of the DNA oligomer analyzed in the previous process is compared with the 121st base of the DNA sequence of SEQ ID NOs: 1-173 in the Sequence Listing (step S5).

In the process (f), it is determined whether the allele having the base compared in the previous process is a risk allele or a non-risk allele (step S6).

In the next process (g), a risk rate of onset of a side-effect from radiation in the cancer patient on whom the radiation therapy is to be performed is predicted, by taking factors into account, such as: the result of the determination whether the base of the DNA product equivalent to the base at the 121st position of the DNA sequence of SEQ ID NOs: 1-173 in the Sequence Listing is a risk allele or a non-risk allele; and allele frequency at other SNP site (step S7).

The DNA oligomer, the genetic marker, the DNA oligomer set for a prediction of onset of a side-effect and the method for predicting onset of a side-effect from radiation therapy of the present invention are described in detail. However, the present invention should not be limited to the particular embodiments discussed above and may be carried out in various modified forms without departing the spirit of the present invention.

For example, (1) to the genetic marker for a prediction of onset of a side-effect from radiation therapy of the present invention, at least one of fluorochrome, radioactive isotope, enzyme for fluorochrome emission, and protein with binding ability with a specific substance may be added.

For example, (2) the genetic marker for a prediction of onset of a side-effect from radiation therapy of the present invention may be those obtained by adding different fluorochromes, radioactive isotopes, enzymes for fluorochrome emission, or proteins with binding ability with a specific substance, to a DNA oligomer having a DNA sequence complementary to a risk allele and to a DNA oligomer having a DNA sequence complementary to a non-risk allele.

The DNA oligomer of the present invention to be used for detection may be those obtained by adding fluorochrome, radioactive isotope, enzyme for fluorochrome emission or protein with binding ability with a specific substance, to the DNA oligomer according to any one of [1]-[16] and [20] defined as aspects of the present invention, or one of the DNA oligomers of the DNA oligomer set according to [18] or [19].

For example, (3) the DNA oligomer of the present invention to be used for detection may be those obtained by adding different fluorochromes, radioactive isotopes, enzymes for fluorochrome emission, or proteins with binding ability with a specific substance, to a DNA oligomer having a DNA sequence complementary to a risk allele and to a DNA oligomer having a DNA sequence complementary to a non-risk allele.

For example, (4) the method for predicting onset of a side-effect from radiation therapy of the present invention may be a method including the following processes (a)-(e), wherein determination is made using the DNA oligomer of SEQ ID NOs: 520-692 in the Sequence Listing: (a) a DNA sample is prepared from a specimen obtained from a cancer patient on whom radiation therapy is to be performed; (b) the DNA sample prepared in the process (a) and a DNA oligomer having a DNA sequence of any of SEQ ID NOs: 520-692 in the Sequence Listing are hybridized; (c) the DNA oligomer hybridized in the process (b) is allele-specifically elongated by 1 base using ddNTP; (d) base of ddNTP for the DNA oligomer which was allele-specifically elongated by 1 base in the process (c) is analyzed; (e) the base of ddNTP analyzed in the process (d) is compared with the 121st base of the DNA oligomer of any one of SEQ ID NOs: 1-157 in the Sequence Listing; (f) it is determined whether the allele having the base compared in the process (e) is a risk allele or a non-risk allele; and (g) a risk rate of onset of a side-effect from radiation in the cancer patient on whom the radiation therapy is to be performed is predicted, based on the result determined in the process (f).

Finally, (5) the ddNTP may be those to which fluorochrome, radioactive isotope, enzyme for fluorochrome emission, or protein with binding ability with a specific substance, is added.

### EXAMPLE

Next, the DNA oligomer, genetic marker, DNA oligomer set (PCR primer) and DNA oligomer (extension primer) for a prediction of onset of a side-effect, and the method for predicting onset of a side-effect from radiation therapy of the present invention will be further described with reference to Examples.

For study of SNP for predicting a risk rate of onset of a side-effect, the followings are conducted: (1) analysis of radiosensitivity gene in cultivated human cell lines; (2) analysis of radiosensitivity gene associated with difference in mouse strain; (3) document research; and (4) analysis in view of genetic statistics regarding onset of a side-effect from radiation.

[1] Radiosensitivity gene in cultivated human cell lines. Various reports have been made with respect to genes associated with radiosensitivity. Those reports focus on genes having specific functions, such as certain DNA-repair genes. In the present invention, studies were made not only on these genes, but also on 21,000 kinds of genes or gene candidates selected based on the analysis of DNA sequence information utilizing all DNA sequences of human genome that have been recently sequenced. Production of oligoarray having a DNA sequence having 60 bases with genetically specificity was entrusted to Agilent Technologies Inc. (CA, USA), and search was conducted for genes useful for categorizing cell lines in association with radiosensitivity.

First, dose-survival curves for each of 60 different cultivated human cell lines were obtained, and 32 cell lines having different sensitivities were selected. Gene expression profiles before and after X-ray radiation were compared between cells, and genes useful for categorizing cell lines were identified by parameters of models, such as D₁₀, D_{q} and α/β, and taken as radiosensitivity gene candidates. These include genes considered to be associated with cell proliferation, cell cycle regulation, redox and DNA damage repair.

[2] Analysis associated with difference in mouse strain. With respect to 3 strains of mouse including A/J, C3H/HeMs and C57BL6J that have different radiosensitivity, damage/repair process after radiation in various organs, such as skin, lung and intestine, was examined (M. Iwakawa et al., Radiation Res., 44, 7-13 (2003)). At the same time, gene expression profiles in an organ of interest after radiation were compared using microarray, and gene cluster considered to be associated with difference in strain was extracted. Human homologs of these genes were taken as candidates for susceptibility gene. These include genes related to signal transduction, apoptosis and immunity.

[3] Analysis in view of genetic statistics regarding onset of a side-effect from radiation. DNA sequence information related to polymorphism marker (SNP site) on a candidate gene was obtained from UCSC Genome Bioinformatics (version: UCSC Human April 2003 (http://genome.ucsc.edu/)), JSNP DB (http://snp.ims.u-tokyo.ac.jp/) and dbSNP (http://www.ncbi.nlm.nih.gov/SNP/). Specimens of a group of healthy subject (group of non-morbidity with SNP allele) collected by the present inventors were used for analysis of polymorphism frequency of these alleles, and alleles to be analyzed were selected.

Next, with respect to groups categorized by determination of onset of a side-effect from radiation, SNP typing was conducted. Subjects of the study were: 218 breast cancer patients, 57 cervical cancer patients, and 71 prostate cancer patients, who offered blood and clinical record after receiving informed consent regarding the study, between October 2001 and December 2003. First medical data was analyzed, and stratified for polymorphism frequency analysis. With respect to a side-effect (disorder) from radiation, occurrence of dermatitis, intestinal disorder (diarrhea) and vesical dysfunction or ureteropathy (urination disorder) were monitored for breast cancer patient, cervical cancer patient and prostate cancer patient, respectively. For each disorder, data were classified into 2 groups, based on results obtained during a period from a beginning of the radiation therapy to at less than 3 months (early stage), a period of 3 months or after (late stage of 3 months) or a period to 6 months or after (late stage of 6 months).

Each of Tables 1-18 shows allele frequencies statistically obtained from breast cancer patients, cervical cancer patients or prostate cancer patients during an early stage, a late stage of 3 months (not shown with respect to cervical cancer), and a late stage of 6 months from a beginning of the therapy, who offered informed consent regarding DNA sampling and SNP typing. Each of Talbes shows a type of cancer, a period when a side-effect from radiation therapy (disorder) was observed, and a studied body part.

In other words, each of Tables 1-3 shows allele frequency of breast cancer patient group during a period from a beginning of the radiation therapy for breast cancer to an early stage; each of Tables 4 and 5 shows allele frequency of breast cancer patient group during a late stage of 3 months from a beginning of the therapy for breast cancer; each of Tables 6 and 7 shows allele frequency of breast cancer patient group during a late stage of 6 months from a beginning of the therapy for breast cancer; each of Tables 8-11 shows allele frequency of cervical cancer patient group during a period from a beginning of the radiation therapy for cervical cancer to an early stage; Table 12 shows allele frequency of cervical cancer patient group during a late stage of 6 months from a beginning of the therapy for cervical cancer; each of Tables 13 and 14 shows allele frequency of prostate cancer patient group during a period from a beginning of the radiation therapy for prostate cancer to an early stage; each of Tables 15 and 16 shows allele frequency of prostate cancer patient group during a late stage of 3 months from a beginning of the therapy for prostate cancer; and each of Tables 17 and 18 shows allele frequency of prostate cancer patient group during a late stage of 6 months from a beginning of the therapy for prostate cancer.

Columns A-J in Tables 1-18 show the following contents.

Column A shows SNP ID and genotype allotted to each SNP and registered at public data bank (e.g., NCBI and JSNP (IMS-JST SNP)), i.e. identified allele frequency. With respect to breast cancer, cervical cancer and prostate cancer, dermatitis, intestinal disorder, and ureteropathy were studied, respectively.

Regarding the genotype shown in the column, upper allele(s) indicates risk allele, while lower allele(s) indicate non-risk allele. Herein, C/C and G/G, for example, means that an allele is a homozygote with C (cytosine) and C (cytosine) and a homozygote with G (guanine) and G (guanine), respectively, and C/G means that the allele is a heterozygote with C (cytosine) and G (guanine).

With respect to a time period in which a side-effect from radiation therapy is confirmed, the term “early stage” means that disorder was observed in less than 3 months after the radiation therapy, “late stage of 3 months” means that disorder was observed between 3 months and 6 months after the radiation therapy, and “late stage of 6 months” means that disorder was observed 6 months or more after the radiation therapy.

It should be noted that a relationship between normal allele and associated risk allele may be opposite, depending on disorder to which an attention is paid. For example, in a case of a site of rs2561829 shown in Table 1, C/C allele is a risk allele in an early stage after radiation therapy for breast cancer, while in the case of prostate cancer shown in Table 17, T/T allele and T/C allele are risk alleles in a late stage of 6 months after radiation therapy. Since gene function analysis has not been completed at present, it is difficult to give explanation for such an opposite relationship, but statistical analysis demonstrates this result. This may be because a vector (i.e., direction of transcription and translation of gene) is reversed depending on functions of expressed genes or proteins, body part or period of onset and the like, or balance in expression level of alleles has an effect on difference in a risk allele and a non-risk allele. For such an SNP site, in addition to the above-mentioned rs2561829, there can be mentioned rs4818 (see Tables 3 and 4), rs791041 (see Tables 2 and 17) , rs704227 (see Tables 12 and 15) , rs2283264 (see Tables 2 and 17) , rs73234 (see Tables 2, 9 and 14), rs518116 (see Tables 3 and 12) , rs1171097 (see Tables 1 and 15), rs791040 (see Tables 2 and 17) , rs1145720 (see Tables 3 and 18), rs1144153 (see Tables 2 and 17), rs2267437 (see Tables 1, 8 and 13), rs2270390 (see Tables 12 and 16), and rs2072817 (see Tables 7 and 8).

Column B shows a case number (n) with which disorder was observed after radiation therapy and grades (1-4) thereof.

Column C shows a case number (n) with which either with no disorder (Grade 0) or light disorder (Grade 1).

With respect to the level of a side-effect indicated with grade 0, 1, 2, 3 or 4, a larger numeral indicates a more severe case. A side-effect in an early stage was determined according to NCI/CTC (National Cancer Institute, Common Toxicity Criteria), which is an international determination standard. A side-effect (disorder) at late stages of 3 months and 6 months from a beginning of the radiation therapy were determined according to RTOG (Radiation Therapy Oncology Group), which is an international determination standard. From the obtained clinical information, items were selected depending on the type of cancer. For example, in a case of breast cancer, 38 items were selected regarding age, smoking, drinking, complication, clinical history of family member, TNM classification, pathology examination, chemotherapy, radiation method, recrudescence, metastatis and the like. The selected items were analyzed and standardized, and cases which do not meet conditions were excluded from polymorphism frequency analysis.

Column D shows P value according to Fisher's test (exact probability). In biostatistics, P value of 0.05 or less is considered to have a significant difference. In the present invention, a degree of freedom is set to 1 in all cases.

Column E shows relative risk. The relative risk is also called as risk ratio or risk rate, and is obtained by dividing a risk of onset of a side-effect (disorder) in a case where there is a factor [determined as radiosensitive (i.e., with a risk of onset of a side-effect) based on determination with each risk allele or a combination of risk alleles], by a risk of onset of a side-effect in a case where there is no factor [determined as non-radiosensitive (i.e., without a risk of onset of a side-effect) with almost no onset of a side-effect]. A higher value of the relative risk indicates that the allele is more likely to have onset of a side-effect.

Column F shows 95% confidence interval. In each Table, there are some cases in which the relative risk or the 95% confidence interval were not obtained (in Tables 1 to 18, shown with “−”), since the allele frequency of the sample was “0”.

Column G shows sequence identification number of DNA oligomer for a prediction of onset of aside-effect from radiation therapy of the present invention having a risk allele.

Column H shows sequence identification number of DNA oligomer (forward primer) used in PCR.

Column I shows sequence identification number of DNA oligomer (reverse primer) used in PCR.

It should be noted that, for each SNP site, a DNA oligomer set formed of sequence identification numbers shown in Columns H and I can be used as a PCR amplification primer set.

Column J shows sequence identification number of DNA oligomer (extension primer) used in SNP typing. It should be noted that, the extension primers of the present invention include DNA sequences each with a strand in the same direction as those of DNA oligomer of SEQ ID NOs: 1-173, as well as DNA sequences each with a strand in the opposite direction to the above. In a case of an extension primer with a reverse strand, a base complementary to the base added to downstream 3′ side of the extension primer corresponds to the 121st base (risk allele) shown in any one of SEQ ID NOs: 1-173.

For example, referring to SNP ID: rs1171097 shown at the top of Table 1, it is shown that disorder was observed on skin of breast cancer patient at an early stage (less than 3 months) from the beginning of the radiation therapy, and the genotype thereof (designated as “genotype” in Column A of Tables 1-18) shows C/C homozygote as a risk allele. It should be note that, in this SNP, G/G homozygote and C/G heterozygote are non-risk alleles (Column A).

Specifically, the number of breast cancer patients with which skin disorder was observed (Grade 1, 2, 3) during a period from a beginning of the radiation therapy to an early stage was 135 (Column B), and the number of breast cancer patients with which skin disorder was not observed (Grade 0) was 12 (Column C).

In a case of breast cancer with skin disorder, the number of patients having C/C homozygote was 88, while in a case of breast cancer without skin disorder, the number of patients having G/G homozygote or C/G heterozygote was 47 (Column B).

In a case of breast cancer without skin disorder, the number of patients having C/C homozygote was 3, while in a case of breast cancer without skin disorder, the number of patients having G/G homozygote or C/G heterozygote was 9 (Column C).

Statistical analysis of these results with Fisher's test (exact probability) shows that, in the case of breast cancer, there was a significant difference (P value=0.01041) between the patients with C/C homozygote and the patients with G/G homozygote or C/G heterozygote (Column D) . A relative risk of the risk allele was 1.15 (Column E), and a 95% confidence interval was 1.02-1.30 (Column F).

SEQ ID NO: 17 is a DNA sequence of the DNA oligomer having the risk allele (Column G), and SEQ ID NO: 206 is the forward primer (Column H) and SEQ ID NO: 207 is the reverse primer (Column I), of the PCR amplification primers used for amplification of the DNA sequence. SEQ ID NO: 487 is the extension primer used for determining DNA base of the SNP site (risk allele) (Column J).

The illustrative description has been made for a table shown in the present invention, and the similar description can be applied to other SNP shown in Tables 1 to 18.

TABLE 1
Breast cancer, early stage, dermatitis

| | | | | | | | G | H | I | J |
|---|---|---|---|---|---|---|---|---|---|---|
| | | C | D | E | F | | SEQ. | SEQ. | SEQ. | SEQ. |
| | | Grade 0 | Fisher | Relative | [95% confidence | | ID. | ID. | ID. | ID. |
| A | B | n = 12 | (p value) | risk | interval] | | NO. | NO. | NO. | NO. |
| rs1171097 | Grade 1, 2, 3 | | | | | | | | | |
| Genotype | n = 135 | | | | | | | | | |
| C/C | 88 | 3 | 0.01041 | 1.15 | 1.02 | 1.30 | 17 | 206 | 207 | 536 |
| C/G or G/G | 47 | 9 | | | | | | | | |
| rs565435 | Grade 1, 2, 3 | | | | | | | | | |
| Genotype | n = 134 | | | | | | | | | |
| C/C | 83 | 2 | 0.00399 | 1.17 | 1.04 | 1.31 | 140 | 452 | 453 | 659 |
| C/G or G/G | 51 | 10 | | | | | | | | |
| rs818707 | Grade 1, 2, 3 | | | | | | | | | |
| Genotype | n = 135 | | | | | | | | | |
| C/C or C/T | 135 | 10 | 0.00615 | — | — | — | 167 | 506 | 507 | 686 |
| T/T | 0 | 2 | | | | | | | | |
| rs3735048 | Grade 1, 2, 3 | | | | | | | | | |
| Genotype | n = 135 | | | | | | | | | |
| G/G or C/G | 124 | 8 | 0.02165 | 1.28 | 0.94 | 1.74 | 112 | 396 | 397 | 631 |
| C/C | 11 | 4 | | | | | | | | |
| rs1055677 | Grade 1, 2, 3 | | | | | | | | | |
| Genotype | n = 134 | | | | | | | | | |
| C/C | 107 | 6 | 0.02841 | 1.16 | 0.98 | 1.37 | 7 | 186 | 187 | 526 |
| C/G or G/G | 27 | 6 | | | | | | | | |
| rs2866635 | Grade 1, 2, 3 | | | | | | | | | |
| Genotype | n = 135 | | | | | | | | | |
| G/G | 70 | 2 | 0.03154 | 1.12 | 1.02 | 1.24 | 106 | 384 | 385 | 625 |
| G/A or A/A | 65 | 10 | | | | | | | | |
| rs2267437 | Grade 1, 2, 3 | | | | | | | | | |
| Genotype | n = 135 | | | | | | | | | |
| C/C | 54 | 1 | 0.03172 | 1.12 | 1.03 | 1.21 | 65 | 302 | 303 | 584 |
| C/G or G/G | 81 | 11 | | | | | | | | |
| rs5938 | Grade 1, 2, 3 | | | | | | | | | |
| Genotype | n = 135 | | | | | | | | | |
| C/C | 107 | 6 | 0.03196 | 1.15 | 0.98 | 1.35 | 143 | 458 | 459 | 662 |
| C/A or A/A | 28 | 6 | | | | | | | | |
| rs1555025 | Grade 1, 2, 3 | | | | | | | | | |
| Genotype | n = 135 | | | | | | | | | |
| G/G or G/T | 133 | 10 | 0.03359 | 1.86 | 0.70 | 4.96 | 27 | 226 | 227 | 546 |
| T/T | 2 | 2 | | | | | | | | |
| rs2561829 | Grade 1, 2, 3 | | | | | | | | | |
| Genotype | n = 135 | | | | | | | | | |
| C/C | 85 | 3 | 0.01381 | 1.14 | 1.02 | 1.28 | 94 | 360 | 361 | 613 |
| C/T or T/T | 50 | 9 | | | | | | | | |
| rs6267 | Grade 1, 2, 3 | | | | | | | | | |
| Genotype | n = 135 | | | | | | | | | |
| G/G | 119 | 7 | 0.01504 | 1.24 | 0.97 | 1.58 | 147 | 466 | 467 | 666 |
| G/T or T/T | 16 | 5 | | | | | | | | |
| rs3825609 | Grade 1, 2, 3 | | | | | | | | | |
| Genotype | n = 135 | | | | | | | | | |
| G/G | 96 | 4 | 0.01889 | 1.16 | 1.01 | 1.32 | 127 | 426 | 427 | 646 |
| G/C or C/C | 39 | 8 | | | | | | | | |

TABLE 2
Breast cancer, early stage, dermatitis

| | | | D | | | | G | H | I | J |
|---|---|---|---|---|---|---|---|---|---|---|
| | | | Fisher | E | F | | SEQ. | SEQ. | SEQ. | SEQ. |
| | | | (p | Relative | [95% confidence | | ID. | ID. | ID. | ID. |
| A | B | C | value) | risk | interval] | | NO. | NO. | NO. | NO. |
| rs1339458 | Grade 1, 2, 3 | Grade 0 | | | | | | | | |
| Genotype | n = 135 | n = 12 | | | | | | | | |
| T/T or T/C | 67 | 2 | 0.0351 | 1.11 | 1.01 | 1.22 | 20 | 212 | 213 | 539 |
| C/C | 68 | 10 | | | | | | | | |
| rs2007182 | Grade 2, 3 | Grade 0, 1 | | | | | | | | |
| Genotype | n = 40 | n = 107 | | | | | | | | |
| G/G | 38 | 86 | 0.03923 | 3.52 | 0.91 | 13.60 | 45 | 262 | 263 | 564 |
| G/A or A/A | 2 | 21 | | | | | | | | |
| rs1971783 | Grade 2, 3 | Grade 0, 1 | | | | | | | | |
| Genotype | n = 40 | n = 107 | | | | | | | | |
| T/T | 23 | 38 | 0.02344 | 1.91 | 1.12 | 3.25 | 44 | 260 | 261 | 563 |
| T/C or C/C | 17 | 69 | | | | | | | | |
| rs2239393 | Grade 2, 3 | Grade 0, 1 | | | | | | | | |
| Genotype | n = 40 | n = 107 | | | | | | | | |
| G/G | 5 | 1 | 0.00587 | 3.36 | 2.12 | 5.31 | 61 | 294 | 295 | 580 |
| G/A or A/A | 35 | 106 | | | | | | | | |
| rs791040 | Grade 2, 3 | Grade 0, 1 | | | | | | | | |
| Genotype | n = 40 | n = 107 | | | | | | | | |
| A/A or A/G | 35 | 75 | 0.03355 | 2.35 | 1.00 | 5.56 | 163 | 498 | 499 | 682 |
| G/G | 5 | 32 | | | | | | | | |
| rs791041 | Grade 2, 3 | Grade 0, 1 | | | | | | | | |
| Genotype | n = 40 | n = 107 | | | | | | | | |
| A/A or A/T | 35 | 75 | 0.03355 | 2.35 | 1.00 | 5.56 | 165 | 502 | 503 | 684 |
| T/T | 5 | 32 | | | | | | | | |
| rs1144153 | Grade 2, 3 | Grade 0, 1 | | | | | | | | |
| Genotype | n = 40 | n = 107 | | | | | | | | |
| C/C or C/T | 35 | 75 | 0.03355 | 2.35 | 1.00 | 5.56 | 13 | 198 | 199 | 532 |
| T/T | 5 | 32 | | | | | | | | |
| rs373759 | Grade 2, 3, 4 | Grade 0, 1 | | | | | | | | |
| Genotype | n = 40 | n = 107 | | | | | | | | |
| T/T | 15 | 21 | 0.03174 | 1.85 | 1.10 | 3.11 | 113 | 398 | 399 | 632 |
| T/C or C/C | 25 | 86 | | | | | | | | |
| rs2283264 | Grade 2, 3 | Grade 0, 1 | | | | | | | | |
| Genotype | n = 40 | n = 107 | | | | | | | | |
| G/G or G/C | 11 | 10 | 0.00812 | 2.28 | 1.36 | 3.82 | 78 | 328 | 329 | 597 |
| C/C | 29 | 97 | | | | | | | | |
| rs170548 | Grade 2, 3, 4 | Grade 0, 1 | | | | | | | | |
| Genotype | n = 40 | n = 107 | | | | | | | | |
| A/A or A/C | 25 | 86 | 0.03174 | 1.85 | 1.10 | 3.11 | 32 | 236 | 237 | 551 |
| C/C | 15 | 21 | | | | | | | | |
| rs73234 | Grade 2, 3 | Grade 0, 1 | | | | | | | | |
| Genotype | n = 34 | n = 98 | | | | | | | | |
| G/G or G/C | 23 | 46 | 0.04667 | 1.91 | 1.01 | 3.59 | 157 | 486 | 487 | 676 |
| C/C | 11 | 52 | | | | | | | | |
| rs767298 | Grade 2, 3 | Grade 0, 1 | | | | | | | | |
| Genotype | n = 39 | n = 106 | | | | | | | | |
| T/T | 38 | 90 | 0.04193 | 5.05 | 0.74 | 34.43 | 162 | 496 | 497 | 681 |
| T/G or G/G | 1 | 16 | | | | | | | | |

TABLE 3
Breast cancer, early stage, dermatitis

| | | | | | | | G | H | I | J |
|---|---|---|---|---|---|---|---|---|---|---|
| | | | D | E | F | | SEQ. | SEQ. | SEQ. | SEQ. |
| | | | Fisher | Relative | [95% confidence | | ID. | ID. | ID. | ID. |
| A | B | C | (p value) | risk | interval] | | NO. | NO. | NO. | NO. |
| rs750621 | Grade 2, 3 | Grade 0, 1 | | | | | | | | |
| Genotype | n = 40 | n = 107 | | | | | | | | |
| G/G | 38 | 86 | 0.03923 | 3.52 | 0.91 | 13.60 | 160 | 492 | 493 | 679 |
| G/T or T/T | 2 | 21 | | | | | | | | |
| rs4818 | Grade 2, 3 | Grade 0, 1 | | | | | | | | |
| Genotype | n = 40 | n = 107 | | | | | | | | |
| G/G | 7 | 1 | 0.00047 | 3.69 | 2.48 | 5.48 | 132 | 436 | 437 | 651 |
| G/C or C/C | 33 | 106 | | | | | | | | |
| rs1145720 | Grade 2, 3 | Grade 0, 1 | | | | | | | | |
| Genotype | n = 39 | n = 105 | | | | | | | | |
| T/T or T/C | 34 | 73 | 0.03328 | 2.35 | 0.99 | 5.56 | 15 | 202 | 203 | 534 |
| C/C | 5 | 32 | | | | | | | | |
| rs2232242 | Grade 2, 3 | Grade 0, 1 | | | | | | | | |
| Genotype | n = 40 | n = 106 | | | | | | | | |
| A/A | 35 | 73 | 0.03308 | 2.46 | 1.04 | 5.83 | 59 | 290 | 291 | 578 |
| A/G or G/G | 5 | 33 | | | | | | | | |
| rs2705 | Grade 2, 3 | Grade 0, 1 | | | | | | | | |
| Genotype | n = 39 | n = 107 | | | | | | | | |
| T/T or T/C | 27 | 50 | 0.0239 | 2.02 | 1.11 | 3.66 | 98 | 368 | 369 | 617 |
| C/C | 12 | 57 | | | | | | | | |
| rs243387 | Grade 2, 3 | Grade 0, 1 | | | | | | | | |
| Genotype | n = 39 | n = 107 | | | | | | | | |
| T/T or T/C | 27 | 50 | 0.0239 | 2.02 | 1.11 | 3.66 | 91 | 354 | 355 | 610 |
| C/C | 12 | 57 | | | | | | | | |
| rs243336 | Grade 2, 3 | Grade 0, 1 | | | | | | | | |
| Genotype | n = 40 | n = 107 | | | | | | | | |
| C/C or C/G | 29 | 57 | 0.03982 | 1.87 | 1.01 | 3.45 | 90 | 352 | 353 | 609 |
| G/G | 11 | 50 | | | | | | | | |
| rs13385 | Grade 2, 3 | Grade 0, 1 | | | | | | | | |
| Genotype | n = 40 | n = 107 | | | | | | | | |
| G/G or G/A | 38 | 85 | 0.024 | 3.71 | 0.96 | 14.34 | 19 | 210 | 211 | 538 |
| A/A | 2 | 22 | | | | | | | | |
| rs3750496 | Grade 2, 3 | Grade 0, 1 | | | | | | | | |
| Genotype | n = 40 | n = 107 | | | | | | | | |
| A/A | 40 | 96 | 0.03577 | — | — | — | 117 | 406 | 407 | 636 |
| A/G or G/G | 0 | 11 | | | | | | | | |
| rs518116 | Grade 2, 3 | Grade 0, 1 | | | | | | | | |
| Genotype | n = 39 | n = 107 | | | | | | | | |
| A/A | 5 | 2 | 0.01497 | 2.92 | 1.68 | 5.07 | 138 | 448 | 449 | 657 |
| A/G or G/G | 34 | 105 | | | | | | | | |
| rs2272615 | Grade 2, 3 | Grade 0, 1 | | | | | | | | |
| Genotype | n = 40 | n = 107 | | | | | | | | |
| G/G or G/A | 18 | 29 | 0.04756 | 1.74 | 1.04 | 2.92 | 73 | 318 | 319 | 592 |
| A/A | 22 | 78 | | | | | | | | |
| rs153017 | Grade 2, 3 | Grade 0, 1 | | | | | | | | |
| Genotype | n = 40 | n = 106 | | | | | | | | |
| A/A | 24 | 42 | 0.03965 | 1.82 | 1.06 | 3.13 | 26 | 224 | 225 | 545 |
| A/G or G/G | 16 | 64 | | | | | | | | |
| rs4983548 | Grade 2, 3 | Grade 0, 1 | | | | | | | | |
| Genotype | n = 40 | n = 107 | | | | | | | | |
| C/C | 36 | 75 | 0.01661 | 2.92 | 1.12 | 7.64 | 137 | 446 | 447 | 656 |
| C/T or T/T | 4 | 32 | | | | | | | | |

TABLE 4
Breast cancer, late stage of 3 months, dermatitis

| | | | | | | | G | H | I | J |
|---|---|---|---|---|---|---|---|---|---|---|
| | B | | D | E | F | | SEQ. | SEQ. | SEQ. | SEQ. |
| | Grade 1, 2, 3 | | Fisher | Relative | [95% confidence | | ID. | ID. | ID. | ID. |
| A | n = 45 | C | (p value) | risk | interval] | | NO. | NO. | NO. | NO. |
| rs4818 | | Grade 0 | | | | | | | | |
| Genotype | | n = 99 | | | | | | | | |
| C/C | 31 | 48 | 0.02992 | 1.82 | 1.06 | 3.12 | 133 | 438 | 439 | 652 |
| C/G or G/G | 14 | 51 | | | | | | | | |
| rs2229688 | | Grade 0 | | | | | | | | |
| Genotype | | n = 99 | | | | | | | | |
| A/A or A/G | 11 | 11 | 0.04782 | 1.79 | 1.08 | 2.98 | 58 | 288 | 289 | 577 |
| G/G | 34 | 88 | | | | | | | | |
| rs3809454 | | Grade 0 | | | | | | | | |
| Genotype | | n = 99 | | | | | | | | |
| C/C | 8 | 6 | 0.03623 | 2.01 | 1.18 | 3.41 | 126 | 424 | 425 | 645 |
| C/A or A/A | 37 | 93 | | | | | | | | |
| rs615942 | | Grade 0 | | | | | | | | |
| Genotype | | n = 99 | | | | | | | | |
| T/T | 16 | 16 | 0.01625 | 1.93 | 1.21 | 3.08 | 145 | 462 | 463 | 664 |
| T/G or G/G | 29 | 83 | | | | | | | | |
| rs632758 | | Grade 0 | | | | | | | | |
| Genotype | | n = 99 | | | | | | | | |
| T/T | 16 | 16 | 0.01625 | 1.93 | 1.21 | 3.08 | 148 | 468 | 469 | 667 |
| T/G or G/G | 29 | 83 | | | | | | | | |
| rs3087386 | | Grade 0 | | | | | | | | |
| Genotype | | n = 99 | | | | | | | | |
| A/A or A/G | 44 | 85 | 0.03738 | 5.12 | 0.76 | 34.50 | 108 | 388 | 389 | 627 |
| G/G | 1 | 14 | | | | | | | | |
| rs767298 | | Grade 0 | | | | | | | | |
| Genotype | | n = 97 | | | | | | | | |
| T/T | 44 | 82 | 0.0215 | 5.59 | 0.83 | 37.83 | 162 | 496 | 497 | 681 |
| T/G or G/G | 1 | 15 | | | | | | | | |
| rs2071010 | | Grade 0 | | | | | | | | |
| Genotype | | n = 99 | | | | | | | | |
| A/A | 5 | 0 | 0.00254 | — | — | — | 49 | 270 | 271 | 568 |
| A/G or G/G | 40 | 99 | | | | | | | | |

TABLE 5
Breast cancer, late stage of 3 months, dermatitis

| | | | | | | | G | H | I | J |
|---|---|---|---|---|---|---|---|---|---|---|
| | | | D | E | F | | SEQ. | SEQ. | SEQ. | SEQ. |
| | | | Fisher | Relative | [95% confidence | | ID. | ID. | ID. | ID. |
| A | B | C | (p value) | risk | interval] | | NO. | NO. | NO. | NO. |
| rs2276048 | Grade 1, 2, 3 | Grade 0 | | | | | | | | |
| Genotype | n = 45 | n = 99 | | | | | | | | |
| G/G | 6 | 3 | 0.02674 | 2.31 | 1.36 | 3.93 | 77 | 326 | 327 | 596 |
| G/A or A/A | 39 | 96 | | | | | | | | |
| rs2066505 | Grade 1, 2, 3 | Grade 0 | | | | | | | | |
| Genotype | n = 45 | n = 98 | | | | | | | | |
| A/A or A/G | 18 | 20 | 0.02388 | 1.84 | 1.15 | 2.94 | 48 | 268 | 269 | 567 |
| G/G | 27 | 78 | | | | | | | | |
| rs740059 | Grade 1, 2, 3 | Grade 0 | | | | | | | | |
| Genotype | n = 45 | n = 99 | | | | | | | | |
| G/G or G/A | 23 | 30 | 0.0247 | 1.80 | 1.11 | 2.89 | 159 | 490 | 491 | 678 |
| A/A | 22 | 69 | | | | | | | | |
| rs651646 | Grade 1, 2, 3 | Grade 0 | | | | | | | | |
| Genotype | n = 45 | n = 99 | | | | | | | | |
| A/A | 11 | 9 | 0.01918 | 2.01 | 1.23 | 3.27 | 151 | 474 | 475 | 670 |
| A/T or T/T | 34 | 90 | | | | | | | | |
| rs9110 | Grade 1, 2, 3 | Grade 0 | | | | | | | | |
| Genotype | n = 45 | n = 99 | | | | | | | | |
| A/A | 8 | 6 | 0.03623 | 2.01 | 1.18 | 3.41 | 170 | 512 | 513 | 689 |
| A/G or G/G | 37 | 93 | | | | | | | | |
| rs2073495 | Grade 1, 2, 3 | Grade 0 | | | | | | | | |
| Genotype | n = 45 | n = 99 | | | | | | | | |
| G/G | 8 | 6 | 0.03623 | 2.01 | 1.18 | 3.41 | 53 | 278 | 279 | 572 |
| G/C or C/C | 37 | 93 | | | | | | | | |
| rs3744357 | Grade 1, 2, 3 | Grade 0 | | | | | | | | |
| Genotype | n = 45 | n = 99 | | | | | | | | |
| T/T or T/C | 18 | 21 | 0.02575 | 1.79 | 1.12 | 2.87 | 116 | 404 | 405 | 635 |
| C/C | 27 | 78 | | | | | | | | |
| rs4983545 | Grade 1, 2, 3 | Grade 0 | | | | | | | | |
| Genotype | n = 65 | n = 146 | | | | | | | | |
| C/C or C/T | 6 | 2 | 0.01151 | 2.58 | 1.64 | 4.06 | 136 | 444 | 445 | 655 |
| T/T | 59 | 144 | | | | | | | | |

TABLE 6
Breast cancer, late stage of 6 months, dermatitis

| | | | | | | | G | H | I | J |
|---|---|---|---|---|---|---|---|---|---|---|
| | B | C | D | E | F | | SEQ. | SEQ. | SEQ. | SEQ. |
| | Grade 1, 2, 3 | Grade 0 | Fisher | Relative | [95% confidence | | ID. | ID. | ID. | ID. |
| A | n = 11 | n = 131 | (p value) | risk | interval] | | NO. | NO. | NO. | NO. |
| rs2304136 | | | | | | | | | | |
| Genotype | | | | | | | | | | |
| G/G | 2 | 3 | 0.04818 | 6.09 | 1.75 | 21.16 | 85 | 342 | 343 | 604 |
| G/A or A/A | 9 | 128 | | | | | | | | |
| AB183822 | | | | | | | | | | |
| Genotype | | | | | | | | | | |
| A/A or A/G | 2 | 0 | 0.00549 | — | — | — | 1 | 174 | 175 | 520 |
| G/G | 9 | 131 | | | | | | | | |
| rs2272981 | | | | | | | | | | |
| Genotype | | | | | | | | | | |
| C/C | 2 | 3 | 0.04818 | 6.09 | 1.75 | 21.16 | 74 | 320 | 321 | 593 |
| C/A or A/A | 9 | 128 | | | | | | | | |
| rs2071863 | | | | | | | | | | |
| Genotype | | | | | | | | | | |
| G/G | 5 | 21 | 0.02976 | 3.72 | 1.23 | 11.26 | 50 | 272 | 273 | 569 |
| G/A or A/A | 6 | 110 | | | | | | | | |
| rs2304669 | | | | | | | | | | |
| Genotype | | | | | | | | | | |
| C/C or C/T | 6 | 31 | 0.03534 | 3.41 | 1.10 | 10.50 | 88 | 348 | 349 | 607 |
| T/T | 5 | 100 | | | | | | | | |
| rs1673041 | | | | | | | | | | |
| Genotype | | | | | | | | | | |
| T/T | 10 | 61 | 0.00887 | 10.00 | 1.31 | 76.08 | 30 | 232 | 233 | 549 |
| T/G or G/G | 1 | 70 | | | | | | | | |
| rs2288881 | | | | | | | | | | |
| Genotype | | | | | | | | | | |
| A/A | 2 | 3 | 0.04818 | 6.09 | 1.75 | 21.16 | 82 | 336 | 337 | 601 |
| A/G or G/G | 9 | 128 | | | | | | | | |
| rs972800 | | | | | | | | | | |
| Genotype | | | | | | | | | | |
| G/G | 3 | 5 | 0.01584 | 6.28 | 2.05 | 19.23 | 173 | 518 | 519 | 692 |
| G/T or T/T | 8 | 126 | | | | | | | | |
| rs2268332 | | | | | | | | | | |
| Genotype | | | | | | | | | | |
| G/G | 4 | 16 | 0.04968 | 3.49 | 1.12 | 10.83 | 67 | 306 | 307 | 586 |
| G/T or T/T | 7 | 115 | | | | | | | | |

TABLE 7
Breast cancer, late stage of 6 months, dermatitis

| | | | | | | | G | H | I | J |
|---|---|---|---|---|---|---|---|---|---|---|
| | | | D | E | F | | SEQ. | SEQ. | SEQ. | SEQ. |
| | | | Fisher | Relative | [95% confidence | | ID. | ID. | ID. | ID. |
| A | B | C | (p value) | risk | interval] | | NO. | NO. | NO. | NO. |
| rs2072817 | Grade 1, 2, 3 | Grade 0 | | | | | | | | |
| Genotype | n = 11 | n = 131 | | | | | | | | |
| C/C | 3 | 2 | 0.00323 | 10.28 | 3.85 | 27.43 | 51 | 274 | 275 | 570 |
| C/T or T/T | 8 | 129 | | | | | | | | |
| rs263438 | Grade 1, 2, 3 | Grade 0 | | | | | | | | |
| Genotype | n = 11 | n = 130 | | | | | | | | |
| C/C or C/A | 7 | 38 | 0.03728 | 3.73 | 1.15 | 12.11 | 97 | 366 | 367 | 616 |
| A/A | 4 | 92 | | | | | | | | |
| rs934945 | Grade 1, 2, 3 | Grade 0 | | | | | | | | |
| Genotype | n = 11 | n = 131 | | | | | | | | |
| C/C | 9 | 53 | 0.0105 | 5.81 | 1.30 | 25.92 | 172 | 516 | 517 | 691 |
| C/T or T/T | 2 | 78 | | | | | | | | |
| rs102275 | Grade 1, 2, 3 | Grade 0 | | | | | | | | |
| Genotype | n = 11 | n = 129 | | | | | | | | |
| C/C | 4 | 15 | 0.04355 | 3.64 | 1.18 | 11.26 | 180 | 181 | 523 | |
| C/T or T/T | 7 | 114 | | | | | | | | |
| rs2073747 | Grade 1, 2, 3 | Grade 0 | | | | | | | | |
| Genotype | n = 14 | n = 195 | | | | | | | | |
| G/G | 10 | 81 | 0.0476 | 3.24 | 1.05 | 10.00 | 54 | 280 | 281 | 573 |
| G/A or A/A | 4 | 114 | | | | | | | | |
| rs2238780 | Grade 1, 2, 3 | Grade 0 | | | | | | | | |
| Genotype | n = 14 | n = 197 | | | | | | | | |
| G/G | 10 | 77 | 0.02373 | 3.56 | 1.15 | 10.99 | 60 | 292 | 293 | 579 |
| G/C or C/C | 4 | 120 | | | | | | | | |
| rs2240718 | Grade 1, 2, 3 | Grade 0 | | | | | | | | |
| Genotype | n = 14 | n = 197 | | | | | | | | |
| G/G | 10 | 79 | 0.02657 | 3.43 | 1.11 | 10.58 | 62 | 296 | 297 | 581 |
| G/T or T/T | 4 | 118 | | | | | | | | |
| rs2241502 | Grade 1, 2, 3 | Grade 0 | | | | | | | | |
| Genotype | n = 40 | n = 41 | | | | | | | | |
| G/G | 21 | 10 | 0.0122 | 1.78 | 1.16 | 2.74 | 63 | 298 | 299 | 582 |
| G/A or A/A | 19 | 31 | | | | | | | | |

TABLE 8
Cervical cancer, early stage, intestinal disorder

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs1971783 | Grade 1, 2, 3 | Grade 0 | | | | | | | | |
| Genotype | n = 27 | n = 15 | | | | | | | | |
| T/T | 12 | 2 | 0.04932 | 1.60 | 1.07 | 2.40 | 44 | 260 | 261 | 563 |
| T/C or C/C | 15 | 13 | | | | | | | | |
| rs2072817 | Grade 1, 2, 3 | Grade 0 | | | | | | | | |
| Genotype | n = 27 | n = 15 | | | | | | | | |
| T/T | 24 | 7 | 0.00787 | 2.84 | 1.06 | 7.59 | 52 | 276 | 277 | 571 |
| T/C or C/C | 3 | 8 | | | | | | | | |
| rs2276015 | Grade 1, 2, 3 | Grade 0 | | | | | | | | |
| Genotype | n = 27 | n = 15 | | | | | | | | |
| A/A or A/G | 12 | 1 | 0.01481 | 1.78 | 1.21 | 2.62 | 76 | 324 | 325 | 595 |
| G/G | 15 | 14 | | | | | | | | |
| rs2305540 | Grade 1, 2, 3 | Grade 0 | | | | | | | | |
| Genotype | n = 27 | n = 15 | | | | | | | | |
| G/G | 14 | 2 | 0.02034 | 1.75 | 1.14 | 2.68 | 89 | 350 | 351 | 608 |
| G/A or A/A | 13 | 13 | | | | | | | | |
| rs3741049 | Grade 2, 3 | Grade 0, 1 | | | | | | | | |
| Genotype | n = 13 | n = 28 | | | | | | | | |
| G/G | 10 | 8 | 0.00633 | 4.26 | 1.37 | 13.23 | 114 | 400 | 401 | 633 |
| G/A or A/A | 3 | 20 | | | | | | | | |
| rs2075784 | Grade 1, 2, 3 | Grade 0 | | | | | | | | |
| Genotype | n = 27 | n = 13 | | | | | | | | |
| A/A or A/G | 19 | 4 | 0.03831 | 1.76 | 1.03 | 3.01 | 56 | 284 | 285 | 575 |
| G/G | 8 | 9 | | | | | | | | |
| rs2267437 | Grade 1, 2, 3 | Grade 0 | | | | | | | | |
| Genotype | n = 27 | n = 15 | | | | | | | | |
| C/C | 13 | 2 | 0.0423 | 1.67 | 1.10 | 2.53 | 65 | 302 | 303 | 584 |
| C/G or G/G | 14 | 13 | | | | | | | | |
| rs1968415 | Grade 1, 2, 3 | Grade 0 | | | | | | | | |
| Genotype | n = 27 | n = 15 | | | | | | | | |
| T/T or T/C | 16 | 3 | 0.02318 | 1.76 | 1.10 | 2.81 | 43 | 258 | 259 | 562 |
| C/C | 11 | 12 | | | | | | | | |
| rs1405655 | Grade 1, 2, 3 | Grade 0 | | | | | | | | |
| Genotype | n = 27 | n = 15 | | | | | | | | |
| T/T or T/C | 27 | 10 | 0.00353 | — | — | — | 23 | 218 | 219 | 542 |
| C/C | 0 | 5 | | | | | | | | |
| rs2445837 | Grade 1, 2, 3 | Grade 0 | | | | | | | | |
| Genotype | n = 27 | n = 15 | | | | | | | | |
| T/T or T/C | 27 | 10 | 0.00353 | | | | 93 | 358 | 359 | 612 |
| C/C | 0 | 5 | | | | | | | | |
| rs1351978 | Grade 1, 2, 3 | Grade 0 | | | | | | | | |
| Genotype | n = 27 | n = 15 | | | | | | | | |
| A/A | 24 | 7 | 0.00787 | 2.84 | 1.06 | 7.59 | 22 | 216 | 217 | 541 |
| A/C or C/C | 3 | 8 | | | | | | | | |

TABLE 9
Cervical cancer, early stage, intestinal disorder

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs2248574 | Grade 1, 2, 3 | Grade 0 | | | | | | | | |
| Genotype | n = 27 | n = 15 | | | | | | | | |
| T/T or T/C | 27 | 11 | 0.0122 | — | — | — | 64 | 300 | 301 | 583 |
| C/C | 0 | 4 | | | | | | | | |
| rs1684385 | Grade 1, 2, 3 | Grade 0 | | | | | | | | |
| Genotype | n = 27 | n = 15 | | | | | | | | |
| A/A | 22 | 6 | 0.01479 | 2.20 | 1.06 | 4.56 | 31 | 234 | 235 | 550 |
| A/G or G/G | 5 | 9 | | | | | | | | |
| rs458486 | Grade 1, 2, 3 | Grade 0 | | | | | | | | |
| Genotype | n = 27 | n = 15 | | | | | | | | |
| T/T or T/C | 26 | 10 | 0.01641 | 4.33 | 0.72 | 26.23 | 129 | 430 | 431 | 648 |
| C/C | 1 | 5 | | | | | | | | |
| rs2304579 | Grade 1, 2, 3 | Grade 0 | | | | | | | | |
| Genotype | n = 27 | n = 15 | | | | | | | | |
| A/A | 27 | 12 | 0.03963 | — | — | — | 86 | 344 | 345 | 605 |
| A/G or G/G | 0 | 3 | | | | | | | | |
| rs165815 | Grade 1, 2, 3 | Grade 0 | | | | | | | | |
| Genotype | n = 34 | n = 20 | | | | | | | | |
| C/C | 16 | 3 | 0.02067 | 1.64 | 1.12 | 2.39 | 29 | 230 | 231 | 548 |
| C/T or T/T | 18 | 17 | | | | | | | | |
| rs2238780 | Grade 1, 2, 3 | Grade 0 | | | | | | | | |
| Genotype | n = 34 | n = 20 | | | | | | | | |
| G/G | 16 | 3 | 0.02067 | 1.64 | 1.12 | 2.39 | 60 | 292 | 293 | 579 |
| G/C or C/C | 18 | 17 | | | | | | | | |
| rs1805312 | Grade 2, 3 | Grade 0, 1 | | | | | | | | |
| Genotype | n = 13 | n = 29 | | | | | | | | |
| G/G or G/C | 8 | 8 | 0.04721 | 2.60 | 1.03 | 6.57 | 34 | 240 | 241 | 553 |
| C/C | 5 | 21 | | | | | | | | |
| rs491071 | Grade 2, 3 | Grade 0, 1 | | | | | | | | |
| Genotype | n = 13 | n = 29 | | | | | | | | |
| C/C | 12 | 13 | 0.00557 | 8.16 | 1.17 | 57.05 | 134 | 440 | 441 | 653 |
| C/G or G/G | 1 | 16 | | | | | | | | |
| rs73234 | Grade 2, 3 | Grade 0, 1 | | | | | | | | |
| Genotype | n = 13 | n = 29 | | | | | | | | |
| G/G or G/C | 9 | 10 | 0.04937 | 2.72 | 0.99 | 7.47 | 157 | 486 | 487 | 676 |
| C/C | 4 | 19 | | | | | | | | |
| rs9226 | Grade 2, 3 | Grade 0, 1 | | | | | | | | |
| Genotype | n = 13 | n = 29 | | | | | | | | |
| T/T | 3 | 0 | 0.02491 | — | — | — | 171 | 514 | 515 | 690 |
| T/A or A/A | 10 | 29 | | | | | | | | |

TABLE 10
Cervical cancer, early stage, intestinal disorder

| | | | | | F | | G | H | I | J |
|---|---|---|---|---|---|---|---|---|---|---|
| | B | C | D | E | [95% | | SEQ. | SEQ. | SEQ. | SEQ. |
| | Grade 2, 3 | Grade 0, 1 | Fisher | Relative | confidence | | ID. | ID. | ID. | ID. |
| A | n = 13 | n = 29 | (p value) | risk | interval] | | NO. | NO. | NO. | NO. |
| rs227053 | | | | | | | | | | |
| Genotype | | | | | | | | | | |
| T/T | 8 | 4 | 0.00301 | 4.00 | 1.64 | 9.79 | 70 | 312 | 313 | 589 |
| T/A or A/A | 5 | 25 | | | | | | | | |
| rs664677 | | | | | | | | | | |
| Genotype | | | | | | | | | | |
| T/T | 7 | 3 | 0.00463 | 3.73 | 1.63 | 8.54 | 153 | 478 | 479 | 672 |
| T/C or C/C | 6 | 26 | | | | | | | | |
| rs645485 | | | | | | | | | | |
| Genotype | | | | | | | | | | |
| G/G | 7 | 3 | 0.00463 | 3.73 | 1.63 | 8.54 | 150 | 472 | 473 | 669 |
| G/A or A/A | 6 | 26 | | | | | | | | |
| rs664982 | | | | | | | | | | |
| Genotype | | | | | | | | | | |
| T/T | 8 | 5 | 0.00936 | 3.57 | 1.44 | 8.83 | 154 | 480 | 481 | 673 |
| T/C or C/C | 5 | 24 | | | | | | | | |
| rs652541 | | | | | | | | | | |
| Genotype | | | | | | | | | | |
| G/G | 8 | 5 | 0.00936 | 3.57 | 1.44 | 8.83 | 152 | 476 | 477 | 671 |
| G/A or A/A | 5 | 24 | | | | | | | | |
| rs641605 | | | | | | | | | | |
| Genotype | | | | | | | | | | |
| T/T | 8 | 5 | 0.00936 | 3.57 | 1.44 | 8.83 | 149 | 470 | 471 | 668 |
| T/C or C/C | 5 | 24 | | | | | | | | |
| rs625120 | | | | | | | | | | |
| Genotype | | | | | | | | | | |
| G/G | 8 | 5 | 0.00936 | 3.57 | 1.44 | 8.83 | 146 | 464 | 465 | 665 |
| G/A or A/A | 5 | 24 | | | | | | | | |
| rs227077 | | | | | | | | | | |
| Genotype | | | | | | | | | | |
| T/T | 8 | 5 | 0.00936 | 3.57 | 1.44 | 8.83 | 72 | 316 | 317 | 591 |
| T/C or C/C | 5 | 24 | | | | | | | | |
| rs228589 | | | | | | | | | | |
| Genotype | | | | | | | | | | |
| T/T | 8 | 8 | 0.04721 | 2.60 | 1.03 | 6.57 | 80 | 332 | 333 | 599 |
| T/A or A/A | 5 | 21 | | | | | | | | |
| rs609557 | | | | | | | | | | |
| Genotype | | | | | | | | | | |
| T/T | 8 | 8 | 0.04721 | 2.60 | 1.03 | 6.57 | 144 | 460 | 461 | 663 |
| T/G or G/G | 5 | 21 | | | | | | | | |

TABLE 11
Cervical cancer, early stage, intestinal disorder

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs183460 | Grade 2, 3 | Grade 0, 1 | | | | | | | | |
| Genotype | n = 13 | n = 29 | | | | | | | | |
| C/C | 8 | 8 | 0.04721 | 2.60 | 1.03 | 6.57 | 36 | 244 | 245 | 555 |
| C/A or A/A | 5 | 21 | | | | | | | | |
| rs2274760 | Grade 2, 3 | Grade 0, 1 | | | | | | | | |
| Genotype | n = 13 | n = 29 | | | | | | | | |
| C/C or C/G | 3 | 0 | 0.02491 | — | — | — | 75 | 322 | 323 | 594 |
| G/G | 10 | 29 | | | | | | | | |
| rs3781868 | Grade 2, 3 | Grade 0, 1 | | | | | | | | |
| Genotype | n = 13 | n = 29 | | | | | | | | |
| G/G | 8 | 8 | 0.04721 | 2.60 | 1.03 | 6.57 | 118 | 408 | 409 | 637 |
| G/T or T/T | 5 | 21 | | | | | | | | |
| rs2705 | Grade 2, 3 | Grade 0, 1 | | | | | | | | |
| Genotype | n = 13 | n = 29 | | | | | | | | |
| T/T or T/C | 9 | 10 | 0.04937 | 2.72 | 0.99 | 7.47 | 98 | 368 | 369 | 617 |
| C/C | 4 | 19 | | | | | | | | |
| rs243387 | Grade 2, 3 | Grade 0, 1 | | | | | | | | |
| Genotype | n = 13 | n = 29 | | | | | | | | |
| T/T or T/C | 9 | 10 | 0.04937 | 2.72 | 0.99 | 7.47 | 91 | 354 | 355 | 610 |
| C/C | 4 | 19 | | | | | | | | |
| rs1002481 | Grade 2, 3 | Grade 0, 1 | | | | | | | | |
| Genotype | n = 13 | n = 29 | | | | | | | | |
| T/T or T/A | 6 | 4 | 0.04594 | 2.74 | 1.20 | 6.27 | 2 | 176 | 177 | 521 |
| A/A | 7 | 25 | | | | | | | | |
| rs2854461 | Grade 2, 3 | Grade 0, 1 | | | | | | | | |
| Genotype | n = 13 | n = 29 | | | | | | | | |
| A/A | 7 | 5 | 0.02613 | 2.92 | 1.23 | 6.90 | 105 | 382 | 383 | 624 |
| A/C or C/C | 6 | 24 | | | | | | | | |
| rs3803798 | Grade 2, 3 | Grade 0, 1 | | | | | | | | |
| Genotype | n = 13 | n = 29 | | | | | | | | |
| G/G | 6 | 3 | 0.01571 | 3.14 | 1.41 | 7.02 | 121 | 414 | 415 | 640 |
| G/C or C/C | 7 | 26 | | | | | | | | |
| rs573890 | Grade 2, 3 | Grade 0, 1 | | | | | | | | |
| Genotype | n = 13 | n = 29 | | | | | | | | |
| G/G | 8 | 5 | 0.00936 | 3.57 | 1.44 | 8.83 | 141 | 454 | 455 | 660 |
| G/C or C/C | 5 | 24 | | | | | | | | |
| rs227055 | Grade 2, 3 | Grade 0, 1 | | | | | | | | |
| Genotype | n = 13 | n = 28 | | | | | | | | |
| A/A | 8 | 5 | 0.01019 | 3.45 | 1.40 | 8.50 | 71 | 314 | 315 | 590 |
| A/G or G/G | 5 | 23 | | | | | | | | |

TABLE 12
Cervical cancer, late stage of 6 months, intestinal disorder

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs1845452 | Grade 1, 2, 3 | Grade 0 | | | | | | | | |
| Genotype | n = 7 | n = 34 | | | | | | | | |
| C/C | 2 | 0 | 0.02561 | — | — | — | 37 | 246 | 247 | 556 |
| C/G or G/G | 5 | 34 | | | | | | | | |
| rs704227 | Grade 1, 2, 3 | Grade 0 | | | | | | | | |
| Genotype | n = 7 | n = 34 | | | | | | | | |
| T/T | 4 | 6 | 0.04749 | 4.13 | 1.11 | 15.42 | 155 | 482 | 483 | 674 |
| T/C or C/C | 3 | 28 | | | | | | | | |
| rs1047347 | Grade 1, 2, 3 | Grade 0 | | | | | | | | |
| Genotype | n = 7 | n = 34 | | | | | | | | |
| C/C | 5 | 9 | 0.03502 | 4.82 | 1.07 | 21.77 | 6 | 184 | 185 | 525 |
| C/A or A/A | 2 | 25 | | | | | | | | |
| rs752593 | Grade 1, 2, 3 | Grade 0 | | | | | | | | |
| Genotype | n = 7 | n = 34 | | | | | | | | |
| A/A or A/G | 7 | 17 | 0.02947 | — | — | — | 161 | 494 | 495 | 680 |
| G/G | 0 | 17 | | | | | | | | |
| rs518116 | Grade 1, 2, 3 | Grade 0 | | | | | | | | |
| Genotype | n = 7 | n = 34 | | | | | | | | |
| G/G | 7 | 16 | 0.01232 | — | — | — | 139 | 450 | 451 | 658 |
| G/A or A/A | 0 | 18 | | | | | | | | |
| rs1862392 | Grade 1, 2, 3 | Grade 0 | | | | | | | | |
| Genotype | n = 7 | n = 33 | | | | | | | | |
| A/A or A/T | 5 | 9 | 0.03927 | 4.64 | 1.03 | 20.93 | 39 | 250 | 251 | 558 |
| T/T | 2 | 24 | | | | | | | | |
| rs3136820 | Grade 1, 2, 3 | Grade 0 | | | | | | | | |
| Genotype | n = 7 | n = 34 | | | | | | | | |
| G/G | 2 | 0 | 0.02561 | — | — | — | 110 | 392 | 393 | 629 |
| G/T or T/T | 5 | 34 | | | | | | | | |
| rs2290679 | Grade 1, 2, 3 | Grade 0 | | | | | | | | |
| Genotype | n = 7 | n = 33 | | | | | | | | |
| T/T or T/C | 3 | 2 | 0.02987 | 5.25 | 1.63 | 16.87 | 83 | 338 | 339 | 602 |
| C/C | 4 | 31 | | | | | | | | |
| rs2270390 | Grade 1, 2, 3 | Grade 0 | | | | | | | | |
| Genotype | n = 7 | n = 34 | | | | | | | | |
| C/C | 4 | 6 | 0.04749 | 4.13 | 1.11 | 15.42 | 68 | 308 | 309 | 587 |
| C/T or T/T | 3 | 28 | | | | | | | | |
| rs2008521 | Grade 1, 2, 3 | Grade 0 | | | | | | | | |
| Genotype | n = 7 | n = 34 | | | | | | | | |
| T/T or T/C | 7 | 14 | 0.00862 | — | — | — | 46 | 246 | 265 | 565 |
| C/C | 0 | 20 | | | | | | | | |
| rs3786738 | Grade 1, 2, 3 | Grade 0 | | | | | | | | |
| Genotype | n = 7 | n = 34 | | | | | | | | |
| T/T or T/C | 7 | 14 | 0.00862 | — | — | — | 119 | 410 | 411 | 638 |
| C/C | 0 | 20 | | | | | | | | |
| rs1928156 | Grade 1, 2, 3 | Grade 0 | | | | | | | | |
| Genotype | n = 6 | n = 34 | | | | | | | | |
| A/A | 2 | 0 | 0.01923 | — | — | — | 42 | 256 | 257 | 561 |
| A/G or G/G | 4 | 34 | | | | | | | | |
| rs5745095 | Grade 1, 2, 3 | Grade 0 | | | | | | | | |
| Genotype | n = 6 | n = 34 | | | | | | | | |
| T/T or T/C | 2 | 0 | 0.01923 | — | — | — | 142 | 456 | 457 | 661 |
| C/C | 4 | 34 | | | | | | | | |
| rs190246 | Grade 1, 2, 3 | Grade 0 | | | | | | | | |
| Genotype | n = 7 | n = 34 | | | | | | | | |
| T/T or T/G | 7 | 19 | 0.03533 | — | — | — | 41 | 254 | 255 | 560 |
| G/G | 0 | 15 | | | | | | | | |

TABLE 13
Prostate cancer, early stage, ureteropathy

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs157703 | Grade 1, 2, 3 | Grade 0 | | | | | | | | |
| Genotype | n = 32 | n = 36 | | | | | | | | |
| T/T | 11 | 4 | 0.03816 | 1.85 | 1.18 | 2.91 | 28 | 228 | 229 | 547 |
| T/C or C/C | 21 | 32 | | | | | | | | |
| rs1475489 | Grade 1, 2, 3 | Grade 0 | | | | | | | | |
| Genotype | n = 31 | n = 38 | | | | | | | | |
| T/T | 6 | 1 | 0.04007 | 2.13 | 1.39 | 3.26 | 24 | 220 | 221 | 543 |
| T/A or A/A | 25 | 37 | | | | | | | | |
| rs2267437 | Grade 1, 2, 3 | Grade 0 | | | | | | | | |
| Genotype | n = 32 | n = 38 | | | | | | | | |
| G/G or G/C | 25 | 20 | 0.04425 | 1.98 | 1.00 | 3.92 | 66 | 304 | 305 | 585 |
| C/C | 7 | 18 | | | | | | | | |
| rs3809454 | Grade 1, 2, 3 | Grade 0 | | | | | | | | |
| Genotype | n = 32 | n = 38 | | | | | | | | |
| C/C | 11 | 3 | 0.00747 | 2.10 | 1.36 | 3.24 | 126 | 424 | 425 | 645 |
| C/A or A/A | 21 | 35 | | | | | | | | |
| rs2705 | Grade 1, 2, 3 | Grade 0 | | | | | | | | |
| Genotype | n = 32 | n = 38 | | | | | | | | |
| C/C or C/T | 32 | 31 | 0.01334 | — | — | — | 99 | 370 | 371 | 618 |
| T/T | 0 | 7 | | | | | | | | |
| rs243387 | Grade 1, 2, 3 | Grade 0 | | | | | | | | |
| Genotype | n = 32 | n = 38 | | | | | | | | |
| C/C or C/T | 32 | 31 | 0.01334 | — | — | — | 92 | 356 | 357 | 611 |
| T/T | 0 | 7 | | | | | | | | |
| rs3791213 | Grade 1, 2, 3 | Grade 0 | | | | | | | | |
| Genotype | n = 32 | n = 38 | | | | | | | | |
| T/T | 24 | 18 | 0.0274 | 2.00 | 1.05 | 3.80 | 120 | 412 | 413 | 639 |
| T/C or C/C | 8 | 20 | | | | | | | | |
| rs1059234 | Grade 1, 2, 3 | Grade 0 | | | | | | | | |
| Genotype | n = 32 | n = 37 | | | | | | | | |
| C/C or C/T | 29 | 26 | 0.04169 | 2.46 | 0.88 | 6.92 | 8 | 188 | 189 | 527 |
| T/T | 3 | 11 | | | | | | | | |
| rs651646 | Grade 1, 2, 3 | Grade 0 | | | | | | | | |
| Genotype | n = 31 | n = 38 | | | | | | | | |
| A/A or A/T | 22 | 15 | 0.01478 | 2.11 | 1.14 | 3.91 | 151 | 474 | 475 | 670 |
| T/T | 9 | 23 | | | | | | | | |
| rs875382 | Grade 1, 2, 3 | Grade 0 | | | | | | | | |
| Genotype | n = 32 | n = 38 | | | | | | | | |
| C/C or C/T | 31 | 30 | 0.03315 | 4.57 | 0.71 | 29.51 | 169 | 510 | 511 | 688 |
| T/T | 1 | 8 | | | | | | | | |

TABLE 14
Prostate cancer, early stage, ureteropathy

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs2066505 | Grade 1, 2, 3 | Grade 0 | | | | | | | | |
| Genotype | n = 32 | n = 38 | | | | | | | | |
| A/A or A/G | 14 | 7 | 0.03518 | 1.81 | 1.13 | 2.92 | 48 | 268 | 269 | 567 |
| G/G | 18 | 31 | | | | | | | | |
| rs1045376 | Grade 1, 2, 3 | Grade 0 | | | | | | | | |
| Genotype | n = 32 | n = 38 | | | | | | | | |
| T/T or T/C | 14 | 6 | 0.01603 | 1.94 | 1.22 | 3.10 | 5 | 182 | 183 | 524 |
| C/C | 18 | 32 | | | | | | | | |
| rs282065 | Grade 1, 2, 3 | Grade 0 | | | | | | | | |
| Genotype | n = 32 | n = 38 | | | | | | | | |
| A/A | 11 | 5 | 0.04713 | 1.77 | 1.10 | 2.83 | 103 | 378 | 379 | 622 |
| A/G or G/G | 21 | 33 | | | | | | | | |
| rs2750440 | Grade 1, 2, 3 | Grade 0 | | | | | | | | |
| Genotype | n = 32 | n = 38 | | | | | | | | |
| T/T or T/C | 12 | 5 | 0.02536 | 1.87 | 1.18 | 2.97 | 102 | 376 | 377 | 621 |
| C/C | 20 | 33 | | | | | | | | |
| rs2616023 | Grade 1, 2, 3 | Grade 0 | | | | | | | | |
| Genotype | n = 32 | n = 38 | | | | | | | | |
| G/G or G/A | 12 | 5 | 0.02536 | 1.87 | 1.18 | 2.97 | 96 | 364 | 365 | 615 |
| A/A | 20 | 33 | | | | | | | | |
| rs2287830 | Grade 1, 2, 3 | Grade 0 | | | | | | | | |
| Genotype | n = 32 | n = 38 | | | | | | | | |
| T/T or T/G | 4 | 0 | 0.03922 | — | — | — | 81 | 334 | 335 | 600 |
| G/G | 28 | 38 | | | | | | | | |
| rs73234 | Grade 1, 2, 3 | Grade 0 | | | | | | | | |
| Genotype | n = 32 | n = 38 | | | | | | | | |
| C/C or C/G | 32 | 31 | 0.01334 | — | — | — | 158 | 488 | 489 | 677 |
| G/G | 0 | 7 | | | | | | | | |
| rs1801270 | Grade 1, 2, 3 | Grade 0 | | | | | | | | |
| Genotype | n = 31 | n = 38 | | | | | | | | |
| C/C or C/A | 29 | 27 | 0.02815 | 3.37 | 0.92 | 12.35 | 33 | 238 | 239 | 552 |
| A/A | 2 | 11 | | | | | | | | |
| rs1520483 | Grade 1, 2, 3 | Grade 0 | | | | | | | | |
| Genotype | n = 32 | n = 38 | | | | | | | | |
| T/T or T/C | 27 | 23 | 0.03528 | 2.16 | 0.97 | 4.81 | 25 | 222 | 223 | 544 |
| C/C | 5 | 15 | | | | | | | | |
| rs871027 | Grade 1, 2, 3 | Grade 0 | | | | | | | | |
| Genotype | n = 32 | n = 38 | | | | | | | | |
| A/A | 4 | 0 | 0.03922 | — | — | — | 168 | 508 | 509 | 687 |
| A/G or G/G | 28 | 38 | | | | | | | | |

TABLE 15
Prostate cancer, late stage of 3 months, ureteropathy

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs3131687 | Grade 1, 2, 3 | Grade 0 | | | | | | | | |
| Genotype | n = 37 | n = 33 | | | | | | | | |
| A/A or A/G | 33 | 21 | 0.02075 | 2.44 | 1.02 | 5.86 | 109 | 390 | 391 | 628 |
| G/G | 4 | 12 | | | | | | | | |
| rs4543783 | Grade 1, 2, 3 | Grade 0 | | | | | | | | |
| Genotype | n = 37 | n = 33 | | | | | | | | |
| G/G or G/C | 33 | 21 | 0.02075 | 2.44 | 1.02 | 5.86 | 128 | 428 | 429 | 647 |
| C/C | 4 | 12 | | | | | | | | |
| rs3806201 | Grade 1, 2, 3 | Grade 0 | | | | | | | | |
| Genotype | n = 37 | n = 33 | | | | | | | | |
| A/A or A/C | 37 | 29 | 0.04463 | — | — | — | 122 | 416 | 417 | 641 |
| C/C | 0 | 4 | | | | | | | | |
| rs3806202 | Grade 1, 2, 3 | Grade 0 | | | | | | | | |
| Genotype | n = 37 | n = 33 | | | | | | | | |
| C/C or C/T | 37 | 29 | 0.04463 | — | — | — | 123 | 418 | 419 | 642 |
| T/T | 0 | 4 | | | | | | | | |
| rs1059234 | Grade 1, 2, 3 | Grade 0 | | | | | | | | |
| Genotype | n = 36 | n = 33 | | | | | | | | |
| C/C or C/T | 34 | 21 | 0.0021 | 4.33 | 1.18 | 15.88 | 8 | 188 | 189 | 527 |
| T/T | 2 | 12 | | | | | | | | |
| rs1801270 | Grade 1, 2, 3 | Grade 0 | | | | | | | | |
| Genotype | n = 36 | n = 33 | | | | | | | | |
| C/C or C/A | 35 | 21 | 0.00041 | 8.13 | 1.22 | 54.00 | 33 | 238 | 239 | 552 |
| A/A | 1 | 12 | | | | | | | | |
| rs2854455 | Grade 1, 2, 3 | Grade 0 | | | | | | | | |
| Genotype | n = 37 | n = 33 | | | | | | | | |
| T/T | 35 | 24 | 0.01925 | 3.26 | 0.92 | 11.63 | 104 | 380 | 381 | 623 |
| T/C or C/C | 2 | 9 | | | | | | | | |
| rs704227 | Grade 1, 2, 3 | Grade 0 | | | | | | | | |
| Genotype | n = 37 | n = 33 | | | | | | | | |
| C/C or C/T | 30 | 19 | 0.03956 | 1.84 | 0.96 | 3.50 | 156 | 484 | 485 | 675 |
| T/T | 7 | 14 | | | | | | | | |
| rs1171097 | Grade 1, 2, 3 | Grade 0 | | | | | | | | |
| Genotype | n = 37 | n = 33 | | | | | | | | |
| G/G or G/C | 20 | 8 | 0.01488 | 1.76 | 1.14 | 2.73 | 18 | 208 | 209 | 537 |
| C/C | 17 | 25 | | | | | | | | |
| rs274867 | Grade 1, 2, 3 | Grade 0 | | | | | | | | |
| Genotype | n = 37 | n = 33 | | | | | | | | |
| A/A or A/G | 37 | 29 | 0.04463 | — | — | — | 101 | 374 | 375 | 620 |
| G/G | 0 | 4 | | | | | | | | |
| rs2007182 | Grade 1, 2, 3 | Grade 0 | | | | | | | | |
| Genotype | n = 37 | n = 33 | | | | | | | | |
| G/G | 36 | 27 | 0.04634 | 4.00 | 0.64 | 24.86 | 45 | 262 | 263 | 564 |
| G/A or A/A | 1 | 6 | | | | | | | | |
| rs2066505 | Grade 1, 2, 3 | Grade 0 | | | | | | | | |
| Genotype | n = 37 | n = 33 | | | | | | | | |
| A/A or A/G | 16 | 5 | 0.01769 | 1.78 | 1.19 | 2.66 | 48 | 268 | 269 | 567 |
| G/G | 21 | 28 | | | | | | | | |
| rs2304580 | Grade 1, 2, 3 | Grade 0 | | | | | | | | |
| Genotype | n = 37 | n = 33 | | | | | | | | |
| T/T | 6 | 0 | 0.02618 | — | — | — | 87 | 346 | 347 | 606 |
| T/C or C/C | 31 | 33 | | | | | | | | |
| rs3733995 | Grade 1, 2, 3 | Grade 0 | | | | | | | | |
| Genotype | n = 37 | n = 33 | | | | | | | | |
| T/T or T/C | 9 | 2 | 0.04961 | 1.72 | 1.17 | 2.54 | 111 | 394 | 395 | 630 |
| C/C | 28 | 31 | | | | | | | | |

TABLE 16
Prostate cancer, late stage of 3 months, ureteropathy

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs3806207 | Grade 1, 2, 3 | Grade 0 | | | | | | | | |
| Genotype | n = 37 | n = 33 | | | | | | | | |
| T/T or T/G | 37 | 29 | 0.04463 | — | — | — | 125 | 422 | 423 | 644 |
| G/G | 0 | 4 | | | | | | | | |
| rs3806204 | Grade 1, 2, 3 | Grade 0 | | | | | | | | |
| Genotype | n = 37 | n = 33 | | | | | | | | |
| G/G or G/A | 37 | 29 | 0.04463 | — | — | — | 124 | 420 | 421 | 643 |
| A/A | 0 | 4 | | | | | | | | |
| rs1126973 | Grade 1, 2, 3 | Grade 0 | | | | | | | | |
| Genotype | n = 37 | n = 33 | | | | | | | | |
| T/T or T/C | 36 | 27 | 0.04634 | 4.00 | 0.64 | 24.86 | 12 | 196 | 197 | 531 |
| C/C | 1 | 6 | | | | | | | | |
| rs1126970 | Grade 1, 2, 3 | Grade 0 | | | | | | | | |
| Genotype | n = 37 | n = 33 | | | | | | | | |
| C/C or C/A | 36 | 27 | 0.04634 | 4.00 | 0.64 | 24.86 | 10 | 192 | 193 | 529 |
| A/A | 1 | 6 | | | | | | | | |
| rs1126972 | Grade 1, 2, 3 | Grade 0 | | | | | | | | |
| Genotype | n = 37 | n = 33 | | | | | | | | |
| C/C or C/G | 36 | 27 | 0.04634 | 4.00 | 0.64 | 24.86 | 11 | 194 | 195 | 530 |
| G/G | 1 | 6 | | | | | | | | |
| rs2270390 | Grade 1, 2, 3 | Grade 0 | | | | | | | | |
| Genotype | n = 37 | n = 33 | | | | | | | | |
| T/T or T/C | 30 | 19 | 0.03956 | 1.84 | 0.96 | 3.50 | 69 | 310 | 311 | 588 |
| C/C | 7 | 14 | | | | | | | | |
| rs2742946 | Grade 1, 2, 3 | Grade 0 | | | | | | | | |
| Genotype | n = 37 | n = 33 | | | | | | | | |
| T/T or T/C | 30 | 18 | 0.02164 | 1.96 | 1.03 | 3.76 | 100 | 372 | 373 | 619 |
| C/C | 7 | 15 | | | | | | | | |
| rs1045376 | Grade 1, 2, 3 | Grade 0 | | | | | | | | |
| Genotype | n = 37 | n = 33 | | | | | | | | |
| T/T or T/C | 15 | 5 | 0.03267 | 1.70 | 1.14 | 2.55 | 5 | 182 | 183 | 524 |
| C/C | 22 | 28 | | | | | | | | |
| rs13436 | Grade 1, 2, 3 | Grade 0 | | | | | | | | |
| Genotype | n = 37 | n = 33 | | | | | | | | |
| C/C or C/G | 37 | 29 | 0.04463 | — | — | — | 21 | 214 | 215 | 540 |
| G/G | 0 | 4 | | | | | | | | |
| rs750621 | Grade 1, 2, 3 | Grade 0 | | | | | | | | |
| Genotype | n = 37 | n = 33 | | | | | | | | |
| G/G | 36 | 27 | 0.04634 | 4.00 | 0.64 | 24.86 | 160 | 492 | 493 | 679 |
| G/T or T/T | 1 | 6 | | | | | | | | |
| rs3744357 | Grade 1, 2, 3 | Grade 0 | | | | | | | | |
| Genotype | n = 37 | n = 33 | | | | | | | | |
| T/T or T/C | 15 | 5 | 0.03267 | 1.70 | 1.14 | 2.55 | 116 | 404 | 405 | 635 |
| C/C | 22 | 28 | | | | | | | | |
| rs2075747 | Grade 1, 2, 3 | Grade 0 | | | | | | | | |
| Genotype | n = 37 | n = 33 | | | | | | | | |
| G/G or G/A | 37 | 28 | 0.01961 | — | — | — | 55 | 282 | 283 | 574 |
| A/A | 0 | 5 | | | | | | | | |
| rs3742557 | Grade 1, 2, 3 | Grade 0 | | | | | | | | |
| Genotype | n = 45 | n = 35 | | | | | | | | |
| A/A or A/G | 29 | 14 | 0.04198 | 1.56 | 1.02 | 2.38 | 115 | 402 | 403 | 634 |
| G/G | 16 | 21 | | | | | | | | |

TABLE 17
Prostate cancer, late stage of 6 months, ureteropathy

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs1045376 | Grade 1, 2, 3 | Grade 0 | | | | | | | | |
| Genotype | n = 33 | n = 37 | | | | | | | | |
| T/T or T/C | 15 | 5 | 0.00393 | 2.08 | 1.33 | 3.26 | 5 | 182 | 183 | 524 |
| C/C | 18 | 32 | | | | | | | | |
| rs2010352 | Grade 1, 2, 3 | Grade 0 | | | | | | | | |
| Genotype | n = 33 | n = 37 | | | | | | | | |
| G/G | 15 | 6 | 0.00976 | 1.94 | 1.23 | 3.07 | 47 | 266 | 267 | 566 |
| G/A or A/A | 18 | 31 | | | | | | | | |
| rs1862391 | Grade 1, 2, 3 | Grade 0 | | | | | | | | |
| Genotype | n = 33 | n = 37 | | | | | | | | |
| G/G or G/T | 18 | 9 | 0.01385 | 1.91 | 1.17 | 3.11 | 38 | 248 | 249 | 557 |
| T/T | 15 | 28 | | | | | | | | |
| rs2910199 | Grade 1, 2, 3 | Grade 0 | | | | | | | | |
| Genotype | n = 33 | n = 37 | | | | | | | | |
| A/A or A/G | 18 | 9 | 0.01385 | 1.91 | 1.17 | 3.11 | 107 | 386 | 387 | 626 |
| G/G | 15 | 28 | | | | | | | | |
| rs1862392 | Grade 1, 2, 3 | Grade 0 | | | | | | | | |
| Genotype | n = 33 | n = 37 | | | | | | | | |
| A/A or A/T | 18 | 9 | 0.01385 | 1.91 | 1.17 | 3.11 | 39 | 250 | 251 | 558 |
| T/T | 15 | 28 | | | | | | | | |
| rs1805414 | Grade 1, 2, 3 | Grade 0 | | | | | | | | |
| Genotype | n = 33 | n = 37 | | | | | | | | |
| G/G | 25 | 18 | 0.02732 | 1.96 | 1.04 | 3.70 | 35 | 242 | 243 | 554 |
| G/A or A/A | 8 | 19 | | | | | | | | |
| rs1104893 | Grade 1, 2, 3 | Grade 0 | | | | | | | | |
| Genotype | n = 33 | n = 37 | | | | | | | | |
| G/G | 25 | 18 | 0.02732 | 1.96 | 1.04 | 3.70 | 9 | 190 | 191 | 528 |
| G/A or A/A | 8 | 19 | | | | | | | | |
| rs2283264 | Grade 1, 2, 3 | Grade 0 | | | | | | | | |
| Genotype | n = 33 | n = 37 | | | | | | | | |
| C/C | 32 | 29 | 0.03 | 4.72 | 0.73 | 30.43 | 79 | 330 | 331 | 598 |
| C/G or G/G | 1 | 8 | | | | | | | | |
| rs791040 | Grade 1, 2, 3 | Grade 0 | | | | | | | | |
| Genotype | n = 33 | n = 37 | | | | | | | | |
| G/G | 13 | 6 | 0.03513 | 1.74 | 1.10 | 2.76 | 164 | 500 | 501 | 683 |
| G/A or A/A | 20 | 31 | | | | | | | | |
| rs791041 | Grade 1, 2, 3 | Grade 0 | | | | | | | | |
| Genotype | n = 33 | n = 37 | | | | | | | | |
| T/T | 13 | 6 | 0.03513 | 1.74 | 1.10 | 2.76 | 166 | 504 | 505 | 685 |
| T/A or A/A | 20 | 31 | | | | | | | | |
| rs1144153 | Grade 1, 2, 3 | Grade 0 | | | | | | | | |
| Genotype | n = 33 | n = 37 | | | | | | | | |
| T/T | 13 | 6 | 0.03513 | 1.74 | 1.10 | 2.76 | 14 | 200 | 201 | 533 |
| T/C or C/C | 20 | 31 | | | | | | | | |
| rs2190935 | Grade 1, 2, 3 | Grade 0 | | | | | | | | |
| Genotype | n = 32 | n = 37 | | | | | | | | |
| T/T | 4 | 0 | 0.0416 | — | — | — | 57 | 286 | 287 | 576 |
| T/C or C/C | 28 | 37 | | | | | | | | |
| rs2561829 | Grade 1, 2, 3 | Grade 0 | | | | | | | | |
| Genotype | n = 33 | n = 37 | | | | | | | | |
| T/T or T/C | 18 | 9 | 0.01385 | 1.91 | 1.17 | 3.11 | 95 | 362 | 363 | 614 |
| C/C | 15 | 28 | | | | | | | | |

TABLE 18
Prostate cancer, late stage of 6 months, ureteropathy

| | | | | | F | | G | H | I | J |
|---|---|---|---|---|---|---|---|---|---|---|
| | | | D | E | [95% | | SEQ. | SEQ. | SEQ. | SEQ. |
| | | | Fisher | Relative | confidence | | ID. | ID. | ID. | ID. |
| A | B | C | (p value) | risk | interval] | | NO. | NO. | NO. | NO. |
| rs4699052 | Grade 1, 2, 3 | Grade 0 | | | | | | | | |
| Genotype | n = 33 | n = 37 | | | | | | | | |
| C/C | 21 | 11 | 0.00779 | 2.08 | 1.22 | 3.53 | 130 | 432 | 433 | 649 |
| C/T or T/T | 12 | 26 | | | | | | | | |
| rs4699053 | Grade 1, 2, 3 | Grade 0 | | | | | | | | |
| Genotype | n = 33 | n = 37 | | | | | | | | |
| C/C | 23 | 14 | 0.00933 | 2.05 | 1.15 | 3.65 | 131 | 434 | 435 | 650 |
| C/T or T/T | 10 | 23 | | | | | | | | |
| rs1126973 | Grade 1, 2, 3 | Grade 0 | | | | | | | | |
| Genotype | n = 33 | n = 37 | | | | | | | | |
| T/T or T/C | 33 | 30 | 0.01215 | — | — | — | 12 | 196 | 197 | 531 |
| C/C | 0 | 7 | | | | | | | | |
| rs1126970 | Grade 1, 2, 3 | Grade 0 | | | | | | | | |
| Genotype | n = 33 | n = 37 | | | | | | | | |
| C/C or C/A | 33 | 30 | 0.01215 | — | — | — | 10 | 192 | 193 | 529 |
| A/A | 0 | 7 | | | | | | | | |
| rs1126972 | Grade 1, 2, 3 | Grade 0 | | | | | | | | |
| Genotype | n = 33 | n = 37 | | | | | | | | |
| C/C or C/G | 33 | 30 | 0.01215 | — | — | — | 11 | 194 | 195 | 530 |
| G/G | 0 | 7 | | | | | | | | |
| rs1009668 | Grade 1, 2, 3 | Grade 0 | | | | | | | | |
| Genotype | n = 33 | n = 37 | | | | | | | | |
| T/T or T/C | 10 | 3 | 0.02903 | 1.91 | 1.24 | 2.94 | 3 | 178 | 179 | 522 |
| C/C | 23 | 34 | | | | | | | | |
| rs1145720 | Grade 1, 2, 3 | Grade 0 | | | | | | | | |
| Genotype | n = 33 | n = 37 | | | | | | | | |
| C/C | 13 | 6 | 0.03513 | 1.74 | 1.10 | 2.76 | 16 | 204 | 205 | 535 |
| C/T or T/T | 20 | 31 | | | | | | | | |
| rs2294638 | Grade 1, 2, 3 | Grade 0 | | | | | | | | |
| Genotype | n = 33 | n = 37 | | | | | | | | |
| C/C or C/G | 27 | 21 | 0.03825 | 2.06 | 1.00 | 4.27 | 84 | 340 | 341 | 603 |
| G/G | 6 | 16 | | | | | | | | |
| rs13385 | Grade 1, 2, 3 | Grade 0 | | | | | | | | |
| Genotype | n = 33 | n = 37 | | | | | | | | |
| G/G or G/A | 30 | 26 | 0.03896 | 2.50 | 0.89 | 7.02 | 19 | 210 | 211 | 538 |
| A/A | 3 | 11 | | | | | | | | |
| rs2272615 | Grade 1, 2, 3 | Grade 0 | | | | | | | | |
| Genotype | n = 33 | n = 37 | | | | | | | | |
| G/G or G/A | 15 | 8 | 0.04348 | 1.70 | 1.06 | 2.72 | 73 | 318 | 319 | 592 |
| A/A | 18 | 29 | | | | | | | | |
| rs1884014 | Grade 1, 2, 3 | Grade 0 | | | | | | | | |
| Genotype | n = 33 | n = 37 | | | | | | | | |
| G/G | 6 | 1 | 0.04634 | 2.00 | 1.32 | 3.03 | 40 | 252 | 253 | 559 |
| G/C or C/C | 27 | 36 | | | | | | | | |
| rs2750440 | Grade 1, 2, 3 | Grade 0 | | | | | | | | |
| Genotype | n = 33 | n = 37 | | | | | | | | |
| T/T or T/C | 12 | 5 | 0.04876 | 1.78 | 1.13 | 2.80 | 102 | 376 | 377 | 621 |
| C/C | 21 | 32 | | | | | | | | |
| rs2616023 | Grade 1, 2, 3 | Grade 0 | | | | | | | | |
| Genotype | n = 33 | n = 37 | | | | | | | | |
| G/G or G/A | 12 | 5 | 0.04876 | 1.78 | 1.13 | 2.80 | 96 | 364 | 365 | 615 |
| A/A | 21 | 32 | | | | | | | | |
| rs491528 | Grade 1, 2, 3 | Grade 0 | | | | | | | | |
| Genotype | n = 40 | n = 41 | | | | | | | | |
| G/G | 21 | 11 | 0.02365 | 1.69 | 1.10 | 2.61 | 135 | 442 | 443 | 654 |
| G/T or T/T | 19 | 30 | | | | | | | | |

Next, an SNP typing used in the present invention will be described. A process of SNP typing includes extraction of DNA, amplification of DNA, and DNA sequencing by mass spectroscopy. The SNP typing will be described below in detail.

(Extraction of DNA Sample)

After informed consent is acquired, blood is obtained from cancer patients on whom radiation therapy is to be performed and healthy subjects, and DNA was extracted therefrom. The cancer patients are classified based on cancer type, and a presence of onset of a side-effect was examined in less than 3 months (early stage), at 3 months (late stage of 3 months) and at 6 months (late stage of 6 months) from the beginning of the radiation therapy, and severity of the side-effect was evaluated (see Tables 1-18).

In general, a disorder from a side-effect occurred in less than 3 months from the beginning of the therapy is called “acute disorder (acute effect)”, and a disorder from a side-effect occurred after 3 months from the beginning of the therapy is called “late effect”.

The extraction of DNA from the blood obtained from the patients of various cancer types was performed using NA-3000 manufactured by Kurabo Industries, Ltd., according to a standard protocol attached to the device.

(Selection and Production of Primer)

Various forward primers, reverse primers and extension primers to be used in SNP typing were produced using Primer 3.0 (http://frodo.wi.mit.edu/cgi-bin/primer3/primer3_www.cgi), by selecting an appropriate range for each primer based on information associated with SNP described in the above-mentioned [3]. Synthesis of each primer was entrusted to SIGMA GENOSYS (SIGMA-ALDRICH Japan K.K.) or PROLIGO (Proligo Japan K.K.).

(SNP Typing)

Using the extracted DNA sample, SNP typing was performed by MALDI-TOF/MS. For mass spectrograph, MassARRAY system manufactured by SEQUENOM, Inc. was used. SNP typing by MALDI-TOF/MS was performed in the following processes (1)-(3).

(1) Amplification of DNA:

In order to amplify a base sequence of interest using the DNA sample, PCR was conducted using a DNA oligomer set including DNA oligomers appropriately selected from forward primers and reverse primers shown in SEQ ID NOs: 174-519 in the Sequence Listing.

PCR was conducted under the following conditions. A total amount of 5 μl of a PCR reaction solution was prepared by mixing 0.5 μl of 10× HotStar Taq buffer, 0.2 μl of MgCl₂, 0.04 μl of 25 mM dNTP, 1.0 μl of 1 μM PCR primer, 0.02 μl of HotStar Taq solution, 1.0 μl of DNA solution (2.5 ng/μl) and 2.24 μl of pure water.

The PCR reaction solution was dispensed into wells of a 384-well plate, and PCR was conducted using a thermal circler according to the following scheme: HotStar Taq polymerase activation at 95° C. for 15 minutes, (a) thermal denaturation of double-stranded DNA at 95° C. for 20 seconds, (b) annealing at 56° C. for 30 seconds, and (c) elongation at 72° C. for 1 minutes. A cycle of (a)-(c), in this order, was repeated 55 times, and then elongation reaction was conducted at 72° C. for 3 minutes.

(2) SAP Reaction:

For dephosphorylation reaction of DNA, SAP (Shrimp Alkaline Phosphatase) reaction was conducted. A total amount of 2.0 μl of a SAP reaction solution was prepared by mixing 0.17 μl of hME buffer, 0.3 μl of SAP solution and 1.53 μl of pure water, and added to the PCR reaction solution in which PCR had been conducted as mentioned above, and a reaction was allowed to proceed at 37° C. for 20 minutes, and then at 85° C. for 5 minutes.

(3) Elongation Reaction:

Next, PCR was conducted using the extension primer of SEQ ID NOs: 520-692 in the Sequence Listing, in order to determine a base type of DNA as SNP. A total amount of 2.0 μl of elongation reaction solution was prepared by mixing 0.2 μl of dNTP/ddNTP mixture, 0.054 μl of 100 μM extension primer, 0.018 μl of Termo Sequenase and 1.728 μl of pure water, and added to the reaction solution (7 μl) that had been prepared by mixing the PCR reaction solution and the SAP reaction solution as mentioned above, and PCR was conducted under the following PCR conditions to thereby obtain PCR product for SNP analysis. First, thermal denaturation of double-stranded DNA was conducted at 94° C. for 2 minutes using a thermal circler. Then, a cycle was repeated 55 times, which includes: (a) thermal denaturation of double-stranded DNA at 94° C. for 5 seconds, (b) annealing at 52° C. for 5 seconds, and (c) elongation at 72° C. for 5 seconds.

(4) Desalting Treatment:

Afterwards, 3 mg of SpectroCREAN as desalinating resin and 16 μl of pure water were added per 9 ml of a reaction solution, and a mixture was incubated at room temperature for 10 minutes, to thereby desalt the reaction solution.

(5) Mass Spectroscopy:

The resultant reaction solution for SNP typing was obtained in a total amount of 25 μl. Approximately 0.01 μl of the reaction solution was spotted on Spectro Chip for mass spectroscopy, to perform mass spectroscopy, thus to perform SNP typing of a risk allele (121st base) of the DNA oligomer of SEQ ID NOs: 1-173 in the Sequence Listing.

### INDUSTRIAL APPLICABILITY

(Application to Clinical Use)

The DNA oligomer, genetic marker, DNA oligomer set and method for predicting onset of a side-effect of the present invention may be applied to clinical use, in the following manner.

For example, after informed consent is acquired regarding DNA testing from a cancer patient on whom radiation therapy is to be performed, DNA is extracted from a specimen, such as blood obtained from the patient, and amplified and analyzed using the DNA oligomer set, to thereby determine a base at the SNP site. The determined base is compared with the DNA sequence of the DNA oligomer of the present invention, or SNP typing is conducted using a genetic marker labeled with fluorescence and the like. Based on the above-mentioned relevant risk allele or combinations of risk alleles, a possibility of onset of a side-effect from radiation therapy in this patient is calculated. An attending physician of radiation therapy of the cancer patient can be utilize the calculated possibility as a criterion, to appropriately carry out a treatment regimen with maintaining QOL of the cancer patient, including a care plan after the treatment. In addition, in clinical study of dosage increase, it becomes possible to avoid patients with a high possibility of onset of a side-effect.

As described above, by using the DNA oligomer, genetic marker, DNA oligomer set and method for predicting onset of a side-effect of the present invention, a prediction with scientific basis can be provided against a risk of onset of a side-effect from radiation, against which no prediction means has not been provided to date.

1. A DNA oligomer for predicting a possibility of onset of a side-effect from radiation therapy for cancer by determining whether a specific base in a DNA sequence is a risk allele or a non-risk allele,
the DNA oligomer having a DNA sequence of at least 10-241 contiguous bases with a 121st base from a sequence of any one of SEQ ID NOs: 1-173 in the Sequence Listing.

2. A DNA oligomer for predicting a possibility of onset of a side-effect from radiation therapy for breast cancer by determining whether a specific base in a DNA sequence is a risk allele or a non-risk allele,
the DNA oligomer having a DNA sequence of at least 10-241 contiguous bases with a 121st base from a sequence of SEQ ID NO: 1, 4, 7, 13, 15, 17, 19, 20, 26, 27, 30, 32, 44, 45, 48, 49, 50, 51, 53, 54, 58, 59, 60, 61, 62, 63, 65, 67, 73, 74, 77, 78, 82, 85, 88, 90, 91, 94, 97, 98, 106, 108, 112, 113, 116, 117, 126, 127, 132, 133, 136, 137, 138, 140, 143, 145, 147, 148, 151, 157, 159, 160, 162, 163, 165, 167, 170, 172 or 173 in the Sequence Listing.

3. A DNA oligomer for predicting a possibility of onset of a side-effect from radiation therapy for cervical cancer by determining whether a specific base in a DNA sequence is a risk allele or a non-risk allele,
the DNA oligomer having a DNA sequence of at least 10-241 contiguous bases with a 121st base from a sequence of SEQ ID NO: 2, 6, 22, 23, 29, 31, 34, 36, 37, 39, 41, 42, 43, 44, 46, 52, 56, 60, 64, 65, 68, 70, 71, 72, 75, 76, 80, 83, 86, 89, 91, 93, 98, 105, 110, 114, 118, 119, 121, 129, 134, 139, 141, 142, 144, 146, 149, 150, 152,153, 154, 155, 157, 161 or 171 in the Sequence Listing.

4. A DNA oligomer for predicting a possibility of onset of a side-effect from radiation therapy for prostate cancer by determining whether a specific base in a DNA sequence is a risk allele or a non-risk allele,
the DNA oligomer having a DNA sequence of at least 10-241 contiguous bases with a 121st base from a sequence of SEQ ID NO: 3, 5, 8, 9, 10, 11, 12, 14, 16, 18, 19, 21, 24, 25, 28, 33, 35, 38, 39, 40, 45, 47, 48, 55, 57, 66, 69, 73, 79, 81, 84, 87, 92, 95, 96, 99, 100, 101, 102, 103, 104, 107, 109, 111, 115, 116, 120, 122, 123, 124, 125, 126, 128, 130, 131, 135, 151, 156, 158, 160, 164, 166, 168 or 169 in the Sequence Listing.

5. A DNA oligomer for predicting a possibility of onset of a side-effect from radiation therapy for cancer during a period from a beginning of the therapy to an early stage, by determining whether a specific base in a DNA sequence is a risk allele or a non-risk allele,
the DNA oligomer having a DNA sequence of at least 10-241 contiguous bases with a 121st base from a sequence of SEQ ID NO: 2, 5, 7, 8, 13, 15, 17, 19, 20, 22, 23, 24, 25, 26, 27, 28, 29, 31, 32, 33, 34, 36, 43, 44, 45, 48, 52, 56, 59, 60, 61, 64, 65, 66, 70, 71, 72, 73, 75, 76, 78, 80, 81, 86, 89, 90, 91, 92, 93, 94, 96, 98, 99, 102, 103, 105, 106, 112, 113, 114, 117, 118, 120, 121, 126, 127, 129, 132, 134, 137, 138, 140, 141, 143, 144, 146, 147, 149, 150, 151, 152, 153, 154, 157, 158, 160, 162, 163, 165, 167, 168, 169 or 171 in the Sequence Listing.

6. A DNA oligomer for predicting a possibility of onset of a side-effect from radiation therapy for cancer during a late stage of 3 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 5, 8, 10, 11, 12, 18, 21, 33, 45, 48, 49, 53, 55, 58, 69, 77, 87, 100, 101, 104, 108, 109, 111, 115, 116, 122, 123, 124, 125, 126, 128, 133, 136, 145, 148, 151, 156, 159, 160, 162 or 170 in the Sequence Listing.

7. A DNA oligomer for predicting a possibility of onset of a side-effect from radiation therapy for cancer during a late stage of 6 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 1, 3, 4, 5, 6, 9, 10, 11, 12, 14, 16, 19, 30, 35, 37, 38, 39, 40, 41, 42, 46, 47, 50, 51, 54, 57, 60, 62, 63, 67, 68, 73, 74, 79, 82, 83, 84, 85, 88, 95, 96, 97, 102, 107, 110, 119, 130, 131, 135, 139, 142, 155, 161, 164, 166, 172 or 173 in the Sequence Listing.

8. A DNA oligomer for predicting a possibility of onset of a side-effect from radiation therapy for breast cancer during a period from a beginning of the therapy to an early stage, by determining whether a specific base in a DNA sequence is a risk allele or a non-risk allele,
the DNA oligomer having a DNA sequence of at least 10-241 contiguous bases with a 121st base from a sequence of SEQ ID NO: 7, 13, 15, 17, 19, 20, 26, 27, 32, 44, 45, 59, 61, 65, 73, 78, 90, 91, 94, 98, 106, 112, 113, 117, 127, 132, 137, 138, 140, 143, 147, 157, 160, 162, 163, 165 or 167 in the Sequence Listing.

9. A DNA oligomer for predicting a possibility of onset of a side-effect from radiation therapy for breast cancer during a late stage of 3 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 48, 49, 53, 58, 77, 108, 116, 126, 133, 136, 145, 148, 151, 159, 162 or 170 in the Sequence Listing.

10. A DNA oligomer for predicting a possibility of onset of a side-effect from radiation therapy for breast cancer during a late stage of 6 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 1, 4, 30, 50, 51, 54, 60, 62, 63, 67, 74, 82, 85, 88, 97, 172 or 173 in the Sequence Listing.

11. A DNA oligomer for predicting a possibility of onset of a side-effect from radiation therapy for cervical cancer from a beginning of the therapy to an early stage, by determining whether a specific base in a DNA sequence is a risk allele or a non-risk allele,
the DNA oligomer having a DNA sequence of at least 10-241 contiguous bases with a 121st base from a sequence of SEQ ID NO: 2, 22, 23, 29, 31, 34, 36, 43, 44, 52, 56, 60, 64, 65, 70, 71, 72, 75, 76, 80, 86, 89, 91, 93, 98, 105, 114, 118, 121, 129, 134, 141, 144, 146, 149, 150, 152, 153, 154, 157 or 171 in the Sequence Listing.

12. A DNA oligomer for predicting a possibility of onset of a side-effect from radiation therapy for cervical cancer during a late stage of 6 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 6, 37, 39, 41, 42, 46, 68, 83, 110, 119, 139, 142, 155 or 161 in the Sequence Listing.

13. A DNA oligomer for predicting a possibility of onset of a side-effect from radiation therapy for prostate cancer during a period from a beginning of the therapy to an early stage, by determining whether a specific base in a DNA sequence is a risk allele or a non-risk allele,
the DNA oligomer having a DNA sequence of at least 10-241 contiguous bases with a 121st base from a sequence of SEQ ID NO: 5, 8, 24, 25, 28, 33, 48, 66, 81, 92, 96, 99, 102, 103, 120, 126, 151, 158, 168 or 169 in the Sequence Listing.

14. A DNA oligomer for predicting a possibility of onset of a side-effect from radiation therapy for prostate cancer during a late stage of 3 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 5, 8, 10, 11, 12, 18, 21, 33, 45, 48, 55, 69, 87, 100, 101, 104, 109, 111, 115, 116, 122, 123, 124, 125, 128, 156 or 160 in the Sequence Listing.

15. A DNA oligomer for predicting a possibility of onset of a side-effect from radiation therapy for prostate cancer during a late stage of 6 months from a beginning of the therapy, by determining whether a specific base in a DNA sequence is a risk allele or a non-risk allele,
the DNA oligomer having a DNA sequence of at least 10-241 contiguous bases with a 121st base from a sequence of SEQ ID NO: 3, 5, 9, 10, 11, 12, 14, 16, 19, 35, 38, 39, 40, 47, 57, 73, 79, 84, 95, 96, 102, 107, 130, 131, 135, 164 or 166 in the Sequence Listing.

16. The DNA oligomer for a prediction of onset of a side-effect from radiation therapy according to claim 1, wherein the DNA oligomer includes deletion, substitution or insertion of one to several bases except for the 121 st base, or the DNA oligomer has a complementary DNA sequence thereto.

17. A genetic marker for a prediction of onset of a side-effect from radiation therapy, which is the DNA oligomer for a prediction of onset of a side-effect according to claim 1, or a DNA oligomer that hybridizes with the DNA oligomer for a prediction of onset of a side-effect under stringent conditions.

18. A DNA oligomer set consisting of a pair of DNA oligomers sequentially selected from DNA oligomers of SEQ ID NOs: 174-519 in the Sequence Listing, the SEQ ID NOs of the pair starting from even number.

19. The DNA oligomer set according to claim 1, wherein the DNA oligomers of SEQ ID NOs: 174-519 in the Sequence Listing include deletion, substitution or insertion of one to several bases.

20. A DNA oligomer having a DNA sequence of SEQ ID NOs: 520-692 in the Sequence Listing, optionally including deletion, substitution or insertion of one to several bases.

21. A method for predicting onset of a side-effect from radiation therapy in which determination is made using a DNA oligomer of any one of SEQ ID NOs: 1-173 in the Sequence Listing, comprising the following processes (a)-(g):
(a) a DNA sample is prepared from a specimen obtained from a cancer patient on whom radiation therapy is to be performed;
(b) DNA is amplified from the DNA sample prepared in the process (a) to obtain a DNA product;
(c) elongation reaction is performed using the DNA product amplified in the process (b) as a template, to obtain a DNA oligomer as elongation product;
(d) a DNA sequence of the DNA oligomer obtained in the process (c) is determined;
(e) a comparison is made between a base corresponding to a base at a 121st position of the DNA sequence of the DNA oligomer sequenced in the process (d) and a 121st base of the DNA sequence of any one of SEQ ID NOs: 1-173 in the Sequence Listing;
(f) it is determined whether the allele having the base compared in the process (e) is a risk allele or a non-risk allele; and
(g) a risk rate of onset of a side-effect from radiation in the cancer patient on whom the radiation therapy is to be performed is predicted, based on the result determined in the process (f).

22. The method for predicting onset of a side-effect from radiation therapy according to claim 21, wherein the DNA sequence of any one of SEQ ID NOs: 1-173 in the Sequence Listing to be used for the comparison in the process (e) is:
a DNA sequence of the DNA oligomer of SEQ ID NO: 1, 4, 7, 13, 15, 17, 19, 20, 26, 27, 30, 32, 44, 45, 48, 49, 50, 51, 53, 54, 58, 59, 60, 61, 62, 63, 65, 67, 73, 74, 77, 78, 82, 85, 88, 90, 91, 94, 97, 98, 106, 108, 112, 113, 116, 117, 126, 127, 132, 133, 136, 137, 138, 140, 143, 145, 147, 148, 151, 157, 159, 160, 162, 163, 165, 167, 170, 172 or 173 in the Sequence Listing, for a prediction of onset of a side-effect from radiation therapy for breast cancer;
a DNA sequence of the DNA oligomer of SEQ ID NO: 2, 6, 22, 23, 29, 31, 34, 36, 37, 39, 41, 42, 43, 44, 46, 52, 56, 60, 64, 65, 68, 70, 71, 72, 75, 76, 80, 83, 86, 89, 91, 93, 98, 105, 110, 114, 118, 119, 121, 129, 134, 139, 141, 142, 144, 146, 149, 150, 152, 153, 154, 155, 157, 161 or 171 in the Sequence Listing, for a prediction of onset of a side-effect from radiation therapy for cervical cancer;
a DNA sequence of the DNA oligomer of SEQ ID NO: 3, 5, 8, 9, 10, 11, 12, 14, 16, 18, 19, 21, 24, 25, 28, 33, 35, 38, 39, 40, 45, 47, 48, 55, 57, 66, 69, 73, 79, 81, 84, 87, 92, 95, 96, 99, 100, 101, 102, 103, 104, 107, 109, 111, 115, 116, 120, 122, 123, 124, 125, 126, 128, 130, 131, 135, 151, 156, 158, 160, 164, 166, 168 or 169 in the Sequence Listing, for a prediction of onset of a side-effect from radiation therapy for prostate cancer;
a DNA sequence of the DNA oligomer of SEQ ID NO: 2, 5, 7, 8, 13, 15, 17, 19, 20, 22, 23, 24, 25, 26, 27, 28, 29, 31, 32, 33, 34, 36, 43, 44, 45, 48, 52, 56, 59, 60, 61, 64, 65, 66, 70, 71, 72, 73, 75, 76, 78, 80, 81, 86, 89, 90, 91, 92, 93, 94, 96, 98, 99, 102, 103, 105, 106, 112, 113, 114, 117, 118, 120, 121, 126, 127, 129, 132, 134, 137, 138, 140, 141, 143, 144, 146, 147, 149, 150, 151, 152, 153, 154, 157, 158, 160, 162, 163, 165, 167, 168, 169 or 171 in the Sequence Listing, for a prediction of onset of a side-effect from radiation therapy for cancer during a period from a beginning of the therapy to an early stage;
a DNA sequence of the DNA oligomer of SEQ ID NO: 5, 8, 10, 11, 12, 18, 21, 33, 45, 48, 49, 53, 55, 58, 69, 77, 87, 100, 101, 104, 108, 109, 111, 115, 116, 122, 123, 124, 125, 126, 128, 133, 136, 145, 148, 151, 156, 159, 160, 162 or 170 in the Sequence Listing, for a prediction of onset of a side-effect from radiation therapy for cancer during a late stage of 3 months from a beginning of the therapy;
a DNA sequence of the DNA oligomer of SEQ ID NO: 1, 3, 4, 5, 6, 9, 10, 11, 12, 14, 16, 19, 30, 35, 37, 38, 39, 40, 41, 42, 46, 47, 50, 51, 54, 57, 60, 62, 63, 67, 68, 73, 74, 79, 82, 83, 84, 85, 88, 95, 96, 97, 102, 107, 110, 119, 130, 131, 135, 139, 142, 155, 161, 164, 166, 172 or 173 in the Sequence Listing, for a prediction of onset of a side-effect from radiation therapy for cancer during a late stage of 6 months from a beginning of the therapy;
a DNA sequence of the DNA oligomer of SEQ ID NO: 7, 13, 15, 17, 19, 20, 26, 27, 32, 44, 45, 59, 61, 65, 73, 78, 90, 91, 94, 98, 106, 112, 113, 117, 127, 132, 137, 138, 140, 143, 147, 157, 160, 162, 163, 165 or 167 in the Sequence Listing, for a prediction of onset of a side-effect from radiation therapy for breast cancer during a period from a beginning of the therapy to an early stage;
a DNA sequence of the DNA oligomer of SEQ ID NO: 48, 49, 53, 58, 77, 108, 116, 126, 133, 136, 145, 148, 151, 159, 162 or 170 in the Sequence Listing, for a prediction of onset of a side-effect from radiation therapy for breast cancer during a late stage of 3 months from a beginning of the therapy;
a DNA sequence of the DNA oligomer of SEQ ID NO: 1, 4, 30, 50, 51, 54, 60, 62, 63, 67, 74, 82, 85, 88, 97, 172 or 173 in the Sequence Listing, for a prediction of onset of a side-effect from radiation therapy for breast cancer during a late stage of 6 months from a beginning of the therapy;
a DNA sequence of the DNA oligomer of SEQ ID NO: 2, 22, 23, 29, 31, 34, 36, 43, 44, 52, 56, 60, 64, 65, 70, 71, 72, 75, 76, 80, 86, 89, 91, 93, 98, 105, 114, 118, 121, 129, 134, 141, 144, 146, 149, 150, 152, 153, 154, 157 or 171 in the Sequence Listing, for a prediction of onset of a side-effect from radiation therapy for cervical cancer during a period from a beginning of the therapy to an early stage;
a DNA sequence of the DNA oligomer of SEQ ID NO: 6, 37, 39, 41, 42, 46, 68, 83, 110, 119, 139, 142, 155 or 161 in the Sequence Listing, for a prediction of onset of a side-effect from radiation therapy for cervical cancer during a late stage of 6 months from a beginning of the therapy;
a DNA sequence of the DNA oligomer of SEQ ID NO: 5, 8, 24, 25, 28, 33, 48, 66, 81, 92, 96, 99, 102, 103, 120, 126, 151, 158, 168 or 169 in the Sequence Listing, for a prediction of onset of a side-effect from radiation therapy for prostate cancer during a period from a beginning of the therapy to an early stage;
a DNA sequence of the DNA oligomer of SEQ ID NO: 5, 8, 10, 11, 12, 18, 21, 33, 45, 48, 55, 69, 87, 100, 101, 104, 109, 111, 115, 116, 122, 123, 124, 125, 128, 156 or 160 in the Sequence Listing, for a prediction of onset of a side-effect from radiation therapy for prostate cancer during a late stage of 3 months from a beginning of the therapy; and
a DNA sequence of the DNA oligomer of SEQ ID NO: 3, 5, 9, 10, 11, 12, 14, 16, 19, 35, 38, 39, 40, 47, 57, 73, 79, 84, 95, 96, 102, 107, 130, 131, 135, 164 or 166 in the Sequence Listing, for a prediction of onset of a side-effect from radiation therapy for prostate cancer during a late stage of 6 months from a beginning of the therapy.


SEQUENCE LISTING (693 sequences)

SEQ ID NO: 1 - DNA, 241 bp, Homo sapiens
  AB183822 (121)..(121): SNP(121A to G)
gatgaagttcttattctgagtacagtactctttgtcatttcatattggattttctatagagaagaagcacaatggggaagataggagcaaggtcatgtaccctaatagttactatgttttataaatccattttgtagagggcatgtaaataaatgttttcctgtagtcatagattattcaggactgtcctttagttctgtcttttgaactcatgggaataattgtgagtcagcgtaacatt

SEQ ID NO: 2 - DNA, 241 bp, Homo sapiens
  rs1002481 (121)..(121): SNP(121T to A)
tcataactacagaactgataaaatgtatgaatgatcaacagtgggatcatagctaggaaaagtaatgatatcaccaggtcattaatttttatgagaactacaaaggattgtttcccagtctggatctttgactcaaaagcatttgcttttcatcagtgccccaaaagagaagataaagaagacaccacataattgaccctacttaacagaatgaagagataacatctagtgtggatcctag

SEQ ID NO: 3 - DNA, 241 bp, Homo sapiens
  rs1009668 (121)..(121): SNP(121T to C)
tgatagctgtccccgagaggtttggtgcaatcagccgtccatgtacccatgtaccctgggacaggctccagggccactcaccaaatggcgcgatgatgggacaagtgatgctgtaggccatgatgacagtgaagacacacagcatccatgcatacatggctccaaactcgtactggaaggcctggttctgggaggaggaggtggtgaggagctcatggacttgttccacctcaaacaccct

SEQ ID NO: 4 - DNA, 241 bp, Homo sapiens
  rs102275 (121)..(121): SNP(121C to T)
tataccacctttccaactgtagcacccctattccccacctccactccagccaggtcccctgctcactgtcctggaaatccaaagaacaaactgagctcacctgaacttgggaaataaggccataacctgcccctgggcatcactagccctcctctcctccttaccttctctcagccccaatccaatgtacaaaaccggcggggtccatgtacctagcgtggtttctccctgagcttcccac

SEQ ID NO: 5 - DNA, 241 bp, Homo sapiens
  rs1045376 (121)..(121): SNP(121T to C)
tgttcttcactcccttcagccacacgcttcatggccttgagttcaccttggctgtcctaacagggccaatcaccagtgaccagctagactgtgattttgatagcgtcattcagaagaaggtgtccaaggagctgaaggtggtgaaatttgtcctgcaggtccctcgggagatcctggagctggagcatgagtgtctgacaatcagaagcatcatgtccaatgtccagatggccagaatgaa

SEQ ID NO: 6 - DNA, 241 bp, Homo sapiens
  rs1047347 (121)..(121): SNP(121C to A)
ccaggtttattgaggtatgattgacatccagtaaaattcaccctttggaaatatacagctctgtgaattttgacaaatttagtgtcttgtgaccatcaccaagatcaacctgtttttaaccctccaaaaaattcccttctcctgccctctttccctggcaaccctgattgattatctgatcctataattttgccttttctagaatgtcatataaatggagtcacactgatgtcgccttttg

SEQ ID NO: 7 - DNA, 241 bp, Homo sapiens
  rs1055677 (121)..(121): SNP(121C to G)
tcctcctctgtttcatagaaaagtcatagaatatctatgatacattgaaaagttactgcaatatttgagaactgttctttttttttcttgtgtgtgccatctttccattgttggctacgtcttttcttttgccttgatgaacgttctatgtatttcatcggatatacagcatattccatttaggatgtgtatttaatgcatttagtaatgacgataaagtgtttttagtatgcttttagtc

SEQ ID NO: 8 - DNA, 241 bp, Homo sapiens
  rs1059234 (121)..(121): SNP(121C to T)
cgccgcgtcctcttcttcttggcctggctgacttctgctgtctctcctcagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctgg

SEQ ID NO: 9 - DNA, 241 bp, Homo sapiens
  rs1104893 (121)..(121): SNP(121G to A)
acacacacaaaaggttaagaattcctgccctagaacctaaggaaagccaactatcacctactgccttctcaatatttcagtttggatggcttatatgcatctccatttatgtccaaaactgccctagtgcttcccatctcaggaaatggcccaccattctttgcattacttaggccaaatgctttgtagtcaacttgactcttctcttttgtcccatgttcaatccatccccgaattctaa

SEQ ID NO: 10 - DNA, 241 bp, Homo sapiens
  rs1126970 (121)..(121): SNP(121C to A)
acctcaggcctgggcacctctttgcttgaaatatggcaagacttggaaaaatgtttgcccttagaatctatctcactactttagttagttgtctcctttgggcctgggcacagttctggccctgatctggaacagactcccttttctaaaactgaacttgaccacatcaaaagtttgtaaaacaatctccatggtaattaaacttgcattcaacaccatatggtaacagaagatggcaaag

SEQ ID NO: 11 - DNA, 241 bp, Homo sapiens
  rs1126972 (121)..(121): SNP(121G to C)
acagcaactaattcatcactacctgaggagacaaagtgggtgctgcagaagcaagactgtaggccagtgggatttgtctaataattgatgataagatgatagcacagagggcaacgtactgagagaggaaggcagccttaagaaatgatgcagaaatagccaagcaagatttttccaagcactggttaaatcaccagcaaaacaaagcatttatgtgagtacctcaggcctgggcacctct

SEQ ID NO: 12 - DNA, 241 bp, Homo sapiens
  rs1126973 (121)..(121): SNP(121T to C)
gaaagggcaggttggtcgagaatggaataggaagaactagctgcctcacttttcactttcgcgcctacattttcttcgctccctaagttatacaagatgtacatttcctctcttaagcaatttaatttgggtgctacttccctccttttgtgacagcaactaattcatcactacctgaggagacaaagtgggtgctgcagaagcaagactgtaggccagtgggatttgtctaataattgat

SEQ ID NO: 13 - DNA, 241 bp, Homo sapiens
  rs1144153 (121)..(121): SNP(121C to T)
cactctgtctccaccttgcctgcagcatctttctccaccagcaaggagttccacatgtcaagactttagtgtcggctgcctgcaaatcctgtaaaacgggaagtgcaggggagagatttccgtactcagaggtcagcctagagctctgccatgacctcacagccatagtaagctacatgaaactccagaagagtctcacattacttgcttctaataatggggccctgacatttctaagttc

SEQ ID NO: 14 - DNA, 241 bp, Homo sapiens
  rs1144153 (121)..(121): SNP(121T to C)
cactctgtctccaccttgcctgcagcatctttctccaccagcaaggagttccacatgtcaagactttagtgtcggctgcctgcaaatcctgtaaaacgggaagtgcaggggagagatttctgtactcagaggtcagcctagagctctgccatgacctcacagccatagtaagctacatgaaactccagaagagtctcacattacttgcttctaataatggggccctgacatttctaagttc

SEQ ID NO: 15 - DNA, 241 bp, Homo sapiens
  rs1145720 (121)..(121): SNP(121T to C)
tgaagatttgcacgagtcaaaattaggaaaatgtcttctgatttgcctttgagtctcaagagaatactaagcattgactagcctgcagatcttgttatatttacctgggcttcccttccatggtttgaagcagatccctcagaggctgcctccacccacgtgtgacctctgactgttcaagggaccatatagcaaagacttcagaagcattttggagaatcccaaggccctgagagattgg

SEQ ID NO: 16 - DNA, 241 bp, Homo sapiens
  rs1145720 (121)..(121): SNP(121C to T)
tgaagatttgcacgagtcaaaattaggaaaatgtcttctgatttgcctttgagtctcaagagaatactaagcattgactagcctgcagatcttgttatatttacctgggcttcccttccacggtttgaagcagatccctcagaggctgcctccacccacgtgtgacctctgactgttcaagggaccatatagcaaagacttcagaagcattttggagaatcccaaggccctgagagattgg

SEQ ID NO: 17 - DNA, 241 bp, Homo sapiens
  rs1171097 (121)..(121): SNP(121C to G)
cttaatctagttctgcagacgcccccatcctggcaaagataccgctctgggtctcaccccatgcacagcgccgcatgccaatgccctccctcccgcctgccctccctctctgctttaaggcgatctggggcttttgggctttggttctccacaagcgcttgctccatgttgaccaggtggagagggtctggagcttccaggacctcctcagcgagacatcatcttcacaaactctgcaaag

SEQ ID NO: 18 - DNA, 241 bp, Homo sapiens
  rs1171097 (121)..(121): SNP(121G to C)
cttaatctagttctgcagacgcccccatcctggcaaagataccgctctgggtctcaccccatgcacagcgccgcatgccaatgccctccctcccgcctgccctccctctctgctttaaggggatctggggcttttgggctttggttctccacaagcgcttgctccatgttgaccaggtggagagggtctggagcttccaggacctcctcagcgagacatcatcttcacaaactctgcaaag

SEQ ID NO: 19 - DNA, 241 bp, Homo sapiens
  rs13385 (121)..(121): SNP(121G to A)
ggagttagagaaatgtcttgacacaagggaaagtgagttcccacgctgtgggcctggggccatgggggtgagggaccaggaaagctacaggcatggaagcccaacctcttctgagacttggcagggagaggacacggggagacactgatatctgttacactttgacatcagcaccttgggctttcaaaacacaaatggtagacaaaatcgcctaggcaagactgaagtccaactcatagag

SEQ ID NO: 20 - DNA, 241 bp, Homo sapiens
  rs1339458 (121)..(121): SNP(121T to C)
cagctacaaacatgaatgaaacaagtagccgttcccacgctgtgtttacgattgttttcacccagaagaaacacgataatgagaccaacctttccactgagaaggtaggagagtttcagtttctaggcttgagttgtgaaggatggagattttgagaagtcccttttgttgccctctgctttttgtggaaggttgggtgttggggggtgctcttttacttaatggagggtgttttatacct

SEQ ID NO: 21 - DNA, 241 bp, Homo sapiens
  rs13436 (121)..(121): SNP(121C to G)
gatgggagagagggagaggtcagcgcacttcacctcccacacagcgctggggtccagccagtggtcgggaatcacagcgccatctatccgcacgtaagggcgtgggctgggcagcaccagcgcctgcagtgagcagaggaagagaggaacagagggtctggaatctcaaagtcaaggccaagtgttggagggctggggagagggaggaagggaaaaaaacaggcacccccttgaagtaaac

SEQ ID NO: 22 - DNA, 241 bp, Homo sapiens
  rs1351978 (121)..(121): SNP(121A to C)
acagatgaaaccaaactagcaagacagtagattttaattaaaccatagcagcaattacattaaatgtaaatcatctaaacacatcacttaaaaaacagagactatcaaactaggtgaagaagtgagatccaattttatgctgtccacatgtttttcaggggcaggaataaaagaaatacttaagaatacagaatatgggaaaaatatggtttcccataatttaaaatatttaaaaatagga

SEQ ID NO: 23 - DNA, 241 bp, Homo sapiens
  rs1405655 (121)..(121): SNP(121T to C)
agagttaaggaagaccagtgcttgccgttgtaggatgacattccacggcgaatagagtcttctatgagccaaggagagaaaggaaaaagggtctgaggctgagaaaattgaggatcaggctagaggcgctattgcagagaaaagttgccaaaaaggcttttcagaggagttagcatttgaatcaaagtagggataacatataagtattgagcacgtgctgtatgccaggctctattccaag

SEQ ID NO: 24 - DNA, 241 bp, Homo sapiens
  rs1475489 (121)..(121): SNP(121T to A)
cagtgaaacctgactgtttttttctggtaggtttggtgaagagtggacatctgcggagaaatatgataggacacatagggtttgagctaaatatgataaactcagggaaatttagcaaggtcttttgattctgatttctctctttctacagagataaagatgttcttttctccgagtataaggaggacgcttctcacttgaggctcttatgacctcctttgagggcaggtcagaaaatcct

SEQ ID NO: 25 - DNA, 241 bp, Homo sapiens
  rs1520483 (121)..(121): SNP(121T to C)
cctgccctctatgagcctctagcttcaaaccccaagaagagtgttgggaaggaagtgtgagatctgagtagaactggcttcaggtgggataaggcagaaaaggccttcctgagacatcaattcacgcttggacaatagcttggcagatgggagaccttgggccctgtatcttttccctgccagtggtgagagattgctgacttgagaaccacttgttaaacatttagggatttggggtggg

SEQ ID NO: 26 - DNA, 241 bp, Homo sapiens
  rs153017 (121)..(121): SNP(121A to G)
tctgccactgcacccaggattgcctggatttaaaactcaggctccatcactcagctctctgacctaggtgaagtgacttaactccctcagcctctgtttattcatcagtaaaatattagtactttcctcaaaaagtacgttttctgaaagttaatgagatgataaataatacaagacttagatcagggcctggaaggtcttagcaggtcttaaatgcccaatgaatgttgagatcttaata

SEQ ID NO: 27 - DNA, 241 bp, Homo sapiens
  rs1555025 (121)..(121): SNP(121G to T)
atttctacataaacctttttaagagcccccccccacccaattgggtcagcaaatttcttaacttcccctctcttcactttccacacacctctgtgtagaaaacagtggtaatttcattgcgctattgtctgtccctttctgctatggaagggcaggccacctggacttgattattcttagccctgctgaagtagctccagctccatctgaaagaccaggaaaggaaccagagggccggcaa

SEQ ID NO: 28 - DNA, 241 bp, Homo sapiens
  rs157703 (121)..(121): SNP(121T to C)
ttctgaggagttatagaaactgccttcctccctttttaaagaaacacagcctaatttccttactactgcattcacagttcttaaatatgtatgtatcttcagttttcttaagcatgaccttcatgaaattttatacttaggtaactgaattgttggtggtaactaaaatgaagtgataaggaaaaaattttggcaactttatatatgtatgataataggattcccttcattaattttaatc

SEQ ID NO: 29 - DNA, 241 bp, Homo sapiens
  rs165815 (121)..(121): SNP(121C to T)
aacccctagacacggggcctcaccggggacctgcccacagccacatgaccacttactttcaagggttccttctcagaggcctctcctgcagagctggcgccccgtggcctccgctccctccggtccaccgtggagtatccgtctgtagatggggtaaggtggggcatgaggggcgctctgagggaggccctgtaggagcagatctccctctcagcccagacacaggggactcgtgggacag

SEQ ID NO: 30 - DNA, 241 bp, Homo sapiens
  rs1673041 (121)..(121): SNP(121T to G)
cccagtctccagccacccacacccagccccagaccgtctttctgcccccagggttgctctcgaccccctagggttgttataaggatgttgtggttggtctcaatctccgttcttcaggcttatgtgacggggacccgcagcctgctgcacaccctgcctctcctcctcaggtacaaacattccctttcctgggccgtgtggccggcctttgctccaacatccgggactcttcattccagtc

SEQ ID NO: 31 - DNA, 241 bp, Homo sapiens
  rs1684385 (121)..(121): SNP(121A to G)
aactccagaggatctaactggccaaaagcaatggtacagctctgggcctttgtgcatgctcttctctgttgtgtgcactcctacacatccctgaggccccagctcaggacactccactgaagtccccatcatagccccctggcagaattgactgcctctgtgctccctgcgtagcgcatttatatctattctgcacagatcttgttcttttacacagtagtcccccttatctatagtagat

SEQ ID NO: 32 - DNA, 241 bp, Homo sapiens
  rs170548 (121)..(121): SNP(121A to C)
caacttgaagtcacataggaaaccttgaacttaacggacaagctacatgtaatcaagctcagtttctccacagtatttaatggtcctggaggacactcaaaacagcattaaaaaatagagagattttggttctgctaatctagtacgtgtcctgaaactaaatctgctaaaggcattttatagtcccaacctgcagatttactgagaaactgtatacagatagcagcaaaaaagtgttcca

SEQ ID NO: 33 - DNA, 241 bp, Homo sapiens
  rs1801270 (121)..(121): SNP(121C to A)
ccttccttgtatctctgctgcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggcctt

SEQ ID NO: 34 - DNA, 241 bp, Homo sapiens
  rs1805312 (121)..(121): SNP(121G to C)
cacctgccttggcatacgccaatgccacctcagccagccgctggcggctctcctcagcccggaatgctccgttttcactcaggagcccttcaggacagatgactgggttttggggagcacgttgggccctggcctgtccccaccctgttgagaagtgggcagtaggagtgtgggtggcagcagggctggtgggagggagggaactcaccgcagtgaccatgggaggtgtagggacacaggc

SEQ ID NO: 35 - DNA, 241 bp, Homo sapiens
  rs1805414 (121)..(121): SNP(121G to A)
cttgaccatacacttggtccaggcagtgacgtccccagtgcagtaataggcatcgctcttgaagaccagctgacccgagcattcctcgcagggaaggagggcaccgaacaccatgccatcggctactcggtccaagatctgcagccagtggagaaacatgtcagagggcaaatgcgggactacaaaaaaggacacttactaatgtgggatggaggaacttgcacagattccactcactaca

SEQ ID NO: 36 - DNA, 241 bp, Homo sapiens
  rs183460 (121)..(121): SNP(121C to A)
ataaggttgtaacagaagtacaatgctatggcaacatttaagaagtactgactgtacttgctgaatgaagtttcagagatcaggcatgacctgaaccttgaatagaagtgtactaggaagccaaagagaagtagaatacatacaagaattttcaaagaaacttcaattcgtttggcatgtctgcagcataaaacagatgttaggaagttgagatcaagttggagaggtaggtaggatcagt

SEQ ID NO: 37 - DNA, 241 bp, Homo sapiens
  rs1845452 (121)..(121): SNP(121C to G)
aagctataatcagggtaagggatattgtagtgcttgaaacctgtgaggctgagagggtctactttgcattcttgttcgttagcaacccatttcacatcttcctccgagttccccagcaccccacagagctactctcagtggcacaaagctctattaagtaaatagaggctccatatttaaaagccatctctgggaattcaaagtgctctgagagggaagtcagtactatcaagattaaggt

SEQ ID NO: 38 - DNA, 241 bp, Homo sapiens
  rs1862391 (121)..(121): SNP(121G to T)
aataacaatggtttattgctttaagatatttttatttagatgaccctgtaaaaaggatggaaaataaatagaaatattgaagaaataggggaaacaaccaatgttgggtctaaagaatacgagcagagaagaatttaaaattaagaagaaatgtaagtacaggtagatcaaacagcagcgtttgagtcattctcttatgcttatgattaagaacgaactgatcgttccctaatcttaggac

SEQ ID NO: 39 - DNA, 241 bp, Homo sapiens
  rs1862392 (121)..(121): SNP(121A to T)
caagcaaaatttcttttcatatctgggaaattggaattccttttttatatctttattatctctctgatattctcttgctccctggccaagcccttccaggcaactttggttaatatattaatgagatgttagaggtttaaaataaatcactatcaaaggatagaattttaagggaagactaaattatgtattcattagaatcataattttgttcctaagacagattcgtgatggtataata

SEQ ID NO: 40 - DNA, 241 bp, Homo sapiens
  rs1884014 (121)..(121): SNP(121G to C)
tatctcaagataaaagaaagaaagaaaaaaaagtccgtagcctaaattagccgtcctgtgatcttctagtaccaagaccgttgtagaatatagcttcagagctagaaagagcattaggtggttacaattgctaatccctatgttgccatgtggagggacatcagctgtccttcactcctggcctctttctccacctgtccccattgagttatctgatccccgagtttggaaggaactgaac

SEQ ID NO: 41 - DNA, 241 bp, Homo sapiens
  rs190246 (121)..(121): SNP(121T to G)
gcctcaggagttcctctgtgtatctctggtccagattccggaggaaacctgtttgacttgagtagatgccattgcccttattgggccttgcctcaggagttgctggttagcctatgaaggtgttccctttgggttgatgacattgctcccatccagaaagtggctacttctaccctcctactcccaccactctgcatgcaggagctgttatcatcaaaagggaatgtccactgtctgctct

SEQ ID NO: 42 - DNA, 241 bp, Homo sapiens
  rs1928156 (121)..(121): SNP(121A to G)
acttcagcatcggtttttcatggcagaaacttaatcagataagaacatgagtcagcaaaaaaattatcttcatggaaatagtgctcttcactacaaaagtgcattcactgaatgttttatatcatagactaatatgtcacatcgatgttcttagatgatctttctcagaagaaactgcagaagcaatttggtttcatttatgggaataaataagggtatttcatttcacacagcctcagga

SEQ ID NO: 43 - DNA, 241 bp, Homo sapiens
  rs1968415 (121)..(121): SNP(121T to C)
ctcccaggataaagactcacctcatcttctatctttaagggcttgcggatttcactgtccaaatctctccctctttcacactctgacccatctacactggcccatcttctctttaccttatgggctgagctcttttccaccccagggttctgtgtgttatttggtgaatcaattaatgaattaattcagcttcccgtcactaaccagatagcccacatctaagaatctaatgataaaacag

SEQ ID NO: 44 - DNA, 241 bp, Homo sapiens
  rs1971783 (121)..(121): SNP(121T to C)
gtcttctactgtaaaattcatatgctttgctactctaaacctagtttgaaatcaacagtcttgagaatagatgaaaattttgatgaatagtggaattcttttaaatggaaacctcttacatgtgattttccttgccatctagaaataaaccatagtatttatgttgaatcaatcaatattatattttgtttttttcctcctcttctgagactcttattgtggaaatgttagacttttatgt

SEQ ID NO: 45 - DNA, 241 bp, Homo sapiens
  rs2007182 (121)..(121): SNP(121G to A)
gggccgcgaccacggcgtcctggacgccctgcacgtgctcgagggcctcttccgcaccgaccccgaggccaaggtggggtctcgagagaggcaggccgggggagggggcgggggaaagccgcgctgaagactcgtgggtcattcatgccttcatccgccctctcatcccttcactcctttcctccattcatccgttcattcattcaataagtacttaccgaggcattggggatgcagcagc

SEQ ID NO: 46 - DNA, 241 bp, Homo sapiens
  rs2008521 (121)..(121): SNP(121T to C)
tgtcttggagatcatcgagcacccctttcaggtcccccatcctggcttggagttgccggtggttctccttcacaaaggctgcacctggatgaaagggggagtttcagactttgagaatcgttcacttacaggattcaggatctataccaagcttagagtcaaaattcaggggtgggagattgctccttgtgtctgtagcttaatttgtggacatacaaaatcacacacattcacagagcaa

SEQ ID NO: 47 - DNA, 241 bp, Homo sapiens
  rs2010352 (121)..(121): SNP(121G to A)
gaggtagaaatcagaggagtgggcaagtatagtcaaggatgctttcagagcacgtgctttaaacatactaatttagagctgtaaacatctgggaactatccagatggttgtctaataggtgtctagaaattgggctgttttaattataaatttaagtcatcagcacaagggagtgaatagtcctgaatgagtgagattactagtgggaggggcagaattacaatatggctgcaatgaaatc

SEQ ID NO: 48 - DNA, 241 bp, Homo sapiens
  rs2066505 (121)..(121): SNP(121A to G)
atacccactctgggctgggaggatcacatggctcaaacatcagctgtttaagtggcttcttataatctcattaccaagagaagggatttaagccttgttccaggttgggaacagcccaaaatagcatcttttctcctctaggaccccagcaaaatacccagctggttgaaggtcaacaagatctactatcctgacttcatcgtcccagacccaaaggtatcaacccagtggcttgggggcc

SEQ ID NO: 49 - DNA, 241 bp, Homo sapiens
  rs2071010 (121)..(121): SNP(121A to G)
ctatggcattcccaccatgtgttaaggattttctgaactggaagggccctctgtttgcctgaaggccagagaatcttgaagtggagactgaggcccagaccagagtgtggcctgctcaagattaaacgacaagttagtgttcatccccctgaactagtacctgggctctagcccttcagtccagagctgagttctcagctcttctagtctggggccccaaggttgggtgtgggggtcatga

SEQ ID NO: 50 - DNA, 241 bp, Homo sapiens
  rs2071863 (121)..(121): SNP(121G to A)
accatgctgcctttctcagagcacatctacaaggagattgtgaacttcagccccattgcccggaaggactcacggcgccggagtggcatgaagctgtagggactcatcttgcatggcaccgccggccagacactgcccaaggaccagtatttgtcactaccaaactcagcccttcttggaatacagcctttcaagcagaggacagaagggtccttctccttatgtgggaaatgggcctagt

SEQ ID NO: 51 - DNA, 241 bp, Homo sapiens
  rs2072817 (121)..(121): SNP(121C to T)
acaggaggaatgaaacagactgagtttactctgtcttctgtaactgagttactcctgtcttccactaattcaaaggcctcaattcacggtagtggagtaaaatacacttcctgcagcactcgctatagcacacggcaccaccgcaagcacaaatgatgggggaaggagttactgcttcagagaagattgggaatcactgttctcatgagtgacagagtattttcttttcacaattgctgct

SEQ ID NO: 52 - DNA, 241 bp, Homo sapiens
  rs2072817 (121)..(121): SNP(121T to C)
acaggaggaatgaaacagactgagtttactctgtcttctgtaactgagttactcctgtcttccactaattcaaaggcctcaattcacggtagtggagtaaaatacacttcctgcagcacttgctatagcacacggcaccaccgcaagcacaaatgatgggggaaggagttactgcttcagagaagattgggaatcactgttctcatgagtgacagagtattttcttttcacaattgctgct

SEQ ID NO: 53 - DNA, 241 bp, Homo sapiens
  rs2073495 (121)..(121): SNP(121G to C)
caccacggcatgattcggggccatggcaagatggcagcttctagcctcagtcacaggcttccgtttgccatcgaggcacagcagcgcaaagtctgccagcttcaaatccttagcccatgcgtcattgttatttcctggggagaaaaagaaggtggcatcatccacgcctcccctgcttagccaagaagtctcttctgggtgggtgtggccattccactgacctcccaaatgcacacactgg

SEQ ID NO: 54 - DNA, 241 bp, Homo sapiens
  rs2073747 (121)..(121): SNP(121G to A)
gccatcaccagggcctgggccaagggggccagcacgtgggggccgcatgcccagccctcgggacaggctgccatagctggggctgtcccggggctcggggccttcgggaaagccacccccgctgctgaggtaggctcgagagagtgtggccactgggccaccaccacgcagcaggaaatgccggtccagggcaccatctgcaaaagggcctagtggggggccgccatccagcagggggagt

SEQ ID NO: 55 - DNA, 241 bp, Homo sapiens
  rs2075747 (121)..(121): SNP(121G to A)
ttttcttgtctgtaaagtgggtgtggtaataccttcttcccaggttatgataacctggcccacataggtgttcagctagtagcacctcttattacagcaatttcagtgtattcataggccgcgcatggtggctcacgcctgtaatcccagcactttgggaggccaaggtggacagatcacttgaggtcaggagttcgagaccaggctggccaacatggtgaaaccccatctctactaaaaa

SEQ ID NO: 56 - DNA, 241 bp, Homo sapiens
  rs2075784 (121)..(121): SNP(121A to G)
accccagtcaggcgcggcgagtgcgggcggctgcgggaaccgggcctccctcccgcctcggcggcgccagccgaggacggccccatggcaccctccggagggccggcccgagagcctcagaagggcgcccctcacctgctggcctcgccatccgcgcctgggtccgcgcgccgccgcccgccgctcctcagagacatggagccgttggctaccacctctgcttcaggggagaaatttggcg

SEQ ID NO: 57 - DNA, 241 bp, Homo sapiens
  rs2190935 (121)..(121): SNP(121T to C)
agagaatattgtttcatttaaaaatactttccacagtttcacttggtttcaccaatttcaattggttttaaatcattaggatgtctaagtaaactattgatttcttccacaggtctagtatattaggttcctctgctttaccttgccatctttaatgtttgtattcttttaaaattagctggtcctttataagtatgtaaatattgtagctagcaatgtgctgtttattaacaatacctgt

SEQ ID NO: 58 - DNA, 241 bp, Homo sapiens
  rs2229688 (121)..(121): SNP(121A to G)
attcctctaacatggttctctcctgtgttctgcatgtaggcctttgaggattggaataagacagagctagactcattcctgattgaaatcacagccaatattctcaagttccaagacaccaatggcaaacacctgctgccaaagatcagggacagcgcggggcagaagggcacagggaagtggaccgccatctccgccctggaatacggcgtacccgtcaccctcattggtaatgttatgc

SEQ ID NO: 59 - DNA, 241 bp, Homo sapiens
  rs2232242 (121)..(121): SNP(121A to G)
gtgcagcagtgcggtgaggcgagggtgcgaggcggctaagcaagggatgggcccgccgtggcgagccgctgggccgtgttccgcctcctttcgggctttccgcgggtgtctggggagggaacccggccgcgggcctcaggccgtccccctccgcagaccccttctcttattctgagtctaacctatccgctggggccctggaaagcgattctcgcctggagtggctttaggcagcggtggt

SEQ ID NO: 60 - DNA, 241 bp, Homo sapiens
  rs2238780 (121)..(121): SNP(121G to C)
tgagttggcagataaggacatagaccctggggggtgaagtcttcagggccctccccaagctgcatggcctggctgccacaggctcctccaggccccaggaggtgccagtgagacctgcaggatgccttcctgatgtcaaccaccacccctagctgggacatcatggtgtcctggggcctcctccctcacctgccccagacaccttgagtatagaagccttgtgggggctgtttcagcactg

SEQ ID NO: 61 - DNA, 241 bp, Homo sapiens
  rs2239393 (121)..(121): SNP(121G to A)
gagtgggccatgaacgtgggcgacaagaaaggtggggtccgggccagcaggtgctcagctctgggacagggacccaggaccaggcatcaaagcccttacaggagaagctgttatcaccccgtttccagggggctgggaaccctgggatatgcccagatagggctggggggctcctctggagtcccagggtgccagggtccctgatgacccctgcaggccctgctgcctgctgccccaggac

SEQ ID NO: 62 - DNA, 241 bp, Homo sapiens
  rs2240718 (121)..(121): SNP(121G to T)
cactcatcacccccccatgccctcccggaagcctcaggttcccactgcacaccctggatcaagcaggcagcatgtggaacctcatcccatcacttctgggcactgagcacccaccagggggacaagaagagggaggcctggggtcagtggggcaggggcaggtggcgtacaggtcccccacagctgctggggaggtcctgggaacctacaccctgctagtgcagtagagaaggtgtgcagg

SEQ ID NO: 63 - DNA, 241 bp, Homo sapiens
  rs2241502 (121)..(121): SNP(121G to A)
tattaaaatgggaagttgtgattcatttggatcagatgtttgtttgaagagtgtgtgtggaatatttattgcctctttgctgctaggaaaaatatctccctggacggggcgtttctactcgctgctggatccctcttatgctaagaacaacatccccatcattgcttctgtgtctgagcatcagcccacaacctggtcctcatactattttgacctgcagctcctcgtcttcatgtttcca

SEQ ID NO: 64 - DNA, 241 bp, Homo sapiens
  rs2248574 (121)..(121): SNP(121T to C)
tgcgtatgacatgcagaggctgctttgcacaggcctcaggaagcacacatgtgacacgcacagggtgtcttacacaggtctcaggaagcatgtgtgtgacatgcacaggctgctctgctatgcacaggccttgggaaacacacatgtgacatgcacaggctgtcctgcacatgcctgcctctgtcctgctccttgggcttcctggtacctgccagctgccttggttcctcagcctccttgt

SEQ ID NO: 65 - DNA, 241 bp, Homo sapiens
  rs2267437 (121)..(121): SNP(121C to G)
aaagcggggctcagatggcccaaacttcagaccactctcttctccactcggcttttcttccatcctcctggccatgtgtctccccgtgagtcgtggcccaagtctccccacctcggccagccgccaccctctggcctggctcccgccctcctgcccacctggccccttccccctcacccagcatgatggggctgagccgccgggccaggcctttggacaggtcgtacacgtagagcttcac

SEQ ID NO: 66 - DNA, 241 bp, Homo sapiens
  rs2267437 (121)..(121): SNP(121G to C)
aaagcggggctcagatggcccaaacttcagaccactctcttctccactcggcttttcttccatcctcctggccatgtgtctccccgtgagtcgtggcccaagtctccccacctcggccaggcgccaccctctggcctggctcccgccctcctgcccacctggccccttccccctcacccagcatgatggggctgagccgccgggccaggcctttggacaggtcgtacacgtagagcttcac

SEQ ID NO: 67 - DNA, 241 bp, Homo sapiens
  rs2268332 (121)..(121): SNP(121G to T)
ggaaggccagagagagtggcaatgggcagtagatagtagcatggcaaagattcaaaggggcgatgtggataaaaaacaagagatgctctgccggaagtgaaacacacaaagcaacagagagccagccatggggaggaaactggactttgccgtgcataatgtatatttcatattctaataacctgcttaatcctttctcgaataaggtagggtattaaatagtttacgacctcaaaagccc

SEQ ID NO: 68 - DNA, 241 bp, Homo sapiens
  rs2270390 (121)..(121): SNP(121C to T)
aatctcaaacatacacacacccctgattccagaaacacaacaggagctgactcaaaaggtctggaagagttagaaaggggaaagatgacacaagcaatagtgaaggctagtggagggaagcggggctttcgtccaaggaagcaaaaaatcataatgtgttattttttagggagtgtctggtgcaggcatgcagggaggaagaatgactctgagcttgaccattcaacttcgaaactttaac

SEQ ID NO: 69 - DNA, 241 bp, Homo sapiens
  rs2270390 (121)..(121): SNP(121T to C)
aatctcaaacatacacacacccctgattccagaaacacaacaggagctgactcaaaaggtctggaagagttagaaaggggaaagatgacacaagcaatagtgaaggctagtggagggaagtggggctttcgtccaaggaagcaaaaaatcataatgtgttattttttagggagtgtctggtgcaggcatgcagggaggaagaatgactctgagcttgaccattcaacttcgaaactttaac

SEQ ID NO: 70 - DNA, 241 bp, Homo sapiens
  rs227053 (121)..(121): SNP(121T to A)
aaggctgtgctattcccaaggttaaaaatacattcttttttcttttaccgatttcaaattctgttcatacatgttgtcatttgttacagtttgccattggttctgcagtaagaataaatgttaagaaaataagcatgatgtgtaacattttaaaaaaataatacatggtaacatggatgcctcaataatgtcgaaattaaactctaaagttatattatcttgcactttttcatgtaagaaa

SEQ ID NO: 71 - DNA, 241 bp, Homo sapiens
  rs227055 (121)..(121): SNP(121A to G)
gcctattttttcttccctttgaactcaatggttttattctttttatattccagtaatgacagaatatagaatatctggtatcaattttttgtacttgtttgccggcccttagcttgaatcagccaatggtacaagggaaaggcagagcaggatttcaaaagctcacctgagaaaattctctatccagtcctgggaaactcattcaaaatggacctctcctacctctctttccctttcttcc

SEQ ID NO: 72 - DNA, 241 bp, Homo sapiens
  rs227077 (121)..(121): SNP(121T to C)
aggaaagggcaacattgatgaaagcaagtaaagagaaaaatgtaaacaaggaaagataacattataaaagaaagaaataactatgacataaaataggcagtaactaagtgtgagagaaactgagaattgcaaagacaaactagaatgagtaagcagacagagaatcaagaggaagaaagaaaaaaggttcagtgaagaaaaaaatggagtaatgagaggataaacgtgggagaggggattt

SEQ ID NO: 73 - DNA, 241 bp, Homo sapiens
  rs2272615 (121)..(121): SNP(121G to A)
gtgaatttcactttttaagtgatagttgggtattttcagaataagttttgttttcgccttcctgaaaagtctgaagagctttgtactgattgaattctgcgaaaggcaaatttctcgaaagggatacctattattcttccatgagttgctgggaggacagtctaacatttgaaagagaaaacattgaatacaatagttttagaacgatttactgtctgtgataaatactttagaaatacta

SEQ ID NO: 74 - DNA, 241 bp, Homo sapiens
  rs2272981 (121)..(121): SNP(121C to A)
gagggtagttccagagacccaggagcccagaacccgctcgcttccaccgtctcttccctccccacagtctgtgggaactggggaaggacctcccgcttcgcacaggctcagggcagctggcagaacctccacccccacgctggcgtccgtgcttgggggcatcagcctctatccactggccgcccgcgccccctcgccccgctgtctcgcggggtggaaagaccacagctgtgcccccgaa

SEQ ID NO: 75 - DNA, 241 bp, Homo sapiens
  rs2274760 (121)..(121): SNP(121C to G)
atataatattcaatatctgaatcctattcttttgttttactcagactctctctactgaagttccaactggtcttcgtgaccttcttgggtttttgttccctgatctcactaactatgtaacacataaagtaaatgtggtgtgctacagaggttgctaatcagctaaggcatcagtaagctgacaagtgacaacactcgagaggggccacagtacagggttctctccttgaggttcttgcca

SEQ ID NO: 76 - DNA, 241 bp, Homo sapiens
  rs2276015 (121)..(121): SNP(121A to G)
tggtcaccacctgccaggcaccagccctccacaacatttaatttcctcatttactcctcagatcgccctgggaggtctgcagagcccactctggatgaggaggctgaggctctggacaccaagcccggtgctcatctccagcttgagcgcccccaaacgcccaccccccagcggcgcggccctcacatcctgctgctgcttctcctcgtccaggttcaccgtgctccagccgatgttctcc

SEQ ID NO: 77 - DNA, 241 bp, Homo sapiens
  rs2276048 (121)..(121): SNP(121G to A)
aagggtggttggaggtgaccagggtgctgcctactccaaggtcttgtcagcagcctccccacctggcctacaggtgaagctagatgtgaccctgggtgacctgaccaagattgggaagtcgcagaagttcacgctgagcgtggatgtggagggtgggcggctggtgctgctgcggagacagcgggactcccaggaggactggaccaccttcacgcacgaccgcagtgagccagggccagac

SEQ ID NO: 78 - DNA, 241 bp, Homo sapiens
  rs2283264 (121)..(121): SNP(121G to C)
tttagggcctcagaaatcttactagtttaatgccaaactgcttatggacttcactttttagagtagtaggagatcaaatgagagaagtgacagtggccagatcctttgccattgtaaggagtttggatttcctttgagtgagggttttgagtctgaaaagtgatttgcatctccacctacaataactgtcacctttaaacacaccctccgggaggaggagccaagatggccgaataggaac

SEQ ID NO: 79 - DNA, 241 bp, Homo sapiens
  rs2283264 (121)..(121): SNP(121C to G)
tttagggcctcagaaatcttactagtttaatgccaaactgcttatggacttcactttttagagtagtaggagatcaaatgagagaagtgacagtggccagatcctttgccattgtaaggactttggatttcctttgagtgagggttttgagtctgaaaagtgatttgcatctccacctacaataactgtcacctttaaacacaccctccgggaggaggagccaagatggccgaataggaac

SEQ ID NO: 80 - DNA, 241 bp, Homo sapiens
  rs228589 (121)..(121): SNP(121T to A)
gaatcaccgccagtctcaactcgtaagctgggaggcaaaaccccaaagcttccctaccaagggaaaacctttggcctcaaaggtccttctgtccagcatagccgggtccaataaccctccttcccgcgtccgcgcttacccaatacaagccgggctacgtccgagggtaacaacatgatcaaaaccacagcaggaaccacaataaggaacaagactcaggttaaagcaaacacagcgacag

SEQ ID NO: 81 - DNA, 241 bp, Homo sapiens
  rs2287830 (121)..(121): SNP(121T to G)
tgccgcccgcccccaccaccagcacccggcccccggccaccgcctcagccagctcccggggcagcccccgcgacagtgccatggcgggacaaccacgagccgcggcggaggcggaggcggtagaaccgagccgcggccggaagcgggtgggggaagggcggcctggtttccgctcctgcgcactacagcctccggcagacacaggcagggcacatcgcgccggggtgtgctgcagcccgcg

SEQ ID NO: 82 - DNA, 241 bp, Homo sapiens
  rs2288881 (121)..(121): SNP(121A to G)
aagcatgctgtgattgattggtgaggtctgcctcggcttgggcgcgggccctgcagcaggaagccagggtttgtttgccatgacagtgagagcaaatccattgagggctcacatcatggaacatttcttgggagccctgccctgtgccccgtgccttacctcatcagttcctaccagcaactctgtgagactggtcttgttaccctcacctccagtttatagaaggggaaatggagacatg

SEQ ID NO: 83 - DNA, 241 bp, Homo sapiens
  rs2290679 (121)..(121): SNP(121T to C)
aacagggatgcgctgagcgatttcttcgaggtggagtcggagctgggacggtaaggcgcgggctccggctggggaagcccgcggcgtgcactgggggttgtccctctcgcagcgacggcttggagggtgcgggagcctgccttcgtgcccttcgatttctccctacctagttagtgtcttgagagagagctaaccttcattcaggtgcggctcgagtccttcccaccccaccagagcgcct

SEQ ID NO: 84 - DNA, 241 bp, Homo sapiens
  rs2294638 (121)..(121): SNP(121C to G)
gatctcaaagacgaatttctccactgggcggtgctctttatccaaaatcaccaccaccactttctccacatcattctgcaaagagaggaacccccactgcagagccaggcaccccacccccactacaccaccctgcctccacccccttgcccaaggaaggaggagaaacggcccactcacagccctgctcagctgctaggctgggggtccccaagaggccagatagcgagtttgagaagcc

SEQ ID NO: 85 - DNA, 241 bp, Homo sapiens
  rs2304136 (121)..(121): SNP(121G to A)
agtggcccaggccacaggtaagggggactgtggttggagggaccagaaggacaggagctcagccagagaaaatgcagaggtcaggtggatcggagagatggtggagagaggctgtgaaacggggttgaggagaaggctgagagggaggagagcctgatggggacagatttgcaggggtgtgggagagaacacaggggcagcagttcaggtgagggaggaccaggcatgtgctgggacaggg

SEQ ID NO: 86 - DNA, 241 bp, Homo sapiens
  rs2304579 (121)..(121): SNP(121A to G)
cacggttagaggtatgtggttagttgctaattttggaattatatactagattcttctacctagtggaaggtattacaacctttacataaaatataggtttactaccaaggtcaagacccaaattagacttttcagaagtgttgtcaataatcctgtggaaaggctgcagatcattgctaaatatgtctggcagggcctcttcctttgtcaacaattctcttcctactttatcatttttatt

SEQ ID NO: 87 - DNA, 241 bp, Homo sapiens
  rs2304580 (121)..(121): SNP(121T to C)
aagggacaaagatatttcagtaagaggttcctatattctaatcaggctgaagtttattaaactagatttctagtccccaaccatgtggctgattcagtgggtctgaagaaacctatatttttgataagctgtgcaagtgattctggttttcccatagaacaggcaaacacaaaattgcccccgactaggaaaacccctcccattaagggtctcaataatgtccttaggaaaaaacaaggtt

SEQ ID NO: 88 - DNA, 241 bp, Homo sapiens
  rs2304669 (121)..(121): SNP(121C to T)
ccaagagcccatagtcatacctaactccggctgcggcttcttgtctcccacatggacgatggtgctgctgtagctgcactggctggtgagcgacgccacactctctgccttgcccggcagcgccagcgaggtcaggtgcgtacctactcccgtgcggctgttcaccctggagggcggctctgcctcttcatgagaggaagggggaacaaacattagtgtgtcattacaatgcactgtgcag

SEQ ID NO: 89 - DNA, 241 bp, Homo sapiens
  rs2305540 (121)..(121): SNP(121G to A)
cgtcagggccgggtgtgacaacggtcagaggggaagcaggcagggtggagggctgcagctaccaacagacaagtgtggtcgcaggaccgggggttacagcacagggaccaagagggaggagctgcaaggccaagggagaaagcagagaccaggggaacggggccgatccagcaggcgtggacggccagaaacacacgcgggagagaggcaggtacccggggctcccaggctactgtgagct

SEQ ID NO: 90 - DNA, 241 bp, Homo sapiens
  rs243336 (121)..(121): SNP(121C to G)
tcctcttagggactatcccacgcgatcagccagtcgcagcgcagagcaatgagcttgacacctccttcctccaatcgcagggcagctcctcccagccaatgacaggcctccgaggggaagcccagtccctcgcggatatgtcaatgggcgagccctgggaggaatggaagtcacggatctcgcgaggatgcccccggcgcccctcaattcccgaggcgggccgtcacgtgacccgggctcg

SEQ ID NO: 91 - DNA, 241 bp, Homo sapiens
  rs243387 (121)..(121): SNP(121T to C)
gcctggggacaccaacaaatctaagccctccctagctgcttggtaactgtgtcatgaagctgccggacagacacacgtggcatctccctgggcaggagagcaggcctgcagcatgggtcctgttcccgtgtgccgtgggtggcagtggctgcacctggcactagggctgctctgtggatgtgggtgacaacggcaggaggggatgctggccttcctgcacatagacctgcagttagtaaat

SEQ ID NO: 92 - DNA, 241 bp, Homo sapiens
  rs243387 (121)..(121): SNP(121C to T)
gcctggggacaccaacaaatctaagccctccctagctgcttggtaactgtgtcatgaagctgccggacagacacacgtggcatctccctgggcaggagagcaggcctgcagcatgggtcccgttcccgtgtgccgtgggtggcagtggctgcacctggcactagggctgctctgtggatgtgggtgacaacggcaggaggggatgctggccttcctgcacatagacctgcagttagtaaat

SEQ ID NO: 93 - DNA, 241 bp, Homo sapiens
  rs2445837 (121)..(121): SNP(121T to C)
accaggagcagctcctgcggcgcttcggaccccctggacctgaggcctggtgaccttgcaagcatcccatggggcgggggcgggaccagggagaattaataaagttctggacttttgctatatggtgctttgtggtctctgggggacactgtctggtttcataagccctggccttggctgggaccctcctgaggcccgcaccgcccctcatcctcagcaaaagcccccggactcccacctg

SEQ ID NO: 94 - DNA, 241 bp, Homo sapiens
  rs2561829 (121)..(121): SNP(121C to T)
caagatgagatttgggtggggacacagccaaaccacatcaataggtaacatgggaattatgggagctacaattcaagatgagatttgggtggggacatggccaaaccatatttccatcctcgcagaagattctattgaattgcatgtttcaaaatataagcttgttgaggccaaataccttgtccttgctctatttccattacctggaacaatgccaggcatagaccaggtacttagaaaa

SEQ ID NO: 95 - DNA, 241 bp, Homo sapiens
  rs2561829 (121)..(121): SNP(121T to C)
caagatgagatttgggtggggacacagccaaaccacatcaataggtaacatgggaattatgggagctacaattcaagatgagatttgggtggggacatggccaaaccatatttccatccttgcagaagattctattgaattgcatgtttcaaaatataagcttgttgaggccaaataccttgtccttgctctatttccattacctggaacaatgccaggcatagaccaggtacttagaaaa

SEQ ID NO: 96 - DNA, 241 bp, Homo sapiens
  rs2616023 (121)..(121): SNP(121G to A)
ctccttccagtgtctgatatccatcataccttgataatatggtgtaccttagctagcattccaatttaacattccatataatatacactgctcttcccaagtcaaaaaatgtattttccagccactcaataatttggaaggtgtggttctagttgtggatgccatcctgtaagccacctgcagctacacagagtagtctttataatttctaattggcttgtgtcttcaaaactcatgcaat

SEQ ID NO: 97 - DNA, 241 bp, Homo sapiens
  rs263438 (121)..(121): SNP(121C to A)
gcttcttcacgttgaagtccatgatattcatcctaggcggcagccgggcggagccggccgacctagcggcggcgaggcggaggtgccgcggccgggctggggcgcagggctagagcggcgcgggcagacgtggtaggtggagggaggcggcgccagccccgcagccgcagctgtcgtttccgtgaacggggaggatgaaggcggggcgagggaggagaacaagcgcgcggcggcagcgcca

SEQ ID NO: 98 - DNA, 241 bp, Homo sapiens
  rs2705 (121)..(121): SNP(121T to C)
ctccctgggcaggagagcaggcctgcagcatgggtcctgttcccgtgtgccgtgggtggcagtggctgcacctggcactagggctgctctgtggatgtgggtgacaacggcaggaggggatgctggccttcctgcacatagacctgcagttagtaaatcataagcccaaataaacaggttgtttgaatatagtttcttggttctccgttgaaaccgtgggacacagggcaggcccacctgc

SEQ ID NO: 99 - DNA, 241 bp, Homo sapiens
  rs2705 (121)..(121): SNP(121C to T)
ctccctgggcaggagagcaggcctgcagcatgggtcctgttcccgtgtgccgtgggtggcagtggctgcacctggcactagggctgctctgtggatgtgggtgacaacggcaggaggggacgctggccttcctgcacatagacctgcagttagtaaatcataagcccaaataaacaggttgtttgaatatagtttcttggttctccgttgaaaccgtgggacacagggcaggcccacctgc

SEQ ID NO: 100 - DNA, 241 bp, Homo sapiens
  rs2742946 (121)..(121): SNP(121T to C)
agaaaaccaaaagaagtcccgctacagtgacctgcaacgcgcgcacgctcgccgcggcggtcacttatagagcctgcctggaaggcacgcctctttattcaaaatggcccgcctcggccctgcccccgccggccctgggcgttcacagcccgcttaaattgcaaacaggcttcctccggctggtctgacgccgaagaccgcggagccgcggattcaaagaatgaggaagcgctgataccgg

SEQ ID NO: 101 - DNA, 241 bp, Homo sapiens
  rs274867 (121)..(121): SNP(121A to G)
agaaaatagtcacggaaaattccagaaacatggagacatggaggcagagacaccagagacagccagggaaacagaagagccaggttttgtacacgtgacaaagactgggagacagagactaggagactgaactgcagagaagccgcaggataaagaaaaggatccatctattcagtgaataaggggccaggtcccatgctgtgggctggggatattctagttggagcgatggagttgggca

SEQ ID NO: 102 - DNA, 241 bp, Homo sapiens
  rs2750440 (121)..(121): SNP(121T to C)
acctctgcatctcagcctagaatattttgtactctcttgtccattgctgaattcccacttttcctttaagacctagaccaaatgtcatctcccctgtggaaatttagtctcctaaaaatatatatcagcaaaaaggtctggtttgtggagaaaaatgataaaagaagatgtggtgattctgaatctgagttatgccacttgaaatctaggtatgaggaagttactgaaacttttgatcctc

SEQ ID NO: 103 - DNA, 241 bp, Homo sapiens
  rs282065 (121)..(121): SNP(121A to G)
atcaacacacttctcaaaacttatccctgtagttaagcaatgcatgactgtacgttgtttaacttagattgagggctcctggaaacatgggctctaaggaggaggaggagatgctaacccaaaggaaaatcttaatactcttaggaacagcaaagcaggcaatggttgatgggtggtgggtatgcaaccagcaaatgctcattattcatatgtttattcagattgagctctgtgatggtta

SEQ ID NO: 104 - DNA, 241 bp, Homo sapiens
  rs2854455 (121)..(121): SNP(121T to C)
ctctgcaaccccccagcagagtcccaaacatctgacatgtagccaatggaggacatgcaagtggtggggttcccagcagggaccacctcctgccttgggtttccaagacagagcgaggggtgctgctggggcgtggtttgcaggggccttgtcagaactcgatgctttctcctccgtctgggtcctaactgcagtgcatctagagaccctcctctgactcaggcataaggacggcccgcat

SEQ ID NO: 105 - DNA, 241 bp, Homo sapiens
  rs2854461 (121)..(121): SNP(121A to C)
ccctggggaggacagagggagtctccacggctccacacccccatgctttgtcggagatctggccaggcagagtggtgccctcttgactgagcctcgagcctttgctttccccttactccaaataaagccgcagccctggcgagggagcccagaggcctgaggacagcctgctgggtgcctggcactctctagatcctccctgcctgggcggtgggcatctgcaattctccctgtctcgaac

SEQ ID NO: 106 - DNA, 241 bp, Homo sapiens
  rs2866635 (121)..(121): SNP(121G to A)
cacagcaggtgacatttattgaagatttactatatgccaggctctctattatatgctctctatccaattttttcatctgactttccaaattgttcatcaagaaatattcattgaacaactgttatgtgccagatacggttctaaggtcaggaagcacaacagtaaatagaatagacacagtacctgttgttgtagagttgctattctcttagaggagatgggtaatacagaaaaatctcag

SEQ ID NO: 107 - DNA, 241 bp, Homo sapiens
  rs2910199 (121)..(121): SNP(121A to G)
gcttttacagggcagtagatgaagaaacagtgtcagagactagcgtgaaagtgaatagagtctatgaatcactcaggctttgtttttttccacttaaaaatattcctgaaatttgcttagaagtgttaaggcctaattagccaaaaccaccaggagagcgcatctcgaagaggaatcagaaagcgcaggacaagacagttgaatcttttctaagacttgaggtccctctggagggttctga

SEQ ID NO: 108 - DNA, 241 bp, Homo sapiens
  rs3087386 (121)..(121): SNP(121A to G)
gaattagttctgtgtggattccgcaaagcatctgtgtttctggtgctttgctgcaactgctgcagagtgcagtctctgaaatcagtgctgctcttctcagccttatcctcctcctgggaaaaggctggagaaagcctgctggcaacactgttgaccatgggcaccaagcaatcctgtgtctttaaggcaccattagagctaggagtgtgtccattaaaaatggcagtgctccctctgggat

SEQ ID NO: 109 - DNA, 241 bp, Homo sapiens
  rs3131687 (121)..(121): SNP(121A to G)
gggcctcgggggcgattccgggtaggtaaagcggaacgagggcgtggcctctcttgtgagcccgccctccccgtgtcgctcccgccttgagggcggggctctttcccaagtgccctctgtaagagccgggccgctctctccggggcgtggcgaagaggggcggagtcacgagcggggcggtgagacttcctgcccagtcgcgggccagcctagcgcttcagccggcggctcatttccggtg

SEQ ID NO: 110 - DNA, 241 bp, Homo sapiens
  rs3136820 (121)..(121): SNP(121G to T)
atgcctaatgcctgaactcttcaaaaccaattgctaattctctatctctgccccacctcttgattgctttcccttttcttatagttttttatgctaattctgtttcatttctataggcgaggaggagcatgatcaggaaggccgggtgattgtggctgaatttgactcgtttgtgctggtaacagcatatgtacctaatgcaggccgaggtctggtacgactggagtaccggcagcgctgg

SEQ ID NO: 111 - DNA, 241 bp, Homo sapiens
  rs3733995 (121)..(121): SNP(121T to C)
agctactgctacctgaagctgtgcctacctacagtggcccctgaggacttaaacaatttttttctcacatatatttagtggtactgctgggaaatggaataagatctggaagtttgtaagttgagggcaagcccaggtcatcctaaaaacctcgctaagtttatggttctttattatttcaggtcagaaaattaattgttttggatccaaagaaacggctgactacatttcaagctctcca

SEQ ID NO: 112 - DNA, 241 bp, Homo sapiens
  rs3735048 (121)..(121): SNP(121G to C)
tctgtctctattgccaaaatcaaataaatggcttctcagctgctgtttacaacaaacttccctaaccaaaacagaacctctaatttagggctaggaatagaagtcttttgatcccagtccgtgggacagttttgtatgctgtatcttgtacacaggttgtaggttggtttattgccattttgttttatttctgctgtataaaaaccaagagccagacaattagacaactatggaggaatga

SEQ ID NO: 113 - DNA, 241 bp, Homo sapiens
  rs373759 (121)..(121): SNP(121T to C)
gagaaaaacaaaagataattgaaatagcaagggaaatcataaagtacgtgagtctgatagcattggctttagattggaggcatgttagcttttctgctgagagagaaggaaaagtgatgctaataggaaagtcttaagctggagaggaaggagtatgagggaattatatttgctattgtgtttttgctgagaaatagaaagcatgctcatttgctaagaattggagggatggtaactggcc

SEQ ID NO: 114 - DNA, 241 bp, Homo sapiens
  rs3741049 (121)..(121): SNP(121G to A)
ccaatactgtttgatttttcttactctatgtactttacaaactgaactgaaagatgggctctataaatgttgattttagccgtgtacttttggagaatatttttgtatgcctattgcatcggcatggctcagtcattaaagaaattttcccacatttaaatagggtaatttctgacgcttagctggcaactgcttggctaattcaagtatgtgaagataagacatgagtggctgggtgcgg

SEQ ID NO: 115 - DNA, 241 bp, Homo sapiens
  rs3742557 (121)..(121): SNP(121A to G)
gtttgagcaaaggagcttaaaatgatctaaggctagcatgattaatagtttccaaaaaggatgaacatgagtacttttatgagcaatgctagataaggtatccatgtttttctttcccctaaagaatgttgttttagaatatttttgccatcaaaatttctgatttacaatcatgatatacaactgagagatgtacatttatggatgttaaatgacataaataagtaaactagacacatac

SEQ ID NO: 116 - DNA, 241 bp, Homo sapiens
  rs3744357 (121)..(121): SNP(121T to C)
gtattaggaataagatactttttattccatcctctatggagacaaaaagctgctcctcctttgagtactgacctacatagatttccatttctctcttcagcctcaggccaaacaaacaactggacagggcagcagaacaaggctgtctcctcaggatcctgccaaaggcactgtcctctccaacctggacccagccccacttgcccttattggttgcttacacccagagtgcacactcaca

SEQ ID NO: 117 - DNA, 241 bp, Homo sapiens
  rs3750496 (121)..(121): SNP(121A to G)
agggtgcagagttccagggtaagaggcccggtctctaaccttccagctcactgctcttttctgctataccagggcaccgctgtgcagtcatccaggctgccggggactagcttccccgacatggcaacaaaaactacagaactcgctcacatcttaaacatggccaaccttgactctgccaagatataaggtttagaatgagttgctaacattcaatttacagaattggatttgaactgga

SEQ ID NO: 118 - DNA, 241 bp, Homo sapiens
  rs3781868 (121)..(121): SNP(121G to T)
aacatgtaaagatagagaagtagaaaggcattcctcattccaggaggacagactgacatcaacaaaggcaaggaagtagaaaatataatgtacattccatttaaaatcaaatacaacagcgtctttttctcttaatgccaaaaattgagaattagagagttgaagacttcaattagcgtagaaataggaagaaccctagattaaaatgccttaaagattttatatgcttatagcaaatcta

SEQ ID NO: 119 - DNA, 241 bp, Homo sapiens
  rs3786738 (121)..(121): SNP(121T to C)
tgattcttaaccaagctccttagctgatcctcattgcacctccagatgtgaggcatgatttctttttatttaatctgcctcaatggccttccaggcaacaacagctaggaaatggcatgattgccatctcagaaggtgctctcttttttctcccagtggtctgttttgagacatgagttcggaaacttacactgtgtggagcctgacaatgaagccaacaatatggaaggcgggacgaaga

SEQ ID NO: 120 - DNA, 241 bp, Homo sapiens
  rs3791213 (121)..(121): SNP(121T to C)
aaagagcttcttcaagaacttgattgttatgtctcagtttgcaaagtgaatggaatgtctaaacacatacaagtcttggtgttgattccaagtacagcagcttatttgagagtctgagactagagcagtagtcagttgattaagtctagggtgttattgggaccacccttcaggtaatccagcctacagtttcccctcaggcccatgcttttcatcttagtaggtgtaggctgctaggcaa

SEQ ID NO: 121 - DNA, 241 bp, Homo sapiens
  rs3803798 (121)..(121): SNP(121G to C)
cctgttgtccccaccccaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctggtctgttggccctgcggccagggtcctccctgcggatccgcaccctcccctgggcccatctcaaggctgcccccttcctc

SEQ ID NO: 122 - DNA, 241 bp, Homo sapiens
  rs3806201 (121)..(121): SNP(121A to C)
ccctgcgcccagtctccgcaagtcctcccacaacaatgtccagactaattgccttcccacgtcacaaacggggaaaccgaggtatgcacgcccagagacaggcggagcgtggcctcgatcaccaccgagagctgacggccgcccggattcccgccctcagagaatcctggcccccagtcgttccaagaccctttacggactgcagcttaaaggaaccggcctctgccattgagaccaaact

SEQ ID NO: 123 - DNA, 241 bp, Homo sapiens
  rs3806202 (121)..(121): SNP(121C to T)
cgactgccagccgggctggggctgacccaccgcgccctcactgacccccacgccctgcgcccagtctccgcaagtcctcccacaacaatgtccagactaattgccttcccacgtcacaaacggggaaaccgaggtatgcacgcccagagacaggcggagcgtggcctcgatcaccaccgagagctgacggccgcccggattcccgccctcagagaatcctggcccccagtcgttccaagac

SEQ ID NO: 124 - DNA, 241 bp, Homo sapiens
  rs3806204 (121)..(121): SNP(121G to A)
cgaagcctcccctgcgcgcggcagccaccgtgcgtaacctcccctgaaccgagtagcagccgcctgagcccacccagccctgggcgaggctgcctcagaagccggatttcagcagagtgggtgcaggatctgctttaattctgctcatcttattctcggggaccacaccctgcgcgccgggaggaagggctccaaaaccacagggcggaggcttccgcgcggccccagacttgaccatcta

SEQ ID NO: 125 - DNA, 241 bp, Homo sapiens
  rs3806207 (121)..(121): SNP(121T to G)
gactggacattccttagggcaggggttgtaacttaagtcgtgtgtgcattctttataatgaagcattttacaaagtggtttcacatacattactgcttccgtttctcattacagccccattaatattccccacctgatgaggaaactttcttcattctccttcctgcttcctcttctctctcctcttcctctagaatgcgtgtatggtctatgttccttctgtctctaatccagaaagaga

SEQ ID NO: 126 - DNA, 241 bp, Homo sapiens
  rs3809454 (121)..(121): SNP(121C to A)
ccaccgacccagccgccccagactttgccctggcctatcccgcgccgctcctcaaactagacccgccccgacgtcgccctggcctatcccacgccgatcctcaaacgaggcccgcccccgctgcacaagctccgccctcgttgcaccgtcctgaccctcggcctcgcccccgccccctggctcttcccgcgcagccgaatgccgcgacctggctcccacccttcgtcgcttcgcgccgcat

SEQ ID NO: 127 - DNA, 241 bp, Homo sapiens
  rs3825609 (121)..(121): SNP(121G to C)
atatcattctgaggtattaattcagaaccatcttccaagctatactggtagcaccacagacttactagctgctggtcatactgcacatcaatgattggtttgagcaaaggagcttaaaatgatctaaggctagcatgattaatagtttccaaaaaggatgaacatgagtacttttatgagcaatgctagataaggtatccatgtttttctttcccctgaagaatgttgttttagaatattt

SEQ ID NO: 128 - DNA, 241 bp, Homo sapiens
  rs4543783 (121)..(121): SNP(121G to C)
ccccaagattcatatgttgatactgaatcaccaatgtaatagtattaagaggtggggcctttaggagatgactaataagggccgagccctcatggatggcattaaggcccttataaaagggcttgagttggtgggttccatctcttctgccatgtgagaaggtcctcacctgatgccaaacaccagtgctttgatcttggacttccaagcctctagaactgtgagaaatgtctactattta

SEQ ID NO: 129 - DNA, 241 bp, Homo sapiens
  rs458486 (121)..(121): SNP(121T to C)
ttttgagttgtgagttacaaaacttgactcactgtcttcagtctcataatttaccttgcgtttggctctaagtgtgtatttatttccatatagtccaaaggactgctcagtttctaacgttccatctccaaagtgacagtctataaaaccatctgtgggtgttttattttcaaaagctccacgagtggttttagttaaatcactagccaatgcattatccttctcaggattactattacaa

SEQ ID NO: 130 - DNA, 241 bp, Homo sapiens
  rs4699052 (121)..(121): SNP(121C to T)
gagatgcaaataaaggaaataaagaagcaatattcaaagagatcatttctgaggaattttcaaagtttgagaaagatgtgacttcttagattgaaagtgtaccccaaaaagcatgtaggaccgttaaaggtaactccatacctagtcacaaatttgtgaaactagagaacatgaagaataaagagaaaagtcttaaaatcttccagaaagaaaaagagaaaagattacctaaaaggcctga

SEQ ID NO: 131 - DNA, 241 bp, Homo sapiens
  rs4699053 (121)..(121): SNP(121C to T)
ttcaggtaataatagaaaatccaaggtgactagggactggagcggaaccccagcatactgcagcagccatatgggaaaatggccagactgttttccgtacatgtcctggtgcttacttctcttcactgggcagagctgcttgacctgagacaccaccacagccaccctgccattgcctgaccacttcaatcagaggcagcccagcatttctgtaaggagaaaatcctaaagtcaacccaca

SEQ ID NO: 132 - DNA, 241 bp, Homo sapiens
  rs4818 (121)..(121): SNP(121G to C)
aggcaagatcgtggacgccgtgattcaggagcaccagccctccgtgctgctggagctgggggcctactgtggctactcagctgtgcgcatggcccgcctgctgtcaccaggggcgaggctgatcaccatcgagatcaaccccgactgtgccgccatcacccagcggatggtggatttcgctggcgtgaaggacaaggtgtgcatgcctgacccgttgtcagacctggaaaaagggccggct

SEQ ID NO: 133 - DNA, 241 bp, Homo sapiens
  rs4818 (121)..(121): SNP(121C to G)
aggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtgtgcatgcctgacccgttgtcagacctggaaaaagggccggct

SEQ ID NO: 134 - DNA, 241 bp, Homo sapiens
  rs491071 (121)..(121): SNP(121C to G)
tgttttggctataggagtctttgtcatcatcttattatctctctcaatgcatagttaaacattttttctcccattctataattaatatatcctgctcgttttgtctatctctttctgcttctttttcttttcatgtctctgcccttatccatttttttctttgtccttctttttgcagttcattttgcaaaaatggaattattatttacattttaaaattactgtttcccatttaatattt

SEQ ID NO: 135 - DNA, 241 bp, Homo sapiens
  rs491528 (121)..(121): SNP(121G to T)
tagtgtgtgttgagaacatagcagaaaccacttccttggtttctcctttgtggaaaaagtaatagaggtaaaatcgttttggatgagtcatgtttaatctttggaagttttagaagaaatggaattctgtaatgttaaaactcttttcctttttagagttcaacttgctgtgaatagagtaactgaagaagcagtcgcagtgaagattgtagatatgaagcgtgccgtagactgtccagaa

SEQ ID NO: 136 - DNA, 241 bp, Homo sapiens
  rs4983545 (121)..(121): SNP(121C to T)
aggggcctgggggcataggctgggttatgagattcctgtgactccagggatgcaagcaacttttctagacagtaccaagaacagccctcagtgacttgagcggcaggaaagccatgcaaccgcacggctctgccccaggccacgggcgcttccttcctcaggccggagtccctcagcagcactagccaccttgagatggggtgggacacacaggcagaagccagcctttgagtattcccag

SEQ ID NO: 137 - DNA, 241 bp, Homo sapiens
  rs4983548 (121)..(121): SNP(121C to T)
tgtggcctgggggacagccccactccataagctgcccgatgttctgtatctgtccacgatggtgatgagaccctaggagagttgcaagtctgaacacagggaaatctttagaaatccccacgatccataagattgaaaacaatcctcctctctgagaacttcatctatttctgggataaaccaaatctgtgtttagtcacacgtgactccaactgagtaactttcaagtcatttccctaag

SEQ ID NO: 138 - DNA, 241 bp, Homo sapiens
  rs518116 (121)..(121): SNP(121A to G)
agcagccagagtatgtgtgtgcatgtacacgtgcatgcgtgtgtgtgtgtgtgtgcgcgtgtgtgtatgtgtaaggtgagagaggcagaaggagggtggaaagaatggcgtttttggtgcagtgcaaactaatttgcccaaactgaaaaagctggtgctctcaccattatgccaggctacctccctgtgtagtagatgacacagctgatgtagatgtataaaaatgtagcccctacagttt

SEQ ID NO: 139 - DNA, 241 bp, Homo sapiens
  rs518116 (121)..(121): SNP(121G to A)
agcagccagagtatgtgtgtgcatgtacacgtgcatgcgtgtgtgtgtgtgtgtgcgcgtgtgtgtatgtgtaaggtgagagaggcagaaggagggtggaaagaatggcgtttttggtgcggtgcaaactaatttgcccaaactgaaaaagctggtgctctcaccattatgccaggctacctccctgtgtagtagatgacacagctgatgtagatgtataaaaatgtagcccctacagttt

SEQ ID NO: 140 - DNA, 241 bp, Homo sapiens
  rs565435 (121)..(121): SNP(121C to G)
ttatctgtgatgctcagtgtctccatgcaggatggtcaagacctccacttgtccattatcccttctctttctcttctatttgcttcctctgggaacttgccccccatcagttgcccactcctttcttatattgtcagtccgtttctatggactccttttttggtttacaaggatgctcaggttaccccaaatttaaaatactttctcttaatcgtgctactcccctagcactatccttcct

SEQ ID NO: 141 - DNA, 241 bp, Homo sapiens
  rs573890 (121)..(121): SNP(121G to C)
caaccagatgtataaggtgactttggcactgagggccagccaacatgctcttggccaccctagtgcttgtgcaattagctcatctacagaagagtcatgatggcatggatggaagccaagggcccaaaatcatgggccttttctcagaaaaactaatctagctgcttccactgctgaatgacacttggccttgatatgacaccatcatttgaaaagaccaatcagttctttggtggcaaca

SEQ ID NO: 142 - DNA, 241 bp, Homo sapiens
  rs5745095 (121)..(121): SNP(121T to C)
gttgagccgctgcttgggaaccagggccagagcttcccgtttctgacccctccaccactactcaaatgcccgcagctagcctcacagtgaacccaggctgactggcgcccacggctgcactgggcgacccctggtcctgctaggtggccttcctgggggacgagcggctgtcccctggccccatcactcctcattcctgagggttctccagattcgcttccaggaatgaagcctagtcagc

SEQ ID NO: 143 - DNA, 241 bp, Homo sapiens
  rs5938 (121)..(121): SNP(121C to A)
tcctcatcatccacatcttcatcgtgttcagcttcttcctggtcttcctcatcatcctcttctgcaacctggtcatcatccgtaccttgctcatgcagccggtgcagcagcagcgcaacgctgaagtcaagcgccgggcgctgtggatggtgtgcacggtcttggcggtgttcatcatctgcttcgtgccccaccacgtggtgcagctgccctggacccttgctgagctgggcttccagga

SEQ ID NO: 144 - DNA, 241 bp, Homo sapiens
  rs609557 (121)..(121): SNP(121T to G)
aaggtaatacaagggggctggaatagccaaaataatcttgaaaatgaagaaaaagttgaagcacttacacttaccaatttcaaaacttactgtaaagctacagtaatccagacgatgtactggcatgaggttagacacagaaattagtggaacaaaataaaaaatccagacctttcttatttatctagtcaactctgaagcatggaatccagacatttatggtcagtttattttttttcag

SEQ ID NO: 145 - DNA, 241 bp, Homo sapiens
  rs615942 (121)..(121): SNP(121T to G)
gtgctcttggttcggatattggtcagtaggattctgacgtccaagtgcctgtggacgtcggcgccagcatagaggtgcgtgatgaaatcaagaacctcaaacgtggcctgcacggggctgtactggggctgagccgggccctccaggctcatgcccggctgcaggtgaacatagagtgtgtgattcaccagccgggccgccgaggtcaggatggaggccaggcgaggggctagggaggcca

SEQ ID NO: 146 - DNA, 241 bp, Homo sapiens
  rs625120 (121)..(121): SNP(121G to A)
tggttgagatctaaaaaggaacactgaattcgtcaattcagaggctcgtaggaagtaggaaatccttatcttttcgtttgaaccttgagtggaacctagatattctggattaatgtataagtgaagttcatctttatatggtgacactcatactctagttgactacctaatagttcctctgccttctttcagttccgccaacatactgtgttctttaacattgtagaaccctttggctatg

SEQ ID NO: 147 - DNA, 241 bp, Homo sapiens
  rs6267 (121)..(121): SNP(121G to T)
ggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggtggggtccgggccagcaggtgctcagctctgggacagggaccca

SEQ ID NO: 148 - DNA, 241 bp, Homo sapiens
  rs632758 (121)..(121): SNP(121T to G)
caaccaagaagataaggctgactttgttccccttaccccagaccccaactatcccatggcaatcaccacaccaaggccgagccatgggtgggtaggaggcactggcctcttcacagcccctcttggctccatcccctccagtgctttccctagccatactcttttgtagaacgatggctttgctgagcttttgggagccaatggagaagtcctgctgctggcaagtggggacgatggtctg

SEQ ID NO: 149 - DNA, 241 bp, Homo sapiens
  rs641605 (121)..(121): SNP(121T to C)
actcttactgattaattctaagatcatgtgagcttccttggctgtcagaaccaacacttaactgacattttacttgctatgatttgaggaaaaaaacaacccccaaaaatatttttttcttctgatgagccaaatagttcccatctttaacttgtacaattaatattgtgaagagttgatttttcggaaccaaaatgcaggatcctggcaagcttttgtaaggaaatgggctttacacata

SEQ ID NO: 150 - DNA, 241 bp, Homo sapiens
  rs645485 (121)..(121): SNP(121G to A)
gttatgtagcagacataagaatgaagacttggagggctagattaaacttaaataattaaaaaccattcaagtcctttcctacagccatttgaggggaaacctaatgtaaaataagaagccgacatcatctctagtttaggtgtttagatttagtgaccacgggatggagaaatcacctggttaaagtcagcttgaagctctcgtgatggtttttgtttctaattagaaaaataggaataag

SEQ ID NO: 151 - DNA, 241 bp, Homo sapiens
  rs651646 (121)..(121): SNP(121A to T)
agcctttaaagcctgggggtgagggagccctcctggtcaaccctctctaccctagcctcagggagcttcagggccccagcattgaaggaacagggtctgacctcatttgccaccgtagggatggggagactgaggcaggaggtgaatgggctcccagcttggagccctttcccctcaggacttggtttccctaccctagctccgcctgcagggacagaaagacatggtctggaaatggatg

SEQ ID NO: 152 - DNA, 241 bp, Homo sapiens
  rs652541 (121)..(121): SNP(121G to A)
tggtttccattagggtttaagaaaaatcagaaatttatatctcctttttccctgctcaggttcagaactaattagtcagacaaatggagatcaaattgtcagcatcattactagaggaacgtggcttaggaatgagggccagactagtttactgctggtgctacctcagtacctctctgctgtcttaactttgggacagctcacctgaatagggtttgggcctgcagagcaaacacatgta

SEQ ID NO: 153 - DNA, 241 bp, Homo sapiens
  rs664677 (121)..(121): SNP(121T to C)
aaaatgaattcttttacactaatttcttttagcttgaatttttggcaaggtgagtatgttggcatattccacataatgacaaataagtttagcacagaaagacatattggaagtaacttataataacctttcagtgagttttctgagtgcttttatcagaatgattatttaactttggaaaacttacttgatttcaggcatctaacaaaggagaggaaatatatattctctgtaagaatgg

SEQ ID NO: 154 - DNA, 241 bp, Homo sapiens
  rs664982 (121)..(121): SNP(121T to C)
catttcaaacgtctaatgaaagcccactctgccaagtattatgctattttgagatacagatatgtagattattaagcataggctcagcatactacacatgagagtatacagataaagatatgttgacaacattggtgtgtaacaaaatccgtatttataatgtgtttgactctagatgctgtgagaaaaccatggaagtgatgagaaactctcaggaaactctgttaaccattgtagaggt

SEQ ID NO: 155 - DNA, 241 bp, Homo sapiens
  rs704227 (121)..(121): SNP(121T to C)
tgtaaggatgataaagatgtctttctaccctcgagagctctaagtgtggtcaggagagaaacacatgcaaagattatgctggctggaaggaaatgctggaatgaaggtggaaggtacatctagaatgttctgggggtgaggaatagcgaatgatgggttctgacttagaacatgaagggaaggtattaagaaagaggtgatattaagcatttattgagcatctctactttttttgtgtatg

SEQ ID NO: 156 - DNA, 241 bp, Homo sapiens
  rs704227 (121)..(121): SNP(121C to T)
tgtaaggatgataaagatgtctttctaccctcgagagctctaagtgtggtcaggagagaaacacatgcaaagattatgctggctggaaggaaatgctggaatgaaggtggaaggtacatccagaatgttctgggggtgaggaatagcgaatgatgggttctgacttagaacatgaagggaaggtattaagaaagaggtgatattaagcatttattgagcatctctactttttttgtgtatg

SEQ ID NO: 157 - DNA, 241 bp, Homo sapiens
  rs73234 (121)..(121): SNP(121G to C)
aatctcacacagcccggccacaagatttatcaggaagccagttagcagcttaagcctggaaataatgtcacccaaacccccagcacaccaagggaacttctgagagcagctctcgctagcggcattctttcaccctccttagtcccccagttctagctgcgcacggatgccaggaggctcttgagggagtgggtgtcacacaggcagaaaggagggaaccagggagatgggaccatgacgg

SEQ ID NO: 158 - DNA, 241 bp, Homo sapiens
  rs73234 (121)..(121): SNP(121C to G)
aatctcacacagcccggccacaagatttatcaggaagccagttagcagcttaagcctggaaataatgtcacccaaacccccagcacaccaagggaacttctgagagcagctctcgctagccgcattctttcaccctccttagtcccccagttctagctgcgcacggatgccaggaggctcttgagggagtgggtgtcacacaggcagaaaggagggaaccagggagatgggaccatgacgg

SEQ ID NO: 159 - DNA, 241 bp, Homo sapiens
  rs740059 (121)..(121): SNP(121G to A)
tctcacagcctcccgcagcggcgggcggtcagcgccggcctcatgcagctcctcccgccgcctatccgcaccggaacaaagtaagggccgcggaggctcgtacttcacgagcagtcaggcgcgagtacgaggcaggcgacgaagcttctggtcgacggggtcgtggggggagaatgaaaagcgtgcagaatgcaagagctgaccggggaatgtgggtcagagagaggcgggctgtggtgcg

SEQ ID NO: 160 - DNA, 241 bp, Homo sapiens
  rs750621 (121)..(121): SNP(121G to T)
ggagaaggaagagtgctatgtcctgtaatgtgatggagcgataagttatcatcaaggagaaaaaggccggggcctggagcgggacagttctaagggtgctgtttgaattggtcagggaaggtctctttagcaaatgatggtgaagcagaggagatggaacagaggcaagggaccctcattctgtcatcagagggaagagaattctaggcagaaggaacagccagtgcaaaggcccagaggc

SEQ ID NO: 161 - DNA, 241 bp, Homo sapiens
  rs752593 (121)..(121): SNP(121A to G)
cccaccagtgcagaccattcttgccccctgagattctgcccacctcatttggcacctcttctaagaatgcttttctgctctccccagctcgcgggagctctcctttccttaaaccctcataacactgtggccctggccacattctgcattgcattgtgctgctttgatctactctgtacctcctaccttatctccttgactagactttaagctcttttaaggcaagatctgtgtcttgttc

SEQ ID NO: 162 - DNA, 241 bp, Homo sapiens
  rs767298 (121)..(121): SNP(121T to G)
atatgtgtgtctttggtttttgttacttgttttggtcatagcttttgcttaagtcttgttttcctagagaaacttgaggtgtatgtaagtttcgatttatatttttataattagttaatataactaatcttttttttcctcaagaggcacaaagaacaaggaagttaaaaaccaaggatgcgttctaaagacttcattttctcctagcctcctacctctaaccagattttctcagttgagt

SEQ ID NO: 163 - DNA, 241 bp, Homo sapiens
  rs791040 (121)..(121): SNP(121A to G)
gcagtcccctgtgttcttgtgcatctacaggatgacgcattgcttggcaggttcttattcatctttccaggaactcagagaggtctctgggaaatatgggaggcaaacagtttgaaaagcaaaagtgctatggtcatattacttctacagaaagcagctttacagtggcaaggtgaagcccaggttcctgggaggggcccaaactggacaatggtaaaatgagcagtgagcagcatgtggc

SEQ ID NO: 164 - DNA, 241 bp, Homo sapiens
  rs791040 (121)..(121): SNP(121G to A)
gcagtcccctgtgttcttgtgcatctacaggatgacgcattgcttggcaggttcttattcatctttccaggaactcagagaggtctctgggaaatatgggaggcaaacagtttgaaaagcgaaagtgctatggtcatattacttctacagaaagcagctttacagtggcaaggtgaagcccaggttcctgggaggggcccaaactggacaatggtaaaatgagcagtgagcagcatgtggc

SEQ ID NO: 165 - DNA, 241 bp, Homo sapiens
  rs791041 (121)..(121): SNP(121A to T)
cataaacatgcacacactcttcctttggccatggcaaacaaagaacatcctttagtttcactctgtctccaccttgcctgcagcatctttctccaccagcaaggagttccacatgtcaagactttagtgtcggctgcctgcaaatcctgtaaaacgggaagtgcaggggagagatttctgtactcagaggtcagcctagagctctgccatgacctcacagccatagtaagctacatgaaac

SEQ ID NO: 166 - DNA, 241 bp, Homo sapiens
  rs791041 (121)..(121): SNP(121T to A)
cataaacatgcacacactcttcctttggccatggcaaacaaagaacatcctttagtttcactctgtctccaccttgcctgcagcatctttctccaccagcaaggagttccacatgtcaagtctttagtgtcggctgcctgcaaatcctgtaaaacgggaagtgcaggggagagatttctgtactcagaggtcagcctagagctctgccatgacctcacagccatagtaagctacatgaaac

SEQ ID NO: 167 - DNA, 241 bp, Homo sapiens
  rs818707 (121)..(121): SNP(121C to T)
gctgtattcccaggacccagcactgaacctaggaacagagcagagattctcaaaaagtatctcctgaataaaaagtgtttgtggtggtggcacctctagcagtcaggaactccctctttgcctctgagttttgatcctcaggcagtatttatgctggttaatgagtaattcttctctgggtttatggttcagagtagaataggagacctcaggcagcctggcaacctctggcctcgtggga

SEQ ID NO: 168 - DNA, 241 bp, Homo sapiens
  rs871027 (121)..(121): SNP(121A to G)
accacgactgctctgagtaccaggaactcctttttgggatgtcacagatcaccaacttccttctctgtgctgtgtcaatagggacgtcacccctgtggggaaaggaatttgcctgaacaaaacactaaaaagataaaataggctgaagcggagaagaataaaagattgtgctggcatgagctagttttgcaaaacccatttaaataaactcaggatattaccgaccccatgcacacattct

SEQ ID NO: 169 - DNA, 241 bp, Homo sapiens
  rs875382 (121)..(121): SNP(121C to T)
agcgggccagtgggcagttagctgtggcccccgggggcagtgggaaggtagcagaggacctgcctggaacagtggttctttgaccctcccagagcagagttagaactcacagcaaaaactcgagtacgttgaggctcccaatgtcaggaagcagtttctgtggcacttctgtgagctcagggggcctgggctcggtgacccaggagaacaggcccctcttctgtgaagccgttgtgctcat

SEQ ID NO: 170 - DNA, 241 bp, Homo sapiens
  rs9110 (121)..(121): SNP(121A to G)
gaggactagagaggatcgagtgggctggtgcacctagctctgtgatctcctcacctaacatgagcccacacagctaagaaagcactagaagccctgtggtccatacctgttggtggagcaacacctgtttcaggcgttccaccttatccatccgagacaccacggcatgattcggggccatggcaagatggcagcttctagcctcagtcacaggcttccgtttgccatcgaggcacagcag

SEQ ID NO: 171 - DNA, 241 bp, Homo sapiens
  rs9226 (121)..(121): SNP(121T to A)
gctatcaaaatcacagtctagctggtcactggtgattggccctgttaggacagccaaggtgaactcaaggccatgaagcgtgtggctgaagggagtgaagaacaggaaggccaggtggacttcagggccctagtagaccaacaggcccacagtgccctggaagctgaggctcatctatgccaagcagcaacttcgggcactatccttcgggtagtagagcctggggagaaaggaaagttag

SEQ ID NO: 172 - DNA, 241 bp, Homo sapiens
  rs934945 (121)..(121): SNP(121C to T)
ggaaacagccatgaagatctttcatctcttccaagcaccacctggtgtacctcgctggctgccgggctgaggtggggcaggggttacgtctgctcttcgatcctgtgattcaagggggatccattttcgtcttctttggtgtccgacacttcgctgagtcccagagaaggaatatcttcctcatatggtatgcaaatattacctttttccttgttttcacagtaaacacattccttaaaag

SEQ ID NO: 173 - DNA, 241 bp, Homo sapiens
  rs972800 (121)..(121): SNP(121G to T)
atctgtactgtccaatccagaggccactggccacacatggctacagaatgcttaatgaggccagtatgactgaggaactgaatttgtcatttaatttatcgaaatgtaaacaacctaatggagctagtggcttctggattggacagcgcagctctgcacatatacaggtcaatttgcatgtctgtatgtttaactttaatttttaactttatttcctctttctgccccgccaccccccacc

SEQ ID NO: 174 - DNA, 20 bp, Artificial sequence
  Forward primer
aagataggagcaaggtcatg

SEQ ID NO: 175 - DNA, 20 bp, Artificial sequence
  Reverse primer
tacatgccctctacaaaatg

SEQ ID NO: 176 - DNA, 21 bp, Artificial sequence
  Forward primer
gcactgatgaaaagcaaatgc

SEQ ID NO: 177 - DNA, 20 bp, Artificial sequence
  Reverse primer
agtaatgatatcaccaggtc

SEQ ID NO: 178 - DNA, 20 bp, Artificial sequence
  Forward primer
cgatgatgggacaagtgatg

SEQ ID NO: 179 - DNA, 20 bp, Artificial sequence
  Reverse primer
cgagtttggagccatgtatg

SEQ ID NO: 180 - DNA, 20 bp, Artificial sequence
  Forward primer
agagaaggtaaggaggagag

SEQ ID NO: 181 - DNA, 20 bp, Artificial sequence
  Reverse primer
actgagctcacctgaacttg

SEQ ID NO: 182 - DNA, 20 bp, Artificial sequence
  Forward primer
ccagctagactgtgattttg

SEQ ID NO: 183 - DNA, 20 bp, Artificial sequence
  Reverse primer
cctgcaggacaaatttcacc

SEQ ID NO: 184 - DNA, 20 bp, Artificial sequence
  Forward primer
gaccatcaccaagatcaacc

SEQ ID NO: 185 - DNA, 20 bp, Artificial sequence
  Reverse primer
atcagggttgccagggaaag

SEQ ID NO: 186 - DNA, 20 bp, Artificial sequence
  Forward primer
catagaacgttcatcaaggc

SEQ ID NO: 187 - DNA, 20 bp, Artificial sequence
  Reverse primer
cttgtgtgtgccatctttcc

SEQ ID NO: 188 - DNA, 20 bp, Artificial sequence
  Forward primer
tccaagaggaagccctaatc

SEQ ID NO: 189 - DNA, 20 bp, Artificial sequence
  Reverse primer
aaggcagaagatgtagagcg

SEQ ID NO: 190 - DNA, 20 bp, Artificial sequence
  Forward primer
ctatcacctactgccttctc

SEQ ID NO: 191 - DNA, 20 bp, Artificial sequence
  Reverse primer
catttcctgagatgggaagc

SEQ ID NO: 192 - DNA, 20 bp, Artificial sequence
  Forward primer
gttagttgtctcctttgggc

SEQ ID NO: 193 - DNA, 20 bp, Artificial sequence
  Reverse primer
tagaaaagggagtctgttcc

SEQ ID NO: 194 - DNA, 20 bp, Artificial sequence
  Forward primer
tgcttggctatttctgcatc

SEQ ID NO: 195 - DNA, 20 bp, Artificial sequence
  Reverse primer
aagatgatagcacagagggc

SEQ ID NO: 196 - DNA, 20 bp, Artificial sequence
  Forward primer
cctacattttcttcgctccc

SEQ ID NO: 197 - DNA, 20 bp, Artificial sequence
  Reverse primer
aaaaggagggaagtagcacc

SEQ ID NO: 198 - DNA, 20 bp, Artificial sequence
  Forward primer
tgtaaaacgggaagtgcagg

SEQ ID NO: 199 - DNA, 20 bp, Artificial sequence
  Reverse primer
actatggctgtgaggtcatg

SEQ ID NO: 200 - DNA, 20 bp, Artificial sequence
  Forward primer
tgtaaaacgggaagtgcagg

SEQ ID NO: 201 - DNA, 20 bp, Artificial sequence
  Reverse primer
actatggctgtgaggtcatg

SEQ ID NO: 202 - DNA, 20 bp, Artificial sequence
  Forward primer
tgactagcctgcagatcttg

SEQ ID NO: 203 - DNA, 20 bp, Artificial sequence
  Reverse primer
tgaacagtcagaggtcacac

SEQ ID NO: 204 - DNA, 20 bp, Artificial sequence
  Forward primer
tgactagcctgcagatcttg

SEQ ID NO: 205 - DNA, 20 bp, Artificial sequence
  Reverse primer
tgaacagtcagaggtcacac

SEQ ID NO: 206 - DNA, 20 bp, Artificial sequence
  Forward primer
ataccgctctgggtctcacc

SEQ ID NO: 207 - DNA, 20 bp, Artificial sequence
  Reverse primer
gaaccaaagcccaaaagccc

SEQ ID NO: 208 - DNA, 20 bp, Artificial sequence
  Forward primer
ataccgctctgggtctcacc

SEQ ID NO: 209 - DNA, 20 bp, Artificial sequence
  Reverse primer
gaaccaaagcccaaaagccc

SEQ ID NO: 210 - DNA, 20 bp, Artificial sequence
  Forward primer
acagatatcagtgtctcccc

SEQ ID NO: 211 - DNA, 20 bp, Artificial sequence
  Reverse primer
gaaagctacaggcatggaag

SEQ ID NO: 212 - DNA, 20 bp, Artificial sequence
  Forward primer
ttccactgagaaggtaggag

SEQ ID NO: 213 - DNA, 20 bp, Artificial sequence
  Reverse primer
ggcaacaaaagggacttctc

SEQ ID NO: 214 - DNA, 20 bp, Artificial sequence
  Forward primer
aatcacagcgccatctatcc

SEQ ID NO: 215 - DNA, 20 bp, Artificial sequence
  Reverse primer
tctgttcctctcttcctctg

SEQ ID NO: 216 - DNA, 20 bp, Artificial sequence
  Forward primer
acagagactatcaaactagg

SEQ ID NO: 217 - DNA, 20 bp, Artificial sequence
  Reverse primer
ttcttttattcctgcccctg

SEQ ID NO: 218 - DNA, 20 bp, Artificial sequence
  Forward primer
gctaactcctctgaaaagcc

SEQ ID NO: 219 - DNA, 20 bp, Artificial sequence
  Reverse primer
aaaaagggtctgaggctgag

SEQ ID NO: 220 - DNA, 21 bp, Artificial sequence
  Forward primer
ctgtagaaagagagaaatcag

SEQ ID NO: 221 - DNA, 20 bp, Artificial sequence
  Reverse primer
taggacacatagggtttgag

SEQ ID NO: 222 - DNA, 20 bp, Artificial sequence
  Forward primer
agtagaactggcttcaggtg

SEQ ID NO: 223 - DNA, 20 bp, Artificial sequence
  Reverse primer
catctgccaagctattgtcc

SEQ ID NO: 224 - DNA, 20 bp, Artificial sequence
  Forward primer
ccctcagcctctgtttattc

SEQ ID NO: 225 - DNA, 20 bp, Artificial sequence
  Reverse primer
aggccctgatctaagtcttg

SEQ ID NO: 226 - DNA, 20 bp, Artificial sequence
  Forward primer
tccatagcagaaagggacag

SEQ ID NO: 227 - DNA, 20 bp, Artificial sequence
  Reverse primer
ctcttcactttccacacacc

SEQ ID NO: 228 - DNA, 20 bp, Artificial sequence
  Forward primer
ccaccaacaattcagttacc

SEQ ID NO: 229 - DNA, 20 bp, Artificial sequence
  Reverse primer
tactactgcattcacagttc

SEQ ID NO: 230 - DNA, 20 bp, Artificial sequence
  Forward primer
tcaagggttccttctcagag

SEQ ID NO: 231 - DNA, 20 bp, Artificial sequence
  Reverse primer
tctacagacggatactccac

SEQ ID NO: 232 - DNA, 20 bp, Artificial sequence
  Forward primer
tttgtacctgaggaggagag

SEQ ID NO: 233 - DNA, 20 bp, Artificial sequence
  Reverse primer
gttggtctcaatctccgttc

SEQ ID NO: 234 - DNA, 20 bp, Artificial sequence
  Forward primer
acagaggcagtcaattctgc

SEQ ID NO: 235 - DNA, 20 bp, Artificial sequence
  Reverse primer
tgtgtgcactcctacacatc

SEQ ID NO: 236 - DNA, 20 bp, Artificial sequence
  Forward primer
ttaatggtcctggaggacac

SEQ ID NO: 237 - DNA, 20 bp, Artificial sequence
  Reverse primer
caggacacgtactagattag

SEQ ID NO: 238 - DNA, 20 bp, Artificial sequence
  Forward primer
atgtccgtcagaacccatgc

SEQ ID NO: 239 - DNA, 20 bp, Artificial sequence
  Reverse primer
tggatgcagcccgccattag

SEQ ID NO: 240 - DNA, 20 bp, Artificial sequence
  Forward primer
tactgcccacttctcaacag

SEQ ID NO: 241 - DNA, 20 bp, Artificial sequence
  Reverse primer
ggacagatgactgggttttg

SEQ ID NO: 242 - DNA, 20 bp, Artificial sequence
  Forward primer
tcttgaagaccagctgaccc

SEQ ID NO: 243 - DNA, 20 bp, Artificial sequence
  Reverse primer
actggctgcagatcttggac

SEQ ID NO: 244 - DNA, 20 bp, Artificial sequence
  Forward primer
cagacatgccaaacgaattg

SEQ ID NO: 245 - DNA, 20 bp, Artificial sequence
  Reverse primer
agatcaggcatgacctgaac

SEQ ID NO: 246 - DNA, 20 bp, Artificial sequence
  Forward primer
tagagctttgtgccactgag

SEQ ID NO: 247 - DNA, 20 bp, Artificial sequence
  Reverse primer
cccatttcacatcttcctcc

SEQ ID NO: 248 - DNA, 20 bp, Artificial sequence
  Forward primer
gctgctgtttgatctacctg

SEQ ID NO: 249 - DNA, 20 bp, Artificial sequence
  Reverse primer
gggaaacaaccaatgttggg

SEQ ID NO: 250 - DNA, 20 bp, Artificial sequence
  Forward primer
agcccttccaggcaactttg

SEQ ID NO: 251 - DNA, 20 bp, Artificial sequence
  Reverse primer
cttcccttaaaattctatcc

SEQ ID NO: 252 - DNA, 20 bp, Artificial sequence
  Forward primer
agtgaaggacagctgatgtc

SEQ ID NO: 253 - DNA, 20 bp, Artificial sequence
  Reverse primer
gcttcagagctagaaagagc

SEQ ID NO: 254 - DNA, 20 bp, Artificial sequence
  Forward primer
attgcccttattgggccttg

SEQ ID NO: 255 - DNA, 20 bp, Artificial sequence
  Reverse primer
tgggagcaatgtcatcaacc

SEQ ID NO: 256 - DNA, 20 bp, Artificial sequence
  Forward primer
gctcttcactacaaaagtgc

SEQ ID NO: 257 - DNA, 20 bp, Artificial sequence
  Reverse primer
cttctgcagtttcttctgag

SEQ ID NO: 258 - DNA, 20 bp, Artificial sequence
  Forward primer
actctgacccatctacactg

SEQ ID NO: 259 - DNA, 20 bp, Artificial sequence
  Reverse primer
ataacacacagaaccctggg

SEQ ID NO: 260 - DNA, 21 bp, Artificial sequence
  Forward primer
caacagtcttgagaatagatg

SEQ ID NO: 261 - DNA, 21 bp, Artificial sequence
  Reverse primer
gtttatttctagatggcaagg

SEQ ID NO: 262 - DNA, 20 bp, Artificial sequence
  Forward primer
aaggtggggtctcgagagag

SEQ ID NO: 263 - DNA, 19 bp, Artificial sequence
  Reverse primer
catgaatgacccacgagtc

SEQ ID NO: 264 - DNA, 20 bp, Artificial sequence
  Forward primer
ttctccttcacaaaggctgc

SEQ ID NO: 265 - DNA, 20 bp, Artificial sequence
  Reverse primer
ggtatagatcctgaatcctg

SEQ ID NO: 266 - DNA, 20 bp, Artificial sequence
  Forward primer
ttcactcccttgtgctgatg

SEQ ID NO: 267 - DNA, 20 bp, Artificial sequence
  Reverse primer
gggaactatccagatggttg

SEQ ID NO: 268 - DNA, 20 bp, Artificial sequence
  Forward primer
caaccagctgggtattttgc

SEQ ID NO: 269 - DNA, 20 bp, Artificial sequence
  Reverse primer
taagccttgttccaggttgg

SEQ ID NO: 270 - DNA, 20 bp, Artificial sequence
  Forward primer
tagagcccaggtactagttc

SEQ ID NO: 271 - DNA, 20 bp, Artificial sequence
  Reverse primer
tcttgaagtggagactgagg

SEQ ID NO: 272 - DNA, 20 bp, Artificial sequence
  Forward primer
aagggctgagtttggtagtg

SEQ ID NO: 273 - DNA, 20 bp, Artificial sequence
  Reverse primer
agctgtagggactcatcttg

SEQ ID NO: 274 - DNA, 20 bp, Artificial sequence
  Forward primer
catcatttgtgcttgcggtg

SEQ ID NO: 275 - DNA, 20 bp, Artificial sequence
  Reverse primer
aaggcctcaattcacggtag

SEQ ID NO: 276 - DNA, 20 bp, Artificial sequence
  Forward primer
catcatttgtgcttgcggtg

SEQ ID NO: 277 - DNA, 20 bp, Artificial sequence
  Reverse primer
aaggcctcaattcacggtag

SEQ ID NO: 278 - DNA, 20 bp, Artificial sequence
  Forward primer
tgccaccttctttttctccc

SEQ ID NO: 279 - DNA, 20 bp, Artificial sequence
  Reverse primer
agtctgccagcttcaaatcc

SEQ ID NO: 280 - DNA, 19 bp, Artificial sequence
  Forward primer
acaggctgccatagctggg

SEQ ID NO: 281 - DNA, 20 bp, Artificial sequence
  Reverse primer
cacactctctcgagcctacc

SEQ ID NO: 282 - DNA, 20 bp, Artificial sequence
  Forward primer
aaagtgctgggattacaggc

SEQ ID NO: 283 - DNA, 20 bp, Artificial sequence
  Reverse primer
tgttcagctagtagcacctc

SEQ ID NO: 284 - DNA, 20 bp, Artificial sequence
  Forward primer
atggcgaggccagcaggtga

SEQ ID NO: 285 - DNA, 19 bp, Artificial sequence
  Reverse primer
gaggacggccccatggcac

SEQ ID NO: 286 - DNA, 20 bp, Artificial sequence
  Forward primer
ctattgatttcttccacagg

SEQ ID NO: 287 - DNA, 20 bp, Artificial sequence
  Reverse primer
taaagatggcaaggtaaagc

SEQ ID NO: 288 - DNA, 20 bp, Artificial sequence
  Forward primer
gacagagctagactcattcc

SEQ ID NO: 289 - DNA, 20 bp, Artificial sequence
  Reverse primer
tccctgatctttggcagcag

SEQ ID NO: 290 - DNA, 20 bp, Artificial sequence
  Forward primer
ataagagaaggggtctgcgg

SEQ ID NO: 291 - DNA, 21 bp, Artificial sequence
  Reverse primer
ttccgcctcctttcgggcttt

SEQ ID NO: 292 - DNA, 19 bp, Artificial sequence
  Forward primer
caggaggtgccagtgagac

SEQ ID NO: 293 - DNA, 20 bp, Artificial sequence
  Reverse primer
aggacaccatgatgtcccag

SEQ ID NO: 294 - DNA, 20 bp, Artificial sequence
  Forward primer
cctatctgggcatatcccag

SEQ ID NO: 295 - DNA, 20 bp, Artificial sequence
  Reverse primer
aggcatcaaagcccttacag

SEQ ID NO: 296 - DNA, 20 bp, Artificial sequence
  Forward primer
acttctgggcactgagcacc

SEQ ID NO: 297 - DNA, 19 bp, Artificial sequence
  Reverse primer
tgtacgccacctgcccctg

SEQ ID NO: 298 - DNA, 20 bp, Artificial sequence
  Forward primer
aaaatatctccctggacggg

SEQ ID NO: 299 - DNA, 20 bp, Artificial sequence
  Reverse primer
gacacagaagcaatgatggg

SEQ ID NO: 300 - DNA, 20 bp, Artificial sequence
  Forward primer
acagaggcaggcatgtgcag

SEQ ID NO: 301 - DNA, 20 bp, Artificial sequence
  Reverse primer
caggtctcaggaagcatgtg

SEQ ID NO: 302 - DNA, 20 bp, Artificial sequence
  Forward primer
aaggggccaggtgggcagga

SEQ ID NO: 303 - DNA, 20 bp, Artificial sequence
  Reverse primer
atgtgtctccccgtgagtcg

SEQ ID NO: 304 - DNA, 20 bp, Artificial sequence
  Forward primer
aaggggccaggtgggcagga

SEQ ID NO: 305 - DNA, 20 bp, Artificial sequence
  Reverse primer
atgtgtctccccgtgagtcg

SEQ ID NO: 306 - DNA, 20 bp, Artificial sequence
  Forward primer
ttatgcacggcaaagtccag

SEQ ID NO: 307 - DNA, 20 bp, Artificial sequence
  Reverse primer
aaacaagagatgctctgccg

SEQ ID NO: 308 - DNA, 20 bp, Artificial sequence
  Forward primer
gcaatagtgaaggctagtgg

SEQ ID NO: 309 - DNA, 20 bp, Artificial sequence
  Reverse primer
gcaccagacactccctaaaa

SEQ ID NO: 310 - DNA, 20 bp, Artificial sequence
  Forward primer
gcaatagtgaaggctagtgg

SEQ ID NO: 311 - DNA, 20 bp, Artificial sequence
  Reverse primer
gcaccagacactccctaaaa

SEQ ID NO: 312 - DNA, 20 bp, Artificial sequence
  Forward primer
gaggcatccatgttaccatg

SEQ ID NO: 313 - DNA, 20 bp, Artificial sequence
  Reverse primer
gccattggttctgcagtaag

SEQ ID NO: 314 - DNA, 20 bp, Artificial sequence
  Forward primer
tttgtacttgtttgccggcc

SEQ ID NO: 315 - DNA, 20 bp, Artificial sequence
  Reverse primer
gaaatcctgctctgcctttc

SEQ ID NO: 316 - DNA, 20 bp, Artificial sequence
  Forward primer
cctcttgattctctgtctgc

SEQ ID NO: 317 - DNA, 20 bp, Artificial sequence
  Reverse primer
aggcagtaactaagtgtgag

SEQ ID NO: 318 - DNA, 20 bp, Artificial sequence
  Forward primer
atgttagactgtcctcccag

SEQ ID NO: 319 - DNA, 20 bp, Artificial sequence
  Reverse primer
gattgaattctgcgaaaggc

SEQ ID NO: 320 - DNA, 20 bp, Artificial sequence
  Forward primer
agtggatagaggctgatgcc

SEQ ID NO: 321 - DNA, 20 bp, Artificial sequence
  Reverse primer
aaggacctcccgcttcgcac

SEQ ID NO: 322 - DNA, 20 bp, Artificial sequence
  Forward primer
tagcaacctctgtagcacac

SEQ ID NO: 323 - DNA, 20 bp, Artificial sequence
  Reverse primer
tggtcttcgtgaccttcttg

SEQ ID NO: 324 - DNA, 20 bp, Artificial sequence
  Forward primer
tgcagagcccactctggatg

SEQ ID NO: 325 - DNA, 20 bp, Artificial sequence
  Reverse primer
ctcaagctggagatgagcac

SEQ ID NO: 326 - DNA, 20 bp, Artificial sequence
  Forward primer
acatccacgctcagcgtgaa

SEQ ID NO: 327 - DNA, 20 bp, Artificial sequence
  Reverse primer
cctacaggtgaagctagatg

SEQ ID NO: 328 - DNA, 20 bp, Artificial sequence
  Forward primer
atgagagaagtgacagtggc

SEQ ID NO: 329 - DNA, 20 bp, Artificial sequence
  Reverse primer
cagactcaaaaccctcactc

SEQ ID NO: 330 - DNA, 20 bp, Artificial sequence
  Forward primer
atgagagaagtgacagtggc

SEQ ID NO: 331 - DNA, 20 bp, Artificial sequence
  Reverse primer
cagactcaaaaccctcactc

SEQ ID NO: 332 - DNA, 20 bp, Artificial sequence
  Forward primer
tcatgttgttaccctcggac

SEQ ID NO: 333 - DNA, 20 bp, Artificial sequence
  Reverse primer
tcaaaggtccttctgtccag

SEQ ID NO: 334 - DNA, 20 bp, Artificial sequence
  Forward primer
acagtgccatggcgggacaa

SEQ ID NO: 335 - DNA, 19 bp, Artificial sequence
  Reverse primer
aaaccaggccgcccttccc

SEQ ID NO: 336 - DNA, 20 bp, Artificial sequence
  Forward primer
aggaactgatgaggtaaggc

SEQ ID NO: 337 - DNA, 20 bp, Artificial sequence
  Reverse primer
caaatccattgagggctcac

SEQ ID NO: 338 - DNA, 20 bp, Artificial sequence
  Forward primer
ggtagggagaaatcgaaggg

SEQ ID NO: 339 - DNA, 19 bp, Artificial sequence
  Reverse primer
gggttgtccctctcgcagc

SEQ ID NO: 340 - DNA, 20 bp, Artificial sequence
  Forward primer
tttctcctccttccttgggc

SEQ ID NO: 341 - DNA, 20 bp, Artificial sequence
  Reverse primer
aaagagaggaacccccactg

SEQ ID NO: 342 - DNA, 20 bp, Artificial sequence
  Forward primer
atcggagagatggtggagag

SEQ ID NO: 343 - DNA, 20 bp, Artificial sequence
  Reverse primer
tgcaaatctgtccccatcag

SEQ ID NO: 344 - DNA, 20 bp, Artificial sequence
  Forward primer
gcctttccacaggattattg

SEQ ID NO: 345 - DNA, 21 bp, Artificial sequence
  Reverse primer
ctagtggaaggtattacaacc

SEQ ID NO: 346 - DNA, 20 bp, Artificial sequence
  Forward primer
ttctagtccccaaccatgtg

SEQ ID NO: 347 - DNA, 20 bp, Artificial sequence
  Reverse primer
accagaatcacttgcacagc

SEQ ID NO: 348 - DNA, 20 bp, Artificial sequence
  Forward primer
tgtctcccacatggacgatg

SEQ ID NO: 349 - DNA, 21 bp, Artificial sequence
  Reverse primer
agtaggtacgcacctgacctc

SEQ ID NO: 350 - DNA, 20 bp, Artificial sequence
  Forward primer
tctctgctttctcccttggc

SEQ ID NO: 351 - DNA, 20 bp, Artificial sequence
  Reverse primer
accaacagacaagtgtggtc

SEQ ID NO: 352 - DNA, 20 bp, Artificial sequence
  Forward primer
atgagcttgacacctccttc

SEQ ID NO: 353 - DNA, 20 bp, Artificial sequence
  Reverse primer
cccattgacatatccgcgag

SEQ ID NO: 354 - DNA, 19 bp, Artificial sequence
  Forward primer
tagtgccaggtgcagccac

SEQ ID NO: 355 - DNA, 20 bp, Artificial sequence
  Reverse primer
acagacacacgtggcatctc

SEQ ID NO: 356 - DNA, 19 bp, Artificial sequence
  Forward primer
tagtgccaggtgcagccac

SEQ ID NO: 357 - DNA, 20 bp, Artificial sequence
  Reverse primer
acagacacacgtggcatctc

SEQ ID NO: 358 - DNA, 20 bp, Artificial sequence
  Forward primer
accttgcaagcatcccatgg

SEQ ID NO: 359 - DNA, 20 bp, Artificial sequence
  Reverse primer
atgaaaccagacagtgtccc

SEQ ID NO: 360 - DNA, 20 bp, Artificial sequence
  Forward primer
ggtatttggcctcaacaagc

SEQ ID NO: 361 - DNA, 20 bp, Artificial sequence
  Reverse primer
caagatgagatttgggtggg

SEQ ID NO: 362 - DNA, 20 bp, Artificial sequence
  Forward primer
ggtatttggcctcaacaagc

SEQ ID NO: 363 - DNA, 20 bp, Artificial sequence
  Reverse primer
caagatgagatttgggtggg

SEQ ID NO: 364 - DNA, 20 bp, Artificial sequence
  Forward primer
tacactgctcttcccaagtc

SEQ ID NO: 365 - DNA, 20 bp, Artificial sequence
  Reverse primer
gcatccacaactagaaccac

SEQ ID NO: 366 - DNA, 19 bp, Artificial sequence
  Forward primer
gctggggcgcagggctaga

SEQ ID NO: 367 - DNA, 20 bp, Artificial sequence
  Reverse primer
ttcacggaaacgacagctgc

SEQ ID NO: 368 - DNA, 20 bp, Artificial sequence
  Forward primer
actaactgcaggtctatgtg

SEQ ID NO: 369 - DNA, 20 bp, Artificial sequence
  Reverse primer
tgtggatgtgggtgacaacg

SEQ ID NO: 370 - DNA, 20 bp, Artificial sequence
  Forward primer
actaactgcaggtctatgtg

SEQ ID NO: 371 - DNA, 20 bp, Artificial sequence
  Reverse primer
tgtggatgtgggtgacaacg

SEQ ID NO: 372 - DNA, 20 bp, Artificial sequence
  Forward primer
atttaagcgggctgtgaacg

SEQ ID NO: 373 - DNA, 20 bp, Artificial sequence
  Reverse primer
tcacttatagagcctgcctg

SEQ ID NO: 374 - DNA, 20 bp, Artificial sequence
  Forward primer
agagccaggttttgtacacg

SEQ ID NO: 375 - DNA, 20 bp, Artificial sequence
  Reverse primer
tttatcctgcggcttctctg

SEQ ID NO: 376 - DNA, 20 bp, Artificial sequence
  Forward primer
agaccaaatgtcatctcccc

SEQ ID NO: 377 - DNA, 20 bp, Artificial sequence
  Reverse primer
ttttctccacaaaccagacc

SEQ ID NO: 378 - DNA, 20 bp, Artificial sequence
  Forward primer
cctgctttgctgttcctaag

SEQ ID NO: 379 - DNA, 20 bp, Artificial sequence
  Reverse primer
acttagattgagggctcctg

SEQ ID NO: 380 - DNA, 20 bp, Artificial sequence
  Forward primer
ttgggtttccaagacagagc

SEQ ID NO: 381 - DNA, 20 bp, Artificial sequence
  Reverse primer
gaggagaaagcatcgagttc

SEQ ID NO: 382 - DNA, 20 bp, Artificial sequence
  Forward primer
agcctcgagcctttgctttc

SEQ ID NO: 383 - DNA, 20 bp, Artificial sequence
  Reverse primer
atctagagagtgccaggcac

SEQ ID NO: 384 - DNA, 21 bp, Artificial sequence
  Forward primer
ggctctctattatatgctctc

SEQ ID NO: 385 - DNA, 20 bp, Artificial sequence
  Reverse primer
tgaccttagaaccgtatctg

SEQ ID NO: 386 - DNA, 20 bp, Artificial sequence
  Forward primer
gagtctatgaatcactcagg

SEQ ID NO: 387 - DNA, 20 bp, Artificial sequence
  Reverse primer
ggtggttttggctaattagg

SEQ ID NO: 388 - DNA, 20 bp, Artificial sequence
  Forward primer
atggtcaacagtgttgccag

SEQ ID NO: 389 - DNA, 20 bp, Artificial sequence
  Reverse primer
atcagtgctgctcttctcag

SEQ ID NO: 390 - DNA, 20 bp, Artificial sequence
  Forward primer
actgggcaggaagtctcacc

SEQ ID NO: 391 - DNA, 19 bp, Artificial sequence
  Reverse primer
gggctctttcccaagtgcc

SEQ ID NO: 392 - DNA, 20 bp, Artificial sequence
  Forward primer
cacctcttgattgctttccc

SEQ ID NO: 393 - DNA, 20 bp, Artificial sequence
  Reverse primer
aattcagccacaatcacccg

SEQ ID NO: 394 - DNA, 20 bp, Artificial sequence
  Forward primer
agtggtactgctgggaaatg

SEQ ID NO: 395 - DNA, 20 bp, Artificial sequence
  Reverse primer
gcgaggtttttaggatgacc

SEQ ID NO: 396 - DNA, 20 bp, Artificial sequence
  Forward primer
ccaacctacaacctgtgtac

SEQ ID NO: 397 - DNA, 20 bp, Artificial sequence
  Reverse primer
tagggctaggaatagaagtc

SEQ ID NO: 398 - DNA, 20 bp, Artificial sequence
  Forward primer
cttcctctccagcttaagac

SEQ ID NO: 399 - DNA, 20 bp, Artificial sequence
  Reverse primer
ggctttagattggaggcatg

SEQ ID NO: 400 - DNA, 20 bp, Artificial sequence
  Forward primer
ttagccgtgtacttttggag

SEQ ID NO: 401 - DNA, 20 bp, Artificial sequence
  Reverse primer
ctaagcgtcagaaattaccc

SEQ ID NO: 402 - DNA, 20 bp, Artificial sequence
  Forward primer
tctctcagttgtatatcatg

SEQ ID NO: 403 - DNA, 20 bp, Artificial sequence
  Reverse primer
tgctagataaggtatccatg

SEQ ID NO: 404 - DNA, 20 bp, Artificial sequence
  Forward primer
tttggcaggatcctgaggag

SEQ ID NO: 405 - DNA, 20 bp, Artificial sequence
  Reverse primer
tttctctcttcagcctcagg

SEQ ID NO: 406 - DNA, 20 bp, Artificial sequence
  Forward primer
tttctgctataccagggcac

SEQ ID NO: 407 - DNA, 20 bp, Artificial sequence
  Reverse primer
gatgtgagcgagttctgtag

SEQ ID NO: 408 - DNA, 21 bp, Artificial sequence
  Forward primer
gtcttcaactctctaattctc

SEQ ID NO: 409 - DNA, 20 bp, Artificial sequence
  Reverse primer
caacaaaggcaaggaagtag

SEQ ID NO: 410 - DNA, 20 bp, Artificial sequence
  Forward primer
ttccaggcaacaacagctag

SEQ ID NO: 411 - DNA, 20 bp, Artificial sequence
  Reverse primer
tcaaaacagaccactgggag

SEQ ID NO: 412 - DNA, 20 bp, Artificial sequence
  Forward primer
ttgattccaagtacagcagc

SEQ ID NO: 413 - DNA, 20 bp, Artificial sequence
  Reverse primer
gtcccaataacaccctagac

SEQ ID NO: 414 - DNA, 20 bp, Artificial sequence
  Forward primer
acagaccagacacctggcag

SEQ ID NO: 415 - DNA, 20 bp, Artificial sequence
  Reverse primer
ctgcctggaggaattctcag

SEQ ID NO: 416 - DNA, 20 bp, Artificial sequence
  Forward primer
attctctgagggcgggaatc

SEQ ID NO: 417 - DNA, 20 bp, Artificial sequence
  Reverse primer
aacggggaaaccgaggtatg

SEQ ID NO: 418 - DNA, 20 bp, Artificial sequence
  Forward primer
gtcctcccacaacaatgtcc

SEQ ID NO: 419 - DNA, 20 bp, Artificial sequence
  Reverse primer
ctgtctctgggcgtgcatac

SEQ ID NO: 420 - DNA, 20 bp, Artificial sequence
  Forward primer
tggtccccgagaataagatg

SEQ ID NO: 421 - DNA, 20 bp, Artificial sequence
  Reverse primer
cctcagaagccggatttcag

SEQ ID NO: 422 - DNA, 20 bp, Artificial sequence
  Forward primer
aggaagcaggaaggagaatg

SEQ ID NO: 423 - DNA, 20 bp, Artificial sequence
  Reverse primer
ctgcttccgtttctcattac

SEQ ID NO: 424 - DNA, 20 bp, Artificial sequence
  Forward primer
cacgccgatcctcaaacgag

SEQ ID NO: 425 - DNA, 19 bp, Artificial sequence
  Reverse primer
agggtcaggacggtgcaac

SEQ ID NO: 426 - DNA, 21 bp, Artificial sequence
  Forward primer
ggataccttatctagcattgc

SEQ ID NO: 427 - DNA, 20 bp, Artificial sequence
  Reverse primer
attggtttgagcaaaggagc

SEQ ID NO: 428 - DNA, 20 bp, Artificial sequence
  Forward primer
gatgactaataagggccgag

SEQ ID NO: 429 - DNA, 20 bp, Artificial sequence
  Reverse primer
catggcagaagagatggaac

SEQ ID NO: 430 - DNA, 20 bp, Artificial sequence
  Forward primer
ccaaaggactgctcagtttc

SEQ ID NO: 431 - DNA, 20 bp, Artificial sequence
  Reverse primer
aaccactcgtggagcttttg

SEQ ID NO: 432 - DNA, 21 bp, Artificial sequence
  Forward primer
cttagattgaaagtgtacccc

SEQ ID NO: 433 - DNA, 20 bp, Artificial sequence
  Reverse primer
gtgactaggtatggagttac

SEQ ID NO: 434 - DNA, 20 bp, Artificial sequence
  Forward primer
tttccgtacatgtcctggtg

SEQ ID NO: 435 - DNA, 20 bp, Artificial sequence
  Reverse primer
ttgaagtggtcaggcaatgg

SEQ ID NO: 436 - DNA, 20 bp, Artificial sequence
  Forward primer
actgtggctactcagctgtg

SEQ ID NO: 437 - DNA, 20 bp, Artificial sequence
  Reverse primer
cagtcggggttgatctcgat

SEQ ID NO: 438 - DNA, 20 bp, Artificial sequence
  Forward primer
actgtggctactcagctgtg

SEQ ID NO: 439 - DNA, 20 bp, Artificial sequence
  Reverse primer
cagtcggggttgatctcgat

SEQ ID NO: 440 - DNA, 20 bp, Artificial sequence
  Forward primer
tcctgctcgttttgtctatc

SEQ ID NO: 441 - DNA, 20 bp, Artificial sequence
  Reverse primer
tggataagggcagagacatg

SEQ ID NO: 442 - DNA, 20 bp, Artificial sequence
  Forward primer
attcacagcaagttgaactc

SEQ ID NO: 443 - DNA, 20 bp, Artificial sequence
  Reverse primer
atcgttttggatgagtcatg

SEQ ID NO: 444 - DNA, 20 bp, Artificial sequence
  Forward primer
ctagtgctgctgagggactc

SEQ ID NO: 445 - DNA, 20 bp, Artificial sequence
  Reverse primer
tgacttgagcggcaggaaag

SEQ ID NO: 446 - DNA, 20 bp, Artificial sequence
  Forward primer
tgaagttctcagagaggagg

SEQ ID NO: 447 - DNA, 20 bp, Artificial sequence
  Reverse primer
ttgcaagtctgaacacaggg

SEQ ID NO: 448 - DNA, 20 bp, Artificial sequence
  Forward primer
aaggagggtggaaagaatgg

SEQ ID NO: 449 - DNA, 20 bp, Artificial sequence
  Reverse primer
ataatggtgagagcaccagc

SEQ ID NO: 450 - DNA, 20 bp, Artificial sequence
  Forward primer
aaggagggtggaaagaatgg

SEQ ID NO: 451 - DNA, 20 bp, Artificial sequence
  Reverse primer
ataatggtgagagcaccagc

SEQ ID NO: 452 - DNA, 20 bp, Artificial sequence
  Forward primer
aggagtccatagaaacggac

SEQ ID NO: 453 - DNA, 20 bp, Artificial sequence
  Reverse primer
ttgcttcctctgggaacttg

SEQ ID NO: 454 - DNA, 20 bp, Artificial sequence
  Forward primer
tcattcagcagtggaagcag

SEQ ID NO: 455 - DNA, 20 bp, Artificial sequence
  Reverse primer
agtcatgatggcatggatgg

SEQ ID NO: 456 - DNA, 20 bp, Artificial sequence
  Forward primer
aaggccacctagcaggacca

SEQ ID NO: 457 - DNA, 20 bp, Artificial sequence
  Reverse primer
cagctagcctcacagtgaac

SEQ ID NO: 458 - DNA, 20 bp, Artificial sequence
  Forward primer
tccgtaccttgctcatgcag

SEQ ID NO: 459 - DNA, 20 bp, Artificial sequence
  Reverse primer
atgatgaacaccgccaagac

SEQ ID NO: 460 - DNA, 20 bp, Artificial sequence
  Forward primer
aagctacagtaatccagacg

SEQ ID NO: 461 - DNA, 20 bp, Artificial sequence
  Reverse primer
catgcttcagagttgactag

SEQ ID NO: 462 - DNA, 20 bp, Artificial sequence
  Forward primer
tcaagaacctcaaacgtggc

SEQ ID NO: 463 - DNA, 20 bp, Artificial sequence
  Reverse primer
actctatgttcacctgcagc

SEQ ID NO: 464 - DNA, 20 bp, Artificial sequence
  Forward primer
gaaccttgagtggaacctag

SEQ ID NO: 465 - DNA, 20 bp, Artificial sequence
  Reverse primer
actagagtatgagtgtcacc

SEQ ID NO: 466 - DNA, 20 bp, Artificial sequence
  Forward primer
taggtgtcaatggcctccag

SEQ ID NO: 467 - DNA, 20 bp, Artificial sequence
  Reverse primer
tcatgggtgacaccaaggag

SEQ ID NO: 468 - DNA, 20 bp, Artificial sequence
  Forward primer
aatcaccacaccaaggccga

SEQ ID NO: 469 - DNA, 20 bp, Artificial sequence
  Reverse primer
tatggctagggaaagcactg

SEQ ID NO: 470 - DNA, 20 bp, Artificial sequence
  Forward primer
tgaggaaaaaaacaaccccc

SEQ ID NO: 471 - DNA, 20 bp, Artificial sequence
  Reverse primer
aaagatgggaactatttggc

SEQ ID NO: 472 - DNA, 20 bp, Artificial sequence
  Forward primer
ttcctacagccatttgaggg

SEQ ID NO: 473 - DNA, 20 bp, Artificial sequence
  Reverse primer
catcccgtggtcactaaatc

SEQ ID NO: 474 - DNA, 20 bp, Artificial sequence
  Forward primer
attcacctcctgcctcagtc

SEQ ID NO: 475 - DNA, 20 bp, Artificial sequence
  Reverse primer
agcattgaaggaacagggtc

SEQ ID NO: 476 - DNA, 20 bp, Artificial sequence
  Forward primer
aactagtctggccctcattc

SEQ ID NO: 477 - DNA, 20 bp, Artificial sequence
  Reverse primer
ttttccctgctcaggttcag

SEQ ID NO: 478 - DNA, 20 bp, Artificial sequence
  Forward primer
tggcaaggtgagtatgttgg

SEQ ID NO: 479 - DNA, 20 bp, Artificial sequence
  Reverse primer
agcactcagaaaactcactg

SEQ ID NO: 480 - DNA, 20 bp, Artificial sequence
  Forward primer
cagcatctagagtcaaacac

SEQ ID NO: 481 - DNA, 20 bp, Artificial sequence
  Reverse primer
cagcatactacacatgagag

SEQ ID NO: 482 - DNA, 20 bp, Artificial sequence
  Forward primer
tcattcgctattcctcaccc

SEQ ID NO: 483 - DNA, 20 bp, Artificial sequence
  Reverse primer
atgctggctggaaggaaatg

SEQ ID NO: 484 - DNA, 20 bp, Artificial sequence
  Forward primer
tcattcgctattcctcaccc

SEQ ID NO: 485 - DNA, 20 bp, Artificial sequence
  Reverse primer
atgctggctggaaggaaatg

SEQ ID NO: 486 - DNA, 20 bp, Artificial sequence
  Forward primer
acaccaagggaacttctgag

SEQ ID NO: 487 - DNA, 20 bp, Artificial sequence
  Reverse primer
atccgtgcgcagctagaact

SEQ ID NO: 488 - DNA, 20 bp, Artificial sequence
  Forward primer
acaccaagggaacttctgag

SEQ ID NO: 489 - DNA, 20 bp, Artificial sequence
  Reverse primer
atccgtgcgcagctagaact

SEQ ID NO: 490 - DNA, 20 bp, Artificial sequence
  Forward primer
tttcattctccccccacgac

SEQ ID NO: 491 - DNA, 20 bp, Artificial sequence
  Reverse primer
accggaacaaagtaagggcc

SEQ ID NO: 492 - DNA, 20 bp, Artificial sequence
  Forward primer
tgttccatctcctctgcttc

SEQ ID NO: 493 - DNA, 20 bp, Artificial sequence
  Reverse primer
gggtgctgtttgaattggtc

SEQ ID NO: 494 - DNA, 20 bp, Artificial sequence
  Forward primer
atgcaatgcagaatgtggcc

SEQ ID NO: 495 - DNA, 20 bp, Artificial sequence
  Reverse primer
aatgcttttctgctctcccc

SEQ ID NO: 496 - DNA, 20 bp, Artificial sequence
  Forward primer
tcctagagaaacttgaggtg

SEQ ID NO: 497 - DNA, 20 bp, Artificial sequence
  Reverse primer
gttctttgtgcctcttgagg

SEQ ID NO: 498 - DNA, 20 bp, Artificial sequence
  Forward primer
gccactgtaaagctgctttc

SEQ ID NO: 499 - DNA, 20 bp, Artificial sequence
  Reverse primer
gggaaatatgggaggcaaac

SEQ ID NO: 500 - DNA, 20 bp, Artificial sequence
  Forward primer
gccactgtaaagctgctttc

SEQ ID NO: 501 - DNA, 20 bp, Artificial sequence
  Reverse primer
gggaaatatgggaggcaaac

SEQ ID NO: 502 - DNA, 20 bp, Artificial sequence
  Forward primer
tgcacttcccgttttacagg

SEQ ID NO: 503 - DNA, 20 bp, Artificial sequence
  Reverse primer
atctttctccaccagcaagg

SEQ ID NO: 504 - DNA, 20 bp, Artificial sequence
  Forward primer
tgcacttcccgttttacagg

SEQ ID NO: 505 - DNA, 20 bp, Artificial sequence
  Reverse primer
atctttctccaccagcaagg

SEQ ID NO: 506 - DNA, 20 bp, Artificial sequence
  Forward primer
aaagtgtttgtggtggtggc

SEQ ID NO: 507 - DNA, 20 bp, Artificial sequence
  Reverse primer
tactgcctgaggatcaaaac

SEQ ID NO: 508 - DNA, 20 bp, Artificial sequence
  Forward primer
tattcttctccgcttcagcc

SEQ ID NO: 509 - DNA, 20 bp, Artificial sequence
  Reverse primer
tgtgtcaatagggacgtcac

SEQ ID NO: 510 - DNA, 20 bp, Artificial sequence
  Forward primer
gcagagttagaactcacagc

SEQ ID NO: 511 - DNA, 20 bp, Artificial sequence
  Reverse primer
cacagaagtgccacagaaac

SEQ ID NO: 512 - DNA, 20 bp, Artificial sequence
  Forward primer
tcggatggataaggtggaac

SEQ ID NO: 513 - DNA, 20 bp, Artificial sequence
  Reverse primer
agaaagcactagaagccctg

SEQ ID NO: 514 - DNA, 20 bp, Artificial sequence
  Forward primer
agggagtgaagaacaggaag

SEQ ID NO: 515 - DNA, 20 bp, Artificial sequence
  Reverse primer
atagatgagcctcagcttcc

SEQ ID NO: 516 - DNA, 20 bp, Artificial sequence
  Forward primer
gctcttcgatcctgtgattc

SEQ ID NO: 517 - DNA, 20 bp, Artificial sequence
  Reverse primer
attccttctctgggactcag

SEQ ID NO: 518 - DNA, 20 bp, Artificial sequence
  Forward primer
ccagtatgactgaggaactg

SEQ ID NO: 519 - DNA, 20 bp, Artificial sequence
  Reverse primer
tgtatatgtgcagagctgcg

SEQ ID NO: 520 - DNA, 23 bp, Artificial sequence
  Extension primer
atgccctctacaaaatggattta

SEQ ID NO: 521 - DNA, 21 bp, Artificial sequence
  Extension primer
acaaaggattgtttcccagtc

SEQ ID NO: 522 - DNA, 22 bp, Artificial sequence
  Extension primer
gctgtgtgtcttcactgtcatc

SEQ ID NO: 523 - DNA, 21 bp, Artificial sequence
  Extension primer
cctgaacttgggaaataaggc

SEQ ID NO: 524 - DNA, 19 bp, Artificial sequence
  Extension primer
caccttcagctccttggac

SEQ ID NO: 525 - DNA, 21 bp, Artificial sequence
  Extension primer
ggagaagggaattttttggag

SEQ ID NO: 526 - DNA, 21 bp, Artificial sequence
  Extension primer
tctttccattgttggctacgt

SEQ ID NO: 527 - DNA, 18 bp, Artificial sequence
  Extension primer
cctcgcgcttccaggact

SEQ ID NO: 528 - DNA, 19 bp, Artificial sequence
  Extension primer
gagatgggaagcactaggg

SEQ ID NO: 529 - DNA, 24 bp, Artificial sequence
  Extension primer
aaaagggagtctgttccagatcag

SEQ ID NO: 530 - DNA, 17 bp, Artificial sequence
  Extension primer
acagagggcaacgtact

SEQ ID NO: 531 - DNA, 21 bp, Artificial sequence
  Extension primer
gggaagtagcacccaaattaa

SEQ ID NO: 532 - DNA, 24 bp, Artificial sequence
  Extension primer
agctctaggctgacctctgagtac

SEQ ID NO: 533 - DNA, 24 bp, Artificial sequence
  Extension primer
agctctaggctgacctctgagtac

SEQ ID NO: 534 - DNA, 21 bp, Artificial sequence
  Extension primer
ctgagggatctgcttcaaacc

SEQ ID NO: 535 - DNA, 21 bp, Artificial sequence
  Extension primer
ctgagggatctgcttcaaacc

SEQ ID NO: 536 - DNA, 21 bp, Artificial sequence
  Extension primer
aaagcccaaaagccccagatc

SEQ ID NO: 537 - DNA, 21 bp, Artificial sequence
  Extension primer
aaagcccaaaagccccagatc

SEQ ID NO: 538 - DNA, 22 bp, Artificial sequence
  Extension primer
gcccaacctcttctgagacttg

SEQ ID NO: 539 - DNA, 21 bp, Artificial sequence
  Extension primer
ccttcacaactcaagcctaga

SEQ ID NO: 540 - DNA, 17 bp, Artificial sequence
  Extension primer
ctctgctcactgcaggc

SEQ ID NO: 541 - DNA, 22 bp, Artificial sequence
  Extension primer
acagcataaaattggatctcac

SEQ ID NO: 542 - DNA, 24 bp, Artificial sequence
  Extension primer
ggctgagaaaattgaggatcaggc

SEQ ID NO: 543 - DNA, 23 bp, Artificial sequence
  Extension primer
aaactcagggaaatttagcaagg

SEQ ID NO: 544 - DNA, 21 bp, Artificial sequence
  Extension primer
caagctattgtccaagcgtga

SEQ ID NO: 545 - DNA, 24 bp, Artificial sequence
  Extension primer
agaaaacgtactttttgaggaaag

SEQ ID NO: 546 - DNA, 19 bp, Artificial sequence
  Extension primer
acagtggtaatttcattgc

SEQ ID NO: 547 - DNA, 21 bp, Artificial sequence
  Extension primer
cagttttcttaagcatgacct

SEQ ID NO: 548 - DNA, 24 bp, Artificial sequence
  Extension primer
cagacggatactccacggtggacc

SEQ ID NO: 549 - DNA, 20 bp, Artificial sequence
  Extension primer
caatctccgttcttcaggct

SEQ ID NO: 550 - DNA, 24 bp, Artificial sequence
  Extension primer
ccccagctcaggacactccactga

SEQ ID NO: 551 - DNA, 23 bp, Artificial sequence
  Extension primer
actagattagcagaaccaaaatc

SEQ ID NO: 552 - DNA, 19 bp, Artificial sequence
  Extension primer
ttagcgcatcacagtcgcg

SEQ ID NO: 553 - DNA, 20 bp, Artificial sequence
  Extension primer
gactgggttttggggagcac

SEQ ID NO: 554 - DNA, 22 bp, Artificial sequence
  Extension primer
tgcagatcttggaccgagtagc

SEQ ID NO: 555 - DNA, 24 bp, Artificial sequence
  Extension primer
cttgaatagaagtgtactaggaag

SEQ ID NO: 556 - DNA, 18 bp, Artificial sequence
  Extension primer
tccgagttccccagcacc

SEQ ID NO: 557 - DNA, 21 bp, Artificial sequence
  Extension primer
aatgttgggtctaaagaatac

SEQ ID NO: 558 - DNA, 25 bp, Artificial sequence
  Extension primer
tttattttaaacctctaacatctca

SEQ ID NO: 559 - DNA, 24 bp, Artificial sequence
  Extension primer
cagagctagaaagagcattaggtg

SEQ ID NO: 560 - DNA, 20 bp, Artificial sequence
  Extension primer
gtcatcaacccaaagggaac

SEQ ID NO: 561 - DNA, 24 bp, Artificial sequence
  Extension primer
tcgatgtgacatattagtctatga

SEQ ID NO: 562 - DNA, 19 bp, Artificial sequence
  Extension primer
gtggaaaagagctcagccc

SEQ ID NO: 563 - DNA, 22 bp, Artificial sequence
  Extension primer
tctagatggcaaggaaaatcac

SEQ ID NO: 564 - DNA, 19 bp, Artificial sequence
  Extension primer
gacccacgagtcttcagcg

SEQ ID NO: 565 - DNA, 21 bp, Artificial sequence
  Extension primer
gatcctgaatcctgtaagtga

SEQ ID NO: 566 - DNA, 24 bp, Artificial sequence
  Extension primer
tatccagatggttgtctaataggt

SEQ ID NO: 567 - DNA, 20 bp, Artificial sequence
  Extension primer
caggttgggaacagcccaaa

SEQ ID NO: 568 - DNA, 19 bp, Artificial sequence
  Extension primer
agagtgtggcctgctcaag

SEQ ID NO: 569 - DNA, 20 bp, Artificial sequence
  Extension primer
gactcatcttgcatggcacc

SEQ ID NO: 570 - DNA, 18 bp, Artificial sequence
  Extension primer
tacacttcctgcagcact

SEQ ID NO: 571 - DNA, 18 bp, Artificial sequence
  Extension primer
tacacttcctgcagcact

SEQ ID NO: 572 - DNA, 21 bp, Artificial sequence
  Extension primer
cttcaaatccttagcccatgc

SEQ ID NO: 573 - DNA, 19 bp, Artificial sequence
  Extension primer
ctcgagcctacctcagcag

SEQ ID NO: 574 - DNA, 24 bp, Artificial sequence
  Extension primer
gcaatttcagtgtattcataggcc

SEQ ID NO: 575 - DNA, 24 bp, Artificial sequence
  Extension primer
ggagggccggcccgagagcctcag

SEQ ID NO: 576 - DNA, 21 bp, Artificial sequence
  Extension primer
ggtaaagcagaggaacctaat

SEQ ID NO: 577 - DNA, 20 bp, Artificial sequence
  Extension primer
tggcagcaggtgtttgccat

SEQ ID NO: 578 - DNA, 18 bp, Artificial sequence
  Extension primer
cgggtgtctggggaggga

SEQ ID NO: 579 - DNA, 23 bp, Artificial sequence
  Extension primer
ggtggttgacatcaggaaggcat

SEQ ID NO: 580 - DNA, 24 bp, Artificial sequence
  Extension primer
tacaggagaagctgttatcacccc

SEQ ID NO: 581 - DNA, 19 bp, Artificial sequence
  Extension primer
caggcctccctcttcttgt

SEQ ID NO: 582 - DNA, 20 bp, Artificial sequence
  Extension primer
gcataagagggatccagcag

SEQ ID NO: 583 - DNA, 19 bp, Artificial sequence
  Extension primer
tgcacaggctgctctgcta

SEQ ID NO: 584 - DNA, 17 bp, Artificial sequence
  Extension primer
ctccccacctcggccag

SEQ ID NO: 585 - DNA, 17 bp, Artificial sequence
  Extension primer
ctccccacctcggccag

SEQ ID NO: 586 - DNA, 22 bp, Artificial sequence
  Extension primer
gaaacacacaaagcaacagaga

SEQ ID NO: 587 - DNA, 19 bp, Artificial sequence
  Extension primer
ttccttggacgaaagcccc

SEQ ID NO: 588 - DNA, 19 bp, Artificial sequence
  Extension primer
ttccttggacgaaagcccc

SEQ ID NO: 589 - DNA, 24 bp, Artificial sequence
  Extension primer
ttggttctgcagtaagaataaatg

SEQ ID NO: 590 - DNA, 24 bp, Artificial sequence
  Extension primer
ctgcctttcccttgtaccattggc

SEQ ID NO: 591 - DNA, 22 bp, Artificial sequence
  Extension primer
agtaactaagtgtgagagaaac

SEQ ID NO: 592 - DNA, 23 bp, Artificial sequence
  Extension primer
tgcgaaaggcaaatttctcgaaa

SEQ ID NO: 593 - DNA, 24 bp, Artificial sequence
  Extension primer
ttcgcacaggctcagggcagctgg

SEQ ID NO: 594 - DNA, 23 bp, Artificial sequence
  Extension primer
ccctgatctcactaactatgtaa

SEQ ID NO: 595 - DNA, 18 bp, Artificial sequence
  Extension primer
ggagatgagcaccgggct

SEQ ID NO: 596 - DNA, 23 bp, Artificial sequence
  Extension primer
gacctgaccaagattgggaagtc

SEQ ID NO: 597 - DNA, 22 bp, Artificial sequence
  Extension primer
cctcactcaaaggaaatccaaa

SEQ ID NO: 598 - DNA, 22 bp, Artificial sequence
  Extension primer
cctcactcaaaggaaatccaaa

SEQ ID NO: 599 - DNA, 19 bp, Artificial sequence
  Extension primer
ccgggtccaataaccctcc

SEQ ID NO: 600 - DNA, 22 bp, Artificial sequence
  Extension primer
cttccggccgcggctcggttct

SEQ ID NO: 601 - DNA, 19 bp, Artificial sequence
  Extension primer
tgagggctcacatcatgga

SEQ ID NO: 602 - DNA, 18 bp, Artificial sequence
  Extension primer
cctctcgcagcgacggct

SEQ ID NO: 603 - DNA, 17 bp, Artificial sequence
  Extension primer
gccaggcaccccacccc

SEQ ID NO: 604 - DNA, 17 bp, Artificial sequence
  Extension primer
agccttctcctcaaccc

SEQ ID NO: 605 - DNA, 20 bp, Artificial sequence
  Extension primer
actaccaaggtcaagaccca

SEQ ID NO: 606 - DNA, 23 bp, Artificial sequence
  Extension primer
agaatcacttgcacagcttatca

SEQ ID NO: 607 - DNA, 18 bp, Artificial sequence
  Extension primer
gcacctgacctcgctggc

SEQ ID NO: 608 - DNA, 17 bp, Artificial sequence
  Extension primer
gggaccaagagggagga

SEQ ID NO: 609 - DNA, 20 bp, Artificial sequence
  Extension primer
acatatccgcgagggactgg

SEQ ID NO: 610 - DNA, 17 bp, Artificial sequence
  Extension primer
gcctgcagcatgggtcc

SEQ ID NO: 611 - DNA, 17 bp, Artificial sequence
  Extension primer
gcctgcagcatgggtcc

SEQ ID NO: 612 - DNA, 23 bp, Artificial sequence
  Extension primer
ccccagagaccacaaagcaccat

SEQ ID NO: 613 - DNA, 23 bp, Artificial sequence
  Extension primer
tggccaaaccatatttccatcct

SEQ ID NO: 614 - DNA, 23 bp, Artificial sequence
  Extension primer
tggccaaaccatatttccatcct

SEQ ID NO: 615 - DNA, 22 bp, Artificial sequence
  Extension primer
caccttccaaattattgagtgg

SEQ ID NO: 616 - DNA, 17 bp, Artificial sequence
  Extension primer
acctaccacgtctgccc

SEQ ID NO: 617 - DNA, 17 bp, Artificial sequence
  Extension primer
acaacggcaggagggga

SEQ ID NO: 618 - DNA, 17 bp, Artificial sequence
  Extension primer
acaacggcaggagggga

SEQ ID NO: 619 - DNA, 17 bp, Artificial sequence
  Extension primer
atggcccgcctcggccc

SEQ ID NO: 620 - DNA, 20 bp, Artificial sequence
  Extension primer
ttctctgcagttcagtctcc

SEQ ID NO: 621 - DNA, 24 bp, Artificial sequence
  Extension primer
caaaccagacctttttgctgatat

SEQ ID NO: 622 - DNA, 19 bp, Artificial sequence
  Extension primer
gaggaggagatgctaaccc

SEQ ID NO: 623 - DNA, 18 bp, Artificial sequence
  Extension primer
aaaccacgccccagcagc

SEQ ID NO: 624 - DNA, 23 bp, Artificial sequence
  Extension primer
cctcgccagggctgcggctttat

SEQ ID NO: 625 - DNA, 21 bp, Artificial sequence
  Extension primer
agaaccgtatctggcacataa

SEQ ID NO: 626 - DNA, 20 bp, Artificial sequence
  Extension primer
gctaattaggccttaacact

SEQ ID NO: 627 - DNA, 20 bp, Artificial sequence
  Extension primer
ccttatcctcctcctgggaa

SEQ ID NO: 628 - DNA, 23 bp, Artificial sequence
  Extension primer
gctctttcccaagtgccctctgt

SEQ ID NO: 629 - DNA, 20 bp, Artificial sequence
  Extension primer
ccttcctgatcatgctcctc

SEQ ID NO: 630 - DNA, 20 bp, Artificial sequence
  Extension primer
atgacctgggcttgccctca

SEQ ID NO: 631 - DNA, 23 bp, Artificial sequence
  Extension primer
tagaagtcttttgatcccagtcc

SEQ ID NO: 632 - DNA, 22 bp, Artificial sequence
  Extension primer
gagagagaaggaaaagtgatgc

SEQ ID NO: 633 - DNA, 21 bp, Artificial sequence
  Extension primer
tctttaatgactgagccatgc

SEQ ID NO: 634 - DNA, 23 bp, Artificial sequence
  Extension primer
gtatccatgtttttctttcccct

SEQ ID NO: 635 - DNA, 20 bp, Artificial sequence
  Extension primer
cctcaggccaaacaaacaac

SEQ ID NO: 636 - DNA, 23 bp, Artificial sequence
  Extension primer
gagttctgtagtttttgttgcca

SEQ ID NO: 637 - DNA, 24 bp, Artificial sequence
  Extension primer
ccatttaaaatcaaatacaacagc

SEQ ID NO: 638 - DNA, 20 bp, Artificial sequence
  Extension primer
gagcaccttctgagatggca

SEQ ID NO: 639 - DNA, 22 bp, Artificial sequence
  Extension primer
ttaatcaactgactactgctct

SEQ ID NO: 640 - DNA, 19 bp, Artificial sequence
  Extension primer
aattctcagccactgcggc

SEQ ID NO: 641 - DNA, 18 bp, Artificial sequence
  Extension primer
cggagcgtggcctcgatc

SEQ ID NO: 642 - DNA, 19 bp, Artificial sequence
  Extension primer
tgcatacctcggtttcccc

SEQ ID NO: 643 - DNA, 17 bp, Artificial sequence
  Extension primer
ggatttcagcagagtgg

SEQ ID NO: 644 - DNA, 17 bp, Artificial sequence
  Extension primer
tctcattacagccccat

SEQ ID NO: 645 - DNA, 24 bp, Artificial sequence
  Extension primer
tgcaacgagggcggagcttgtgca

SEQ ID NO: 646 - DNA, 24 bp, Artificial sequence
  Extension primer
ggtttgagcaaaggagcttaaaat

SEQ ID NO: 647 - DNA, 20 bp, Artificial sequence
  Extension primer
atggaacccaccaactcaag

SEQ ID NO: 648 - DNA, 22 bp, Artificial sequence
  Extension primer
tagactgtcactttggagatgg

SEQ ID NO: 649 - DNA, 23 bp, Artificial sequence
  Extension primer
taggtatggagttacctttaacg

SEQ ID NO: 650 - DNA, 19 bp, Artificial sequence
  Extension primer
agcagctctgcccagtgaa

SEQ ID NO: 651 - DNA, 24 bp, Artificial sequence
  Extension primer
gtcggggttgatctcgatggtgat

SEQ ID NO: 652 - DNA, 24 bp, Artificial sequence
  Extension primer
gtcggggttgatctcgatggtgat

SEQ ID NO: 653 - DNA, 22 bp, Artificial sequence
  Extension primer
ggcagagacatgaaaagaaaaa

SEQ ID NO: 654 - DNA, 24 bp, Artificial sequence
  Extension primer
atctttggaagttttagaagaaat

SEQ ID NO: 655 - DNA, 17 bp, Artificial sequence
  Extension primer
caggaaagccatgcaac

SEQ ID NO: 656 - DNA, 20 bp, Artificial sequence
  Extension primer
gaaatctttagaaatcccca

SEQ ID NO: 657 - DNA, 24 bp, Artificial sequence
  Extension primer
cagtttgggcaaattagtttgcac

SEQ ID NO: 658 - DNA, 24 bp, Artificial sequence
  Extension primer
cagtttgggcaaattagtttgcac

SEQ ID NO: 659 - DNA, 19 bp, Artificial sequence
  Extension primer
ccccatcagttgcccactc

SEQ ID NO: 660 - DNA, 19 bp, Artificial sequence
  Extension primer
ggcatggatggaagccaag

SEQ ID NO: 661 - DNA, 17 bp, Artificial sequence
  Extension primer
ggcgcccacggctgcac

SEQ ID NO: 662 - DNA, 20 bp, Artificial sequence
  Extension primer
gcgcccggcgcttgacttca

SEQ ID NO: 663 - DNA, 20 bp, Artificial sequence
  Extension primer
tctgtgtctaacctcatgcc

SEQ ID NO: 664 - DNA, 19 bp, Artificial sequence
  Extension primer
ggcccggctcagccccagt

SEQ ID NO: 665 - DNA, 24 bp, Artificial sequence
  Extension primer
gtcaccatataaagatgaacttca

SEQ ID NO: 666 - DNA, 19 bp, Artificial sequence
  Extension primer
gcatgcggagcccgggaac

SEQ ID NO: 667 - DNA, 20 bp, Artificial sequence
  Extension primer
ctggaggggatggagccaag

SEQ ID NO: 668 - DNA, 24 bp, Artificial sequence
  Extension primer
gatgggaactatttggctcatcag

SEQ ID NO: 669 - DNA, 22 bp, Artificial sequence
  Extension primer
acacctaaactagagatgatgt

SEQ ID NO: 670 - DNA, 19 bp, Artificial sequence
  Extension primer
ctcatttgccaccgtaggg

SEQ ID NO: 671 - DNA, 24 bp, Artificial sequence
  Extension primer
tgtcagcatcattactagaggaac

SEQ ID NO: 672 - DNA, 24 bp, Artificial sequence
  Extension primer
cagaaaactcactgaaaggttatt

SEQ ID NO: 673 - DNA, 24 bp, Artificial sequence
  Extension primer
catgagagtatacagataaagata

SEQ ID NO: 674 - DNA, 21 bp, Artificial sequence
  Extension primer
aatgaaggtggaaggtacatc

SEQ ID NO: 675 - DNA, 21 bp, Artificial sequence
  Extension primer
aatgaaggtggaaggtacatc

SEQ ID NO: 676 - DNA, 17 bp, Artificial sequence
  Extension primer
ggagggtgaaagaatgc

SEQ ID NO: 677 - DNA, 17 bp, Artificial sequence
  Extension primer
ggagggtgaaagaatgc

SEQ ID NO: 678 - DNA, 19 bp, Artificial sequence
  Extension primer
acttcacgagcagtcaggc

SEQ ID NO: 679 - DNA, 21 bp, Artificial sequence
  Extension primer
tgtttgaattggtcagggaag

SEQ ID NO: 680 - DNA, 21 bp, Artificial sequence
  Extension primer
ctcctttccttaaaccctcat

SEQ ID NO: 681 - DNA, 24 bp, Artificial sequence
  Extension primer
tcttgaggaaaaaaaagattagtt

SEQ ID NO: 682 - DNA, 21 bp, Artificial sequence
  Extension primer
gaggcaaacagtttgaaaagc

SEQ ID NO: 683 - DNA, 21 bp, Artificial sequence
  Extension primer
gaggcaaacagtttgaaaagc

SEQ ID NO: 684 - DNA, 22 bp, Artificial sequence
  Extension primer
gcaaggagttccacatgtcaag

SEQ ID NO: 685 - DNA, 22 bp, Artificial sequence
  Extension primer
gcaaggagttccacatgtcaag

SEQ ID NO: 686 - DNA, 23 bp, Artificial sequence
  Extension primer
tgcctgaggatcaaaactcagag

SEQ ID NO: 687 - DNA, 24 bp, Artificial sequence
  Extension primer
ggggaaaggaatttgcctgaacaa

SEQ ID NO: 688 - DNA, 19 bp, Artificial sequence
  Extension primer
tgggagcctcaacgtactc

SEQ ID NO: 689 - DNA, 20 bp, Artificial sequence
  Extension primer
ccatacctgttggtggagca

SEQ ID NO: 690 - DNA, 19 bp, Artificial sequence
  Extension primer
tggtctactagggccctga

SEQ ID NO: 691 - DNA, 23 bp, Artificial sequence
  Extension primer
ggacaccaaagaagacgaaaatg

SEQ ID NO: 692 - DNA, 23 bp, Artificial sequence
  Extension primer
gtccaatccagaagccactagct

SEQ ID NO: 693 - DNA, 10 bp, Artificial sequence
  DNA oligomer
acgttggatg